(19) United States
(12) Patent Application Publication
(10) Pub. No.: US 2002/0138872 A1
(43) Pub. Date: Sep. 26, 2002

(54) ACQUIRED RESISTANCE GENES AND USES THEREOF

(76) Inventors: XINNIAN DONG, DURHAM, NC (US); FREDERICK M. AUSUBEL, NEWTON, MA (US); HUI CAO, DURHAM, NC (US); JANE GLAZEBROOK, COLUMBIA, MD (US)

Correspondence Address: CLARK & ELBING, 176 FEDERAL STREET, BOSTON, MA, 02110

(21) Appl. No.: 08/908,884

(22) Filed: Aug. 8, 1997

Related U.S. Application Data
(60) Provisional application No. 60/023,851, filed on Aug. 9, 1996.
(60) Provisional application No. 60/035,166, filed on Jan. 10, 1997.
(60) Provisional application No. 60/046,769, filed on May 16, 1997.

(51) Int. Cl. C12N 15/82

(52) U.S. Cl. 800/279

(57) ABSTRACT

Genomic and cDNA sequences encoding plant acquired resistance proteins are disclosed. Expression of these polypeptides in transgenic plants are useful for providing enhanced defense mechanisms to combat plant diseases

### CROSS REFERENCE TO RELATED APPLICATIONS

This application claims benefit from provisional applications 60/023,851, 60/035,166, and 60/046,769, filed on Aug. 9, 1996, Jan. 10, 1997, and May 16, 1997 respectively.

### STATEMENT AS TO FEDERALLY SPONSORED RESEARCH

This invention was made in part with Government funding, and the Government therefore has certain rights in the invention. In particular, portions of the invention disclosed herein were funded, in part, by USDA Grant Nos. 93-37301-8925, 95-37301-1917, and 94-373033-0464, and NIH RO1 GM48707.

### BACKGROUND OF THE INVENTION

This invention relates to the fields of genetic engineering, plant biology, plant pathogen defense genes and their proteins, and crop protection.

Recent advances in plant pathology have provided a basis for understanding the cellular and molecular genetic mechanisms by which plants defend themselves against pathogen attack. In particular, plants are known to utilize at least two different types of defense mechanisms: (i) the hypersensitive response (“HR”) and (ii) acquired resistance (“AR”), including systemic acquired resistance (“SAR”) and local acquired resistance (“LAR”). These defense mechanisms are discussed below.

The Hypersensitive Response

Plants respond in a variety of ways to pathogenic microorganisms (Lamb, Cell 76:419-422, 1994; Lamb et al., Cell 56:215-224, 1989). One well-studied defense response that occurs at the site of infection is called the hypersensitive response (“HR”) and involves rapid localized necrosis of the infected plant cells or tissue or both. The rapid death of the infected cells is thought to deprive invading pathogens of a sufficient nutrient supply, arresting pathogen growth. Cells undergoing a HR exhibit nuclear DNA fragmentation (for example, DNA laddering), a hallmark of apoptosis first described in animal systems, indicating that the HR involves active, programmed cell death (Mittler et al., Plant Physiol. 108:489-493, 1995; Greenberg et al., Cell 77: 551-563, 1994; Ryerson and Heath, Plant Cell 8:393-402, 1996; Wang et al., Plant Cell 8, 375-391, 1996). The HR is also accompanied by a membrane-associated oxidative burst that results in the NADPH-dependent production of O₂ and H₂O₂. These reactive oxygen species may be directly toxic to invading pathogens or may be involved in the crosslinking of plant cell walls surrounding the lesion to form a barrier to infection (Bradley et al., Cell 70:21-30, 1992; Levine et al., Cell 79:583-593, 1994).

In the 1950s, H. H. Flor developed a well-known genetic model that explains the observation that some races (strains) of a particular pathogen elicited a strong HR on a given cultivar of a host species, whereas other races (strains) of the same pathogen proliferated and caused disease (Flor, Annu. Rev. Phytopathol. 9:275-296, 1971). A pathogen that elicits an HR is said to be avirulent on that host, the host is said to be resistant, and the plant-pathogen interaction is said to be incompatible. In contrast, strains which cause disease on a particular host are said to be virulent, the host is said to be susceptible, and the plant-pathogen interaction is said to be compatible. In many cases, the molecular basis of incompatibility appears to be due to a gene-for-gene correspondence between pathogen “avirulence” (avr) genes and host “resistance” (R) genes (Flor, Annu. Rev. Phytopathol. 9:275-296, 1971). A plant carrying a particular resistance gene will be resistant to pathogens carrying the corresponding avr gene. A simple molecular explanation for this gene-for-gene correspondence between avr and R genes is that avr genes generate signals for which resistance genes encode the cognate receptors. A signal transduction pathway then carries the avr-generated signal to a set of target genes which initiates the HR and other host defenses (Gabriel and Rolfe, Annu. Rev. Phytopathol. 28:365-391, 1990; Keen, Plant Mol. Biol. 19:109-122, 1992; Lamb et al., Cell 56:215-224, 1989).

A variety of avr genes have been cloned from bacterial and fungal phytopathogens (Keen, Plant Mol. Biol. 19:109-122, 1992) and, in at least two cases, gene-for-gene interactions have been demonstrated by experiments showing that a purified avr-generated signal molecule will elicit an HR (Culver and Dawson, Mol. Plant-Microbe Interact. 4:458-463, 1991; Joosten et al., Nature 367:384-386, 1994; Knorr and Dawson, Proc. Natl. Acad. Sci., USA 85:170-174, 1988; van den Ackerveken et al., Plant J. 7:359-366, 1992). Several plant resistance genes have also been cloned in the past four years that conform to a classic gene-for-gene relationship. These include the tomato PTO gene (resistance to strains of P. syringae pv tomato expressing the avirulence gene avrPto (Martin et al., Science 262:1432-1436, 1993)), the Arabidopsis RPS2 and RPM1 genes (resistance to P. syringae expressing the avirulence genes avrRpt2 or avrRpm1, respectively (Bent et al., Science 265:1856-1860, 1994; Grant et al., Science 269:843-846 1995; Mindrinos et al., Cell 78:1089-1099, 1994)), the tobacco N gene (resistance to tobacco mosaic virus (Whitham et al., Cell 78:1101-1105, 1994)), the tomato Cƒ9 and Cƒ2 genes (resistance to the fungal pathogen C. fulvum (Dixon et al., Cell 84:451-459, 1996; Jones et al., Science 266, 789-794, 1994)), the flax L₆ gene (resistance to the fungal pathogen Melampsora lini (Lawrence et al., Plant Cell 7:1195-1206, 1995)), and the rice Xa21 gene (resistance to Xanthomonas oryzae (Song et al., Science 270:1804-1806, 1995)).

Acquired Resistance—Systemic and Local Acquired Resistance

The HR not only blocks the local growth of an infecting pathogen, it is also thought to trigger additional defense responses in uninfected parts of the plant which become resistant to a variety of normally virulent pathogens (Enyedi et al., Cell 70:879-886, 1992; Malamy and Klessig, Plant J. 2:643-654, 1992). This latter phenomenon is called systemic acquired resistance (SAR) and is thought to be the consequence of the concerted activation of many genes that are often referred to as pathogenesis-related (“PR”) genes. The biological functions of many of these PR genes remain unknown; however, a large body of physiological, biochemical, and molecular evidence suggests that particular PR genes play a direct role in conferring resistance to pathogens. For example, some PR genes encode chitinases and β-1,3-glucanases which directly inhibit pathogen growth in vitro (Mauch et al., Plant Physiol. 88:936-942, 1988; Ponstein et al., Plant Physiol. 104:109-118, 1994; Schlumbaum et al., Nature 324:365-367, 1986; Sela-Buurlage et al., Plant Physiol. 101:857-863, 1993; Terras et al., J. Biol. Chem. 267:15301-15309, 1992; Woloshuk et al., Plant Cell 3:619-628, 1991). In addition, constitutive expression in transgenic plants of PR genes has been shown to decrease disease susceptibility in a limited number of cases (Alexander et al., Proc Natl. Acad. Sci. USA 90:7327-7331, 1993; Liu et al., Proc. Natl. Acad. Sci. USA 91:1888-1892, 1994; Terras et al., Plant Cell 7:573-588, 1995; Zhu et al., Bio/Technology 12:807-812, 1994).

SAR was originally defined by Ross (Virology 14:340-358, 1961), who demonstrated that tobacco became resistant to infection by a number of viruses after a primary inoculation with an avirulent strain of tobacco mosaic virus. Subsequently, it was demonstrated that SAR could also be elicited by other viruses, bacteria, and fungi, and that the resistance induced by any particular pathogen was effective against a broad spectrum of viral, bacterial, and fungal diseases (Cameron et al., Plant J. 5:715-725, 1994; Cruikshank and Mandryk, J. Aust. Inst. Agric. Sci. 26:369-372, 1960; Dempsey et al., Phytopathology 83:1021-1029, 1993; Hecht and Bateman, Phytopathology 54:523-530, 1964; Kuc, BioScience 39:854-860, 1982; Lovrekovich et al., Phytopathology 58:1034-1035, 1968; Mauch-Mani and Slusarenko, Mol. Plant-Microbe Interact. 7:378-383, 1994; Uknes et al., Mol. Plant-Microbe Interact. 6:692-698, 1993).

Another acquired plant defense response that shares many features with SAR is so-called local acquired resistance or “LAR.” LAR develops in the direct vicinity of a successfully proliferating pathogen to block further spread of the pathogen and to thwart the occurrence of secondary infections. The same set of PR proteins is believed to be involved in conferring resistance by both LAR and SAR, and, as described below, the same signalling molecules also appear to be required for the onset of both responses.

Certain chemicals, such as salicylic acid (SA), 2,6-dichloroisonicotinic acid (INA), and benzo(1,2,3)thiadiazole-7-carbothioic acid S-methyl ester (BTH) have been shown to induce SAR or LAR or both when applied exogenously to plants (White, Virology 99:410-412, 1979; Metraux et al., Science 250:1004-1006, 1991; Görlach et al., Plant Cell 8:629-643, 1996). Moreover, several lines of evidence indicate that endogenously produced SA is involved in the signal transduction pathway(s) coupling HR with the onset of SAR. In tobacco and cucumber, an increase in SA concentration has been observed after an avirulent pathogen infection when accompanied by the establishment of SAR (Goodman and Plurad, Physiol. Plant. Pathol. 1:11-16, 1971; Malamy et al., Science 250:1002-1004, 1990; Metraux et al., Science 250:1004-1006, 1990; Rasmussen et al., Plant Physiol. 97:1342-1347, 1991). The accumulation of SA is also associated with the subsequent induction of genes including those encoding PR proteins (Van Loon and Van Kammen, Virology 40:199-211, 1970; Ward et al., Plant Cell 3:1085-1094, 1991; Yalpani et al., Plant Cell 3:809-818, 1991). In tobacco and Arabidopsis, exogenously applied SA can induce the accumulation of PR mRNAs, which is a characteristic of SAR (Uknes et al., Plant Cell 4:645-656, 1992; Ward et al., Plant Cell 3:1085-1094, 1991; White, Virology 99:410-412, 1979).

These results have led to the hypothesis that one of the consequences of pathogen infection is the accumulation of SA in vivo, which induces the expression of a set of proteins that act to limit further infection of the host (Ward et al., Plant Cell 3:1085-1094, 1991). Direct support for this hypothesis has come from the observation that transgenic tobacco or Arabidopsis plants that express a bacterial gene encoding a salicylate hydroxylase are unable to accumulate SA and, consequently, do not exhibit either SAR or LAR (Gaffney et al., Science 261:754-756, 1993). Thus, SA is thought to be required in vivo for the establishment of SAR and LAR, and, as described above, PR gene products appear to participate directly in conferring pathogen resistance.

### SUMMARY OF THE INVENTION

In general, the invention features an isolated nucleic acid molecule including a sequence encoding an acquired resistance (AR) polypeptide, wherein the acquired resistance polypeptide is at least 40% (and preferably 50%, 70%, 80%, or 90%) identical to the amino acid sequence of FIG. 5 (SEQ ID NO:3) or FIG. 7B (SEQ ID NO: 14). Preferably, such a nucleic acid molecule encodes an acquired resistance polypeptide that mediates the expression of a pathogenesis-related polypeptide. In another preferred embodiment, the acquired resistance polypeptide includes an ankyrin-repeat motif.

Nucleic acid molecules of the invention are derived from any plant species, including, without limitation, angiosperms (for example, dicots and monocots) and gymnosperms. Exemplary plants from which the nucleic acid may be derived include, without limitation, sugar cane, wheat, rice, maize, sugar beet, potato, barley, manioc, sweet potato, soybean, sorghum, cassava, banana, grape, oats, tomato, millet, coconut, orange, rye, cabbage, apple, watermelon, canola, cotton, carrot, garlic, onion, pepper, strawberry, yam, peanut, onion, bean, pea, mango, and sunflower. Preferred nucleic acid molecules are derived from cruciferous plants, for example, Arabidopsis thaliana. Examples of cruciferous acquired resistance molecules are shown in FIG. 4 (NPR genomic DNA; SEQ ID NO:1) and FIG. 5 (NPR cDNA; SEQ ID NO:2). Other preferred nucleic acid molecules are derived from solanaceous plants, for example, Nicotiana glutinosa. An example of such a solanaceous acquired resistance molecule is shown in FIG. 7A (SEQ. ID NO:13).

In another aspect, the invention features an isolated nucleic acid molecule (for example, a DNA molecule) that encodes an acquired resistance polypeptide that specifically hybridizes to a nucleic acid molecule that includes the nucleic acid sequence of FIG. 4 (NPR genomic DNA; SEQ ID NO:1), FIG. 5 (NPR cDNA; SEQ ID NO:2), or FIG. 7A (SEQ ID NO:13). Preferably, the specifically hybridizing nucleic acid molecule encodes an acquired resistance polypeptide that mediates the expression of a pathogenesis-related polypeptide. In another preferred embodiment, the specifically hybridizing nucleic acid molecule encodes an acquired resistance polypeptide including an ankyrin-repeat motif. In yet other preferred embodiments, the specifically hybridizing nucleic acid molecule complements an acquired resistance mutant (for example, an Arabidopsis npr mutant). The invention also features an RNA transcript having a sequence complementary to any of the isolated nucleic acid molecules described above.

In related aspects, the invention further features a cell or a vector (for example, a plant expression vector), each of which includes an isolated nucleic acid molecule of the invention. In preferred embodiments, the cell is a bacterium (for example, E. coli or Agrobacterium tumefaciens) or is a plant cell (for example, is a cell from any of the crops listed above). Such a plant cell has an increased level of resistance against a disease caused by a plant pathogen (for example, Phytophthora, Peronospora, or Pseudomonas). In yet another preferred embodiment, the isolated nucleic acid molecule of the invention is operably linked to an expression control region that mediates expression of a polypeptide encoded by the nucleic acid molecule. For example, the expression control region is capable of mediating constitutive, inducible (for example, pathogen- or wound-inducible), or cell- or tissue-specific gene expression. The invention further features a cell (for example, a bacterium such as E. coli or Agrobacterium tumefaciens, or a plant cell) which contains the vector of the invention.

In still another aspect, the invention features a transgenic plant including any of the above nucleic acid molecules of the invention integrated into the genome of the plant, wherein the nucleic acid molecule is expressed in the transgenic plant. In addition, the invention features seeds and cells from such transgenic plants. For example, such transgenic plants may be produced according to conventional methods using any of the above crop plants.

In yet another aspect, the invention features a substantially pure acquired resistance polypeptide including an amino acid sequence that has at least 40% (and preferably, 50%, 60%, 70%, 80% or 90%) identity to the amino acid sequence of FIG. 5 (SEQ ID NO:3) or FIG. 7B (SEQ ID NO:14). Preferably, the acquired resistance polypeptide mediates the expression of a pathogenesis-related polypeptide. In other preferred embodiments, the acquired resistance polypeptide includes an ankyrin-repeat motif or a G-protein coupled receptor motif. Such acquired resistance polypeptides are derived from any plant species, for example, those crop plants mentioned above. In preferred embodiments, the polypeptide of the invention is derived from a cruciferous species, for example, Arabidopsis thaliana, or from a solanaceous species, for example, Nicotiana glutinosa.

In a related aspect, the invention also features a method of producing an acquired resistance polypeptide. The method involves: (a) providing a cell transformed with a nucleic acid molecule of the invention positioned for expression in the cell; (b) culturing the transformed cell under conditions for expressing the nucleic acid molecule; and (c) recovering the acquired resistance polypeptide. The invention further features a recombinant acquired resistance polypeptide produced by such expression of an isolated nucleic acid molecule of the invention, and a substantially pure antibody that specifically recognizes and binds to an acquired resistance polypeptide or a portion thereof.

In another aspect, the invention features a method of providing an increased level of resistance against a disease caused by a plant pathogen in a transgenic plant. The method involves: (a) producing a transgenic plant cell including the nucleic acid molecule of the invention integrated into the genome of the transgenic plant cell and positioned for expression in the plant cell; and (b) growing a transgenic plant from the plant cell wherein the nucleic acid molecule is expressed in the transgenic plant and the transgenic plant is thereby provided with an increased level of resistance against a disease caused by a plant pathogen.

In another aspect, the invention features methods of isolating an acquired resistance gene or fragment thereof. The first method involves: (a) contacting the nucleic acid molecule of the invention or a portion thereof with a preparation of DNA from a plant cell under hybridization conditions providing detection of DNA sequences having 40% or greater sequence identity to the nucleic acid sequence of FIG. 4 (SEQ ID NO:1), FIG. 5 (SEQ ID NO:2), or FIG. 7A (SEQ ID NO:13); and (b) isolating the hybridizing DNA as an acquired resistance gene or fragment thereof. The second method involves: (a) providing a sample of plant cell DNA; (b) providing a pair of oligonucleotides having sequence homology to a region of a nucleic acid molecule of the invention; (c) contacting the pair of oligonucleotides with the plant cell DNA under conditions suitable for polymerase chain reaction-mediated DNA amplification; and (d) isolating the amplified acquired resistance gene or fragment thereof.

In preferred embodiments of the second method, the amplification step is carried out using a sample of cDNA prepared from a plant cell. In addition, the pair of oligonucleotides used in the second method are based on a sequence encoding an acquired resistance polypeptide, wherein the acquired resistance polypeptide is at least 40% (and preferably 50%, 60%, 70%, 80%, or 90%) identical to the amino acid sequence of FIG. 5 (SEQ ID NO:3) or FIG. 7B (SEQ ID NO:14).

By “acquired resistance” gene or “AR” gene is meant a gene encoding a polypeptide capable of triggering a plant acquired resistance response (for example, a systemic acquired resistance (SAR) or local acquired resistance response (LAR)) in a plant cell or plant tissue. This response may occur at the transcriptional level or it may be enzymatic or structural in nature. AR genes may be identified and isolated from any plant species, especially agronomically important crop plants, using any of the sequences disclosed herein in combination with conventional methods known in the art.

By “polypeptide” is meant any chain of amino acids, regardless of length or post-translational modification (for example, glycosylation or phosphorylation).

By “pathogenesis-related” polypeptide or “PR” polypeptide is meant a polypeptide that is expressed in conjunction with the establishment of SAR or LAR. Exemplary PR proteins include, without limitation, chitinase, PR-1a, PR1, PR5, GST (glutathione-S-transferase), and β-1,3 glucanase, osmotin, thionin, glycine-rich proteins (GRPs), phenylalanine ammonia lyase (PAL), and lipoxygenase (LOX).

By “ankyrin-repeat” motif is meant a consensus motif that is found in a wide variety of proteins that are capable of mediating protein-protein interactions. Ankyrin-repeat motifs are described in Michaely and Bennett (Trends in Cell Biology 2:127-129, 1992) and Bork (Proteins: Structure, Function, and Genetics 17:363-374, 1993).

By “substantially identical” is meant a polypeptide or nucleic acid exhibiting at least 40%, preferably 50%, more preferably 80%, and most preferably 90%, or even 95% homology to a reference amino acid sequence (for example, the amino acid sequence shown in FIG. 5 (SEQ ID NO:3) or FIG. 7B (SEQ ID NO:14)) or nucleic acid sequence (for example, the nucleic acid sequences shown in FIG. 4, or FIG. 5, or FIG. 7A, SEQ ID NOS:1, 2, and 13, respectively). For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, or PILEUP/PRETTYBOX programs). Such software matches identical or similar sequences by assigning degrees of homology to various substitutions, deletions, and/or other modifications. Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine.

By a “substantially pure polypeptide” is meant an AR polypeptide (for example, an NPR polypeptide such as NPR1) that has been separated from components which naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, an AR polypeptide. A substantially pure AR polypeptide may be obtained, for example, by extraction from a natural source (for example, a plant cell); by expression of a recombinant nucleic acid encoding an AR polypeptide; or by chemically synthesizing the protein. Purity can be measured by any appropriate method, for example, column chromatography, polyacrylamide gel electrophoresis, or by HPLC analysis.

By “derived from” is meant isolated from or having the sequence of a naturally-occurring sequence (e.g., a cDNA, genomic DNA, synthetic, or combination thereof).

By “isolated DNA” is meant DNA that is free of the genes which, in the naturally-occurring genome of the organism from which the DNA of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. It also includes a recombinant DNA which is part of a hybrid gene encoding additional polypeptide sequence.

By “specifically hybridizes” is meant that a nucleic acid sequence is capable of hybridizing to a DNA sequence at least under low stringency conditions as described herein, and preferably under high stringency conditions, also as described herein.

By “transformed cell” is meant a cell into which (or into an ancestor of which) has been introduced, by means of recombinant DNA techniques, a DNA molecule encoding (as used herein) an AR polypeptide.

By “positioned for expression” is meant that the DNA molecule is positioned adjacent to a DNA sequence which directs transcription and translation of the sequence (i.e., facilitates the production of, for example, an AR polypeptide, a recombinant protein, or an RNA molecule).

By “reporter gene” is meant a gene whose expression may be assayed; such genes include, without limitation, β-glucuronidase (GUS), luciferase, chloramphenicol transacetylase (CAT), green fluorescent protein (GFP), β-galactosidase, herbicide resistant genes and antibiotic resistance genes.

By “expression control region” is meant any minimal sequence sufficient to direct transcription. Included in the invention are promoter elements that are sufficient to render promoter-dependent gene expression controllable for cell-, tissue-, or organ-specific gene expression, or elements that are inducible by external signals or agents (for example, light-, pathogen-, wound-, stress-, or hormone-inducible elements or chemical inducers such as SA or INA); such elements may be located in the 5′ or 3′ regions of the native gene or engineered into a transgene construct.

By “operably linked” is meant that a gene and a regulatory sequence(s) are connected in such a way as to permit gene expression when the appropriate molecules (for example, transcriptional activator proteins) are bound to the regulatory sequence(s).

By “plant cell” is meant any self-propagating cell bounded by a semi-permeable membrane and containing a plastid. Such a cell also requires a cell wall if further propagation is desired. Plant cell, as used herein includes, without limitation, algae, cyanobacteria, seeds, suspension cultures, embryos, meristematic regions, callus tissue, leaves, roots, shoots, gametophytes, sporophytes, pollen, and microspores.

By “crucifer” is meant any plant that is classified within the Cruciferae family. The Cruciferae include many agricultural crops, including, without limitation, rape (for example, Brassica campestris and Brassica napus), broccoli, cabbage, brussel sprouts, radish, kale, Chinese kale, kohlrabi, cauliflower, turnip, rutabaga, mustard, horseradish, and Arabidopsis.

By “transgene” is meant any piece of DNA which is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism.

By “transgenic” is meant any cell which includes a DNA sequence which is inserted by artifice into a cell and becomes part of the genome of the organism which develops from that cell. As used herein, the transgenic organisms are generally transgenic plants and the DNA (transgene) is inserted by artifice into the nuclear or plastidic genome. A transgenic plant according to the invention may contain one or more acquired resistance genes.

By “pathogen” is meant an organism whose infection of viable plant tissue elicits a disease response in the plant tissue. Such pathogens include, without limitation, bacteria, mycoplasmas, fungi, insects, nematodes, viruses, and viroids. Plant diseases caused by these pathogens are described in Chapters 11-16 of Agrios, Plant Pathology, 3rd ed., Academic Press, Inc., New York, 1988.

Examples of bacterial pathogens include, without limitation, Erwinia (for example, E. carotovora), Pseudomonas (for example, P. syringae), and Xanthomonas (for example, X. campepestris and X. oryzae).

Examples of fungal disease-causing pathogens include, without limitation, Alternaria (for example, A. brassicola and A.solani), Ascochyta (for example, A. pisi), Botrytis (for example, B. cinerea), Cercospora (for example, C. kikuchii and C. zaeamaydis), Colletotrichum sp. (for example, C. lindemuthianum), Diplodia (for example, D. maydis), Erysiphe (for example, E. graminis f.sp. graminis and E. graminis f.sp. hordei), Fusarium (for example, F. nivale and F. oxysporum, F. graminearum, F. solani, F. monilforme, and F. roseum), Gaeumanomyces (for example, G. graminis f.sp. tritici), Helminthosporium (for example, H. turcicum, H. carbonum, and H. maydis), Macrophomina (for example, M. phaseolina and Maganaporthe grisea), Nectria (for example, N. heamatocacca), Peronospora (for example, P. manshurica, P. tabacina), Phoma (for example, P. betae), Phymatotrichum (for example, P. omnivorum), Phytophthora (for example, P. cinnamomi, P. cactorum, P. phaseoli, P. parasitica, P. citrophthora, P. megasperma f.sp. sojae, and P. infestans), Plasmopara (for example, P. viticola), Podosphaera (for example, P. leucotricha), Puccinia (for example, P. sorghi, P. striiformis, P. graminis f.sp. tritici, P. asparagi, P. recondita, and P. arachidis), Puthium (for example, P. aphanidermatum), Pyrenophora (for example, P. tritici-repentens), Pyricularia (for example, P. oryzea), Pythium (for example, P. ultimum), Rhizoctonia (for example, R. solani and R. cerealis), Scerotium (for example, S. rolfsii), Sclerotinia (for example, S. sclerotiorum), Septoria (for example, S. lycopersici, S. glycines, S. nodorum and S. tritici), Thielaviopsis (for example, T. basicola), Uncinula (for example, U. necator), Venturia (for example, V. inaequalis), Verticillium (for example, V. dahliae and V. albo-atrum).

Examples of pathogenic nematodes include, without limitation, root-knot nematodes (for example, Meloidogyne sp. such as M. incognita, M. arenaria, M. chitwoodi, M. hapla, M. javanica, M. graminocola, M. microtyla, M. graminis, and M. naasi), cyst nematodes (for example, Heterodera sp. such as H. schachtii, H. glycines, H. sacchari, H. oryzae, H. avenae, H. cajani, H. elachista, H. goettingiana, H. graminis, H. mediterranea, H. mothi, H. sorghi, and H. zeae, or, for example, Globodera sp. such as G. rostochiensis and G. pallida), root-attacking nematodes (for example, Rotylenchulus reniformis, Tylenchuylus semipenetrans, Pratylenchus brachyurus, Radopholus citrophilus, Radopholus similis, Xiphinema americanum, Xiphinema rivesi, Paratrichodorus minor, Heterorhabditis heliothidis, and Bursaphelenchus xylophilus), and above-ground hematodes (for example, Anguina funesta, Anguina tritici, Ditylenchus dipsaci, Ditylenchus myceliphagus, and Aphenlenchoides besseyi).

Examples of viral pathogens include, without limitation, tobacco mosaic virus, tobacco necrosis virus, potato leaf roll virus, potato virus X, potato virus Y, tomato spotted wilt virus, and tomato ring spot virus.

By “increased level of resistance” is meant a greater level of resistance to a disease-causing pathogen in a transgenic plant (or cell or seed thereof) of the invention than the level of resistance relative to a control plant (for example, a non-transgenic plant). In preferred embodiments, the level of resistance in a transgenic plant of the invention is at least 20% (and preferably 30% or 40%) greater than the resistance of a control plant. In other preferred embodiments, the level of resistance to a disease-causing pathogen is 50% greater, 60% greater, and more preferably even 75% or 90% greater than a control plant; with up to 100% above the level of resistance as compared to a control plant being most preferred. The level of resistance is measured using conventional methods. For example, the level of resistance to a pathogen may be determined by comparing physical features and characteristics (for example, plant height and weight, or by comparing disease symptoms, for example, delayed lesion development, reduced lesion size, leaf wilting and curling, water-soaked spots, and discoloration of cells) of transgenic plants.

By “detectably-labelled” is meant any direct or indirect means for marking and identifying the presence of a molecule, for example, an oligonucleotide probe or primer, a gene or fragment thereof, or a cDNA molecule or a fragment thereof. Methods for detectably-labelling a molecule are well known in the art and include, without limitation, radioactive labelling (for example, with an isotope such as ³²P or ³⁵S) and nonradioactive labelling (for example, chemiluminescent labelling, for example, fluorescein labelling).

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody, for example, an acquired resistance polypeptide-specific antibody. A purified AR antibody may be obtained, for example, by affinity chromatography using a recombinantly-produced acquired resistance polypeptide and standard techniques.

By “specifically binds” is meant an antibody which recognizes and binds an AR protein but which does not substantially recognize and bind other molecules in a sample, for example, a biological sample, which naturally includes an AR protein such as NPR.

As discussed above, fundamental acquired resistance genes that are responsible for providing plants with the ability to protect themselves against pathogens have been identified. Accordingly, the invention provides a number of important advances and advantages for the protection of plants against their pathogens. For example, by providing AR genes as described herein that are readily incorporated and expressed in all species of plants, the invention facilitates an effective and economical means for in-plant protection against plant pathogens. Such protection against pathogens reduces or minimizes the need for traditional chemical practices (for example, application of fungicides, bactericides, nematicides, insecticides, or viricides) that are typically used by farmers for controlling the spread of plant pathogens and providing protection against disease-causing pathogens. In addition, because plants expressing one or more acquired resistance gene(s) described herein are less vulnerable to pathogens and their diseases, the invention further provides for increased production efficiency, as well as for improvements in quality and yield of crop plants and ornamentals. Thus, the invention contributes to the production of high quality and high yield agricultural products: for example, fruits, ornamentals, vegetables, cereals and field crops having reduced spots, blemishes, and blotches that are caused by pathogens; agricultural products with increased shelf-life and reduced handling costs; and high quality and yield crops for agricultural (for example, cereal and field crops), industrial (for example, oilseeds), and commercial (for example, fiber crops) purposes. Furthermore, because the invention reduces the necessity for chemical protection against plant pathogens, the invention benefits the environment where the crops are grown. Genetically-improved seeds and other plant products that are produced using plants expressing the genes described herein also render farming possible in areas previously unsuitable for agricultural production. The invention further provides a means for mediating the expression of pathogenesis-related proteins, for example, chitinase and GST, that confer resistance to plant pathogens. For example, transgenic plants constitutively producing an AR gene product are capable of activating PR gene expression, which in turn confers resistance to plant pathogens. Collective PR gene expression that is mediated by the AR gene product obviates the need to express individual PR genes as a means to promote plant defense mechanisms.

The invention is also useful for providing nucleic acid and amino acid sequences of an AR gene that facilitates the isolation and identification of AR genes from any plant species.

Other features and advantages of the invention will be apparent from the following description of the preferred embodiments thereof, and from the claims.

### DETAILED DESCRIPTION

The drawings will first be described.

### DRAWINGS

FIG. 1 is a schematic illustration showing the physical map of A. thaliana chromosome I and the position of NPR1.

FIG. 2A is a photograph of a Northern blot analysis showing the expression of the PR-1 gene in wild type plants(Co1-0, lanes 1-3), npr1-2 mutant plants(lanes 4-6), npr1-2 transformants with a noncomplementing cosmid (m305-2-7, lanes 7-9), and npr1-2 transformants with complementing cosmids (21A4-P5-1, lanes 10-12 and 21A4-6-1-1, lanes 13-15). RNA samples were prepared from fifteen-day old seedlings grown on MS media (lanes 1, 4, 7, 10, and 13), MS media with 0.1 mM INA (lanes 2, 5, 8, 11, and 14), and MS media with 0.1 mM SA (lanes 3, 6, 9, 12, and 15).

FIG. 2B is a series of photographs showing disease symptoms (top panels) and BGL2-GUS expression (bottom panels) induced by Psm ES4326 on wild-type (left panels), npr1-1 (middle panels), and an npr1-1 transformant with a complementing cosmid (21A4-4-3-1, right panels).

FIG. 2C is a panel of graphs showing the growth of Psm ES4326 in wild-type, npr1-2, and an npr1-2 transformant with a complementing cosmid (21A4-P5-1). Error bars represent 95% confidence limits of log-transformed data as described by Sokal and Rohlf (Biometry, 2d ed., W. H. Freeman and Company, New York, 1981).

FIG. 2D is a panel of bar graphs showing the disease rating of P. parasitica NOCO infection in wild type, npr1-2, and an npr1-2 transformant with a complementing cosmid (21A4-P5-1). The disease rating scales are defined as follows: 0, no conidiophores on the plant; 1, no more than 5 conidiophores per infected leaf; 2, 3-20 conidiophores on a few infected leaves; 3, 6-20 condiophores on most infected leaves; 4, 5 or more conidiophores on all infected leaves; 5, 20 or more conidiophores on all infected leaves.

FIG. 3 is a schematic illustration showing the restriction map of the 7.5-kb region containing the NPR1 gene.

FIG. 4 is a schematic illustration showing the genomic sequence of the 7.5-kb region containing the acquired resistance nucleic acid sequence of the gene termed NPR1 (SEQ ID NO:1) from Arabidopsis thaliana.

FIG. 5 is a schematic illustration showing the cDNA sequence (SEQ ID NO:2) and deduced amino acid sequence (SEQ ID NO:3) of the acquired resistance protein termed NPR1 from Arabidopsis thaliana. Amino acids numbered 262-289, 323-371, and 453-469 show homology to a mouse ankyrin protein, an ankyrin-repeat motif, and a G-protein coupled receptor motif, respectively.

FIG. 6A is a schematic illustration showing the alignment of the NPR1 amino acid sequence with mouse ankyrin 3 (ANKB). Two regions producing the highest scoring pairs (smallest sum probability=0.0004) generated using a BLAST search are shown. The identical and similar amino acids (+) are highlighted in bold, circled letters.

FIG. 6B is a schematic illustration showing the alignment of the ankyrin repeats in NPR1 with the ankyrin repeat consensus derived from Michaely and Bennett (Trends in Cell Biology 2:127-129, 1992) and Bork (Proteins: Structure, Function, and Genetics 17:363-374, 1993). Since there are a few non-overlapping amino acids between the two derived consensus sequences, both are presented. In the consensus derived from Bork, the conserved features are indicated: t, turn-like or polar; o, S/T; h, hydrophobic; capitals, conserved amino acids. Those amino acids identical to the consensus are highlighted in bold, circled letters.

FIG. 7A is a schematic illustration showing the cDNA sequence (SEQ ID NO:13) of an NPR1 homolog isolated from Nicotiana glutinosa.

FIG. 7B is a schematic illustration showing the deduced amino acid sequence of the NPR1 homolog of Nicotiana glutinosa (SEQ ID NO:14) shown in FIG. 7A.

FIG. 8A is a graph illustrating the dosage effect of NPR1 on the resistance of transgenic Arabidopsis to the bacterial pathogen, Psm ES4326. Eight samples were taken at each time point for the Psm ES4326 infection (initial inoculant OD₆₀₀=0.001). Error bars represent 95% confidence limits of log-transformed data. Colony forming unit is designated as cfu.

FIG. 8B is a histogram showing the dosage effect of NPR1 on the resistance of transgenic Arabidopsis to the fungal pathogen, Peronspora parasitica NOCO2. A spore suspension (3×10⁴ spores/mL) of P. parasitica was used for these infection studies, and the number of conidiophores on each plant was counted seven days after infection. The data were analyzed using Wilcoxon two-sample tests. At the 95% confidence level, significant difference in growth was present between all pairs of samples except Co1NPR1-M and Co1NPR1-H, and Co1 and Co1NPR1-L.

FIG. 9A are photographs showing the restoration of inducible BGL2-GUS expression in 35S-NPR1-GFP transgenic plants. Seedlings were grown on either MS or MS-INA (0.1 mM) media for fourteen days and stained for GUS activity.

FIG. 9B is a photograph showing the complementation of the SA sensitivity in the Arabidopsis npr1 mutant by 35S-NPR1-GFP. Seedlings were grown for eleven days on MS-SA (0.5 mM) medium. The NPR1-GFP transgene restored normal growth to npr1 on SA. The mGFP transgene, however, was unable to restore normal growth to npr1. Note that the NPR1-GFP line used was in the T₂ generation. The observed 3:1 segregation ratio indicated that the transgenic plants contained a single locus NPR1-GFP insertion.

FIG. 9C is a histogram showing the restoration of P. parasitica resistance to the T₂ NPR1-GFP transformants. INA treatment (0.65 mM) was carried out seventy-two hours prior to infection with a spore suspension (3×10⁴ spores/mL). The disease symptoms were scored seven days after the infection with respect to the number of conidiophores on the plant. The disease rating scale is defined as: 0, no conidiophores on the plant; 1, no more than 5 conidiophores per infected leaf, 2, 6-20 conidiophores on a few infected leaves; 3, 6-20 conidiophores on most of the infected leaves; 4, 5 or more conidiophores on all infected leaves; 5, 20 or more conidiophores on all infected leaves. Seedlings in the 0, 4, and 5 categories were also examined for the presence of the NPR1-GFP transgene, and the number of NPR1-GFP transformants is indicated in the parenthesis. Most of the P. parasitica resistant plants (0 category) contained the NPR1-GFP transgene; however, all of the sensitive plants (4 and 5 categories) were observed to segregate as non-transformants lacking the transgene.

FIG. 10 is a photograph showing the localization of NPR1-GFP in response to chemical activators of SAR. The transformants, containing either the NPR1-GFP (top and bottom panels) or mGFP transgene (middle panels) were grown for eleven days on MS or MS-INA media. GFP fluorescence was visualized by confocal microscopy in leaf mesophyll cells and guard cells. DIC is shown in the red channel and GFP is shown in the green channel.

FIGS. 11A-11G are a series of photographs showing the localization of NPR1-GFP in response to Psm ES4326 infection. Leaves of NPR1-GFP transformants were infiltrated on the left half with either Psm ES4326 (FIG. 11B) or 10 mM MgCl₂ (FIG. 11E) and stained for BGL2-GUS expression after three days. Prior to GUS staining the leaves were analyzed for GFP localization on the infiltrated (FIG. 11A and FIG. 11D) and the uninfiltrated (FIG. 11C) side. Leaves of mGFP transformants were infiltrated with Psm ES4326 (FIG. 11F) or 10 mM MgCl₂ (FIG. 11G) and analyzed for GFP localization.

### OVERVIEW

A genetic study was conducted using Arabidopsis thaliana as a model system to identify key elements that control the signaling pathway leading to the induction of acquired resistance (AR), for example, a system acquired resistance (SAR) response, to pathogen infection in plants. In wild-type Arabidopsis plants, SAR responses can be induced by treatment with 0.1 mM salicylic acid (SA) or 0.1 mM 2,6-dichloroisonicotinic acid (INA) or after an infection by an avirulent pathogen such as Pseudomonas syringae pv phaseolicola NP3121/avrRpt2 (P.s. phaseolicola 3121/avrRpt2). SAR is demonstrated by enhanced resistance to virulent pathogens, such as Pseudomonas syringae pv maculicola ES4326 (P.s. maculicola ES4326), and by increased expression of pathogenesis-related genes (for example, PR genes including PR1, BGL2, and PR5). To facilitate detection of PR gene expression and identification of mutants that were aberrant in the SAR signaling pathway, a BGL2-GUS reporter gene was constructed and transformed into Arabidopsis thaliana ecotype Columbia. This parental line containing the BGL2-GUS transgene was mutagenized by treatment of seeds with 0.3% ethyl methanesulfonate for eleven hours. The M2 progeny of the mutagenized population were screened for the lack of BGL2-GUS expression in the presence of the SAR-inducers SA and INA (Cao et al., Plant Cell 6:1583-1592, 1994).

Using these techniques, the npr1-1 (nonexpresser of PR genes) mutant was isolated and found to have almost complete lack of expression of the BGL2-GUS reporter gene, as well as a lack of expression of the endogenous PR1, BGL2, and PR5 genes in response to SA, INA, and avirulent pathogen treatments (Cao et al., Plant Cell 6:1583-1592, 1994). Further characterization of the npr1-1 mutant showed that mutations in the NPR1 gene completely blocked the induction of SAR. In the npr1-1 plants pretreated with SA, INA, or an avirulent pathogen, growth of virulent pathogens (for example, P.s. maculicola ES4326) was not inhibited, as found in the parental line carrying the wild-type NPR1 gene. This finding demonstrated that the NPR1 gene plays a key role in the signaling pathway leading to the establishment of SAR.

Two additional npr1 mutants, npr1-2 and npr1-3, were isolated on the basis that they were more susceptible to infection than wild-type plants by P.s. maculicola strain ES4326 (Glazebrook et al., Genetics 143:973-982, 1996). Genetic complementation tests showed that npr1-1, npr1-2, and npr1-3 were allelic.

The NPR1 gene not only controls the onset of systemic resistance, but also was found to affect local acquired resistance (“LAR”), the ability of plants to restrict the spread of virulent pathogen infections. In npr1 mutant plants, the virulent pathogen P.s. maculicola ES4326 grows to a greater extent and spreads further beyond the initial site of invasion than in the wild-type plants. The effects of the impaired SAR and LAR in npr1 mutants is also evident when various strains of Peronospora parasitica were tested. Disease symptoms (i.e., downy mildew) were observed after infection by strains of P. parasitica to which the wild-type parental line of Arabidopsis is resistant, showing the break down of the “natural” resistance in the npr1 mutants. The effects of the npr1 mutations appeared to be specific to the defense response. No significant morphological phenotypes were observed in three allelic npr1 mutants, npr1-1, npr1-2, npr1-3. However, when grown on medium containing a high concentration of SA (0.5 mM), the growth of all three npr1 mutants was arrested at the cotyledon stage, and the seedlings were bleached. Wild-type plants were observed to grow normally in the presence of 0.5 mM SA.

The phenotypes of the npr1 mutants clearly demonstrated the biological significance of the NPR1 gene of Arabidopsis thaliana in controlling the defense response against a broad spectrum of pathogens.

The NPR1 gene was cloned using a map-based positional cloning strategy. The location of NPR1 on the Arabidopsis genome was first delimited to a 7.5-kilobase (kb) region contained on cosmid clones 21A4-4-3-1, 21A4-6-1-1, 21A4-P5-1, 21A4-P4-1, and 21A4-2-1 by its ability to complement the npr1 mutant. An SA-inducible 2.0-kb RNA transcript encoded within this 7.5-kb region corresponding to NPR1 was identified by RNA blot analysis. Isolation of this acquired resistance gene facilitates the cloning of AR genes from plants of agricultural or economic importance. For example, engineering ectopic expression of AR genes (for example, an NPR gene) in crop plants, which is useful for providing novel strategies for creating plants with enhanced resistance to pathogen infection.

There now follows a description of the cloning of an Arabidopsis AR gene, NPR1. A description is also provided of the cloning of the NPR1 homolog from Nicotiana glutinosa. These examples are provided for the purpose of illustrating the invention, and should not be construed as limiting.

Genetic Analysis of SAR in Arabidopsis and the Isolation of npr1 Mutants

Using Arabidopsis thaliana, components of the signalling pathway in SAR downstream of SA and INA induction have been identified. Specifically, we sought Arabidopsis mutants that did not express PR genes in the presence of added SA or INA. Because there is no visible phenotype known to be associated with such mutants, transgenic Arabidopsis plants were generated which expressed β-glucuronidase (GUS) under the control of the Arabidopsis β-1,3-glucanase (BGL2) promoter (Dong et al., Plant Cell 3:61-72, 1991). The BGL2 gene is one of the PR genes regulated by SA (Uknes et al., Plant Cell 4:645-656, 1992). Briefly, seed from the transgenic line (BGL2-GUS) were mutagenized with ethyl methanesulfonate (EMS), and the resulting mutants were screened after SA or INA treatment for aberrant expression of GUS. The results of these screenings showed that high levels of β-glucuronidase (GUS) activity could be assayed in a single well of a ninety-six well microtiter plate using a single leaf from a plant that had been grown for two weeks on plates containing SA or INA. Screens were performed for Arabidopsis mutants that either expressed the BGL2-GUS reporter constitutively in the absence of SA or INA treatment or that failed to express the reporter gene following treatment with SA or INA. These screens led to the identification of a series of mutants called cpr and npr (constitutive expresser of PR genes and for non-expresser of PR genes, respectively) which define genes that are involved both in the regulation of BGL2 specifically and SAR in general (Bowling et al., Plant Cell 6:1845-1857, 1994; Cao et al., Plant Cell 6:1583-1592, 1994).

Construction of BGL2-GUS Transgenic Arabidopsis

An XbaI-SphI fragment (2025 base pairs (bp)) containing 1746-bp of noncoding sequence upstream of the start codon of the Arabidopsis BGL2 gene was fused at the ATG site to the coding region of the Escherichia coli uidA gene (referred to as the GUS gene) and transferred into the vector pBI101, which was then used to transform Arabidopsis ecotype Columbia (Valvekens et al., Proc. Natl. Acad. Sci. USA 85:5536-5540, 1988). Plants homozygous for the BGL2-GUS construct were identified on the basis that progeny of these plants were resistant to kanamycin and the presence of the transgene that was detected using Southern hybridization.

Mutagenesis of the BGL2-GUS Transgenic Line

Mutagenesis was performed in the BGL2-GUS/BGL2-GUS transgenic line by exposing ˜36,000 seeds to 0.3% ethyl methanesulfonate for eleven hours. Seeds were sown, and the plants were allowed to self-fertilize to produce M₂ seeds, which were collected in twelve independent pools.

Identification of the npr1-1 Mutant

The M₂ seeds were germinated on MS medium with the addition of 0.8% agar, 0.5 mg/mL Mes (2-(N-morpholino)ethane-sulfonic acid), pH 5.7, 2% sucrose, 50 μg/mL kanamycin, and 100 μg/mL ampicillin. Either 0.5 mM salicylic acid (SA) or 0.1 mM INA was added to induce systemic acquired resistance (SAR). After incubation for fifteen days, each seedling to be assayed was numbered, and a single leaf was then removed from each seedling and put into the corresponding sample well of a ninety-six-well microtiter plate that contained 100 μL of β-glucuronidase (GUS) substrate solution (50 mM Na₂HPO₄, pH 7.0, 10 mM Na₂EDTA, 0.1% Triton X-100, 0.1% sarkosyl, 0.7 μL/mL βmercaptoethanol, and 0.7 mg/mL 4-methylumbelliferyl β-D-glucuronide). After all the samples were collected, the microtiter plate was placed under vacuum for two minutes to infiltrate the samples and then incubated at 37° C. overnight. Samples were examined for the fluorescent product of GUS activity (4-methylumbellifone) using a long-wavelength UV light. Those seedlings which showed no GUS activity were identified on the MS plate and transplanted to soil for seed setting. This procedure was repeated in the progeny of these putative mutants to ensure that the mutant phenotype was heritable and to identify the homozygous mutants. Of 13,468 M₂ plants tested, 181 did not exhibit GUS activity in the presence of either SA or INA. In the M₃ generation, 77 of 139 lines tested maintained a mutant phenotype for GUS activity, with 76 nonresponsive to both SA and INA and one line nonresponsive to SA but responsive to INA.

Three classes of mutations were predicted to be carried by the mutants that were nonresponsive to SA or INA treatment: (1) mutations in regulatory genes which not only affect expression of the transgene, but also the endogenous PR genes; (2) mutations in the promoter of the transgene which affect the responsiveness of BGL2-GUS, but not that of the endogenous PR genes to SA and INA; and (3) mutations in the coding region of the GUS gene which abolish the enzymatic activity of GUS, but not the transcription of GUS mRNA. To distinguish between these classes, the expression of endogenous PR genes was analyzed in the M₃ generation. Regulatory gene mutants should be readily distinguished in the M₃ generation by an aberrant level of expression of other SAR-related PR genes.

RNA gel blot analysis was performed with these 77 mutant lines to identify those with modified expression of PR genes. The expression of the Arabidopsis mitochondrial β-ATPase gene served as a control for sample loading. Among the 77 mutant lines, six were found to have reduced expression of the endogenous PR genes to some degree (class 1); three showed aberrant expression only in BGL2-GUS (class 2); and fourteen were found to have reduced GUS activity but normal transcription of BGL2-GUS (class 3). One class 1 mutant (npr1-1) exhibited a dramatic reduction in expression of the GUS, BGL2, and PR-1 genes compared to the wild-type in the presence of SA or INA. Therefore, npr1-1 was selected for further study.

The npr1-1 mutant was tested for the induction of PR-5, another PR gene that has been cloned in Arabidopsis (Uknes et al., Plant Cell 4:645-656, 1992), and a similar reduction in expression was observed. The reduction in PR gene expression after SA or INA treatment was quantified for npr1-1 relative to the parent BGL2-GUS line (representing the wild-type). In npr1-1, the expression of both GUS and BGL2 was ten-fold lower than that of the wild-type and that of PR-5 was five-fold lower. The most dramatic reduction was observed for PR-1 which was twenty-fold lower than the wild-type.

Quantitative GUS Assays Using npr1-1

To measure accurately the level of GUS activity, a quantitative GUS assay was performed on npr1-1 plants and the wild-type BGL2-GUS plants grown in the presence of either SA or INA, or in the absence of both. In the absence of an inducer, the background level of GUS activity was five-fold lower in the npr1-1 mutant than in the wild-type. Wild-type plants grown in the presence of 0.5 mM SA showed a fifty-two-fold increase in GUS activity compared to the uninduced plants, whereas in the SA-induced npr1-1 plants, the increase in GUS activity was only seven-fold. Moreover, the induction by 0.1 mM INA was forty-eight-fold for the wild-type versus five-fold for npr1-1. Thus, while GUS activity in the SA- or INA-treated npr1-1 plants was somewhat induced, the activity was at most only slightly higher than the background level of the untreated wild-type.

Genetic Analysis of the npr1-1 Locus

A backcross of npr1-1/npr1-1 with its wild-type parent (NPR1/NPR1 in the BGL2-GUS background) resulted in F₁ progeny (NPR1/npr1-1, sixteen plants were tested) with the same pattern of GUS staining (using 5-bromo-4-chloro-3-indolyl glucuronide [XGluc] as the substrate) observed in the wild-type after SA or INA treatment. GUS staining was not detected in the SA- or INA-treated npr1-1/npr1-1 homozygous plants even after two days of incubation at 28° C. Self-fertilization of the F₁ plants produced F₂ progeny that segregated for GUS activity, intense staining or complete absence of staining, which were present with a ratio of 219:64 among the 283 F₂ plants examined, demonstrating that the mutant phenotype is recessive and due to a single nuclear mutation (χ²=0.86; P>0.1).

SA-, INA-, and Avirulent Pathogen-Induced Protection Against Pseudomonas syringae pv maculicola ES4326 Infection in Wild-Type and npr1-1

To examine whether the lack of SA- or INA-induced PR gene expression would affect SAR protection against a virulent pathogen infection, fifteen-day-old wild-type and npr1-1 plants were treated with either 1 mM SA or 0.65 mM INA, and two days later were exposed to a P.s. maculicola ES4326 bacterial suspension. Significant protection was observed in the SA- or INA-treated wild-type plants with less than ten percent of plants showing slight yellowing. Chlorotic lesions developed in about ninety percent of the untreated wild-type control plants not pretreated with SA or INA. However, such SA- or INA-induced protection was not observed in npr1-1 mutant plants. Chlorotic lesions were clearly seen in over ninety-percent of untreated and at least eighty-percent of SA- or INA-treated plants. The symptoms on npr1-1 were also more severe than on the wild-type plants. Treatment with only 1 mM SA, 0.65 mM INA, or surfactant (0.01% Silwet-77, used for the bacterial infection) had a minimal effect on both the wild-type and the npr1-1 plants.

The growth of P.s. maculicola ES4326 was measured in both wild-type and npr1-1 plants that had been treated with water, SA, or INA two days before P. s. maculicola ES4326 infection. Leaves were collected 0, 0.5, 1.0, 2.0, and 3.0 days after bacterial infiltration. For the untreated wildtype plants, P.s. maculicola ES4326 proliferated 10,000-fold during this time period. However, for SA- or INA-treated wild-type plants, the growth of P.s. maculicola ES4326 was only about ten-fold, 1000 times lower than the untreated control. A Student's t test of the difference between the means at the three-day time point clearly showed that growth of the pathogen is inhibited in the wild-type plants treated with SA or INA compared to those sprayed with water (P<0.001). Such a dramatic difference in P.s. maculicola ES4326 growth, which resulted from SAR protection, was not observed in the npr1-1 plants, where a Student's t test showed no statistically difference in growth after three days for all conditions (P>0.05); the growth of P.s. maculicola ES4326 in npr1-1 plants was similar for mock-treated and either SA- or INA-treated plants. Comparing the untreated npr1-1 plants with the untreated wild-type, the level of P.s. maculicola ES4326 appeared to have reached saturation one day earlier in the mutant than in the wild-type. Moreover, the difference in P.s. maculicola ES4326 growth between the SA- or INA-treated wild-type and npr1-1 was 500- to 1000-fold.

To test the response to an avirulent pathogen, the npr1-1 plants were infiltrated with P.s. maculicola ES4326 carrying an avirulence gene avrRpt2 as described by Dong et al. (Plant Cell 3:61-72, 1991) and Whalen et al. (Plant Cell 3:49-59, 1991). A typical HR was observed in these npr1-1 plants as characterized by the rapid appearance of necrotic lesions, detection of autofluorescence in the cell wall regions of the infected cells, and inhibited growth of P.s. maculicola ES4326/avrRpt2. The ability of this avirulence gene to induce SAR in npr1-1 plants was then tested. To distinguish the inducing bacterial strain from the challenging strain, the bean pathogen Pseudomonas syringae pv phaseolicola strain NPS3121 (P.s. phaseolicola NPS3121; (Lindgren et al., J. Bacteriol. 168:512-522, 1986)) containing the avrRpt2 gene was used to induce SAR in both the npr1-1 and wild-type plants. P.s. phaseolicola NPS3121 by itself caused no disease symptoms or visible HR on Arabidopsis ecotype Columbia, while P.s. phaseolicola NPS3121/avrRpt2 elicited a strong HR (Yu et al., Mol. Plant-Microbe Interact. 6:434-443, 1993). Three days after the inoculation, uninfected leaves on the same plants were challenged with the virulent pathogen P.s. maculicola ES4326, and the growth of P.s. maculicola ES4326 in the plants was measured. A significant reduction in bacterial growth was observed in the wild-type plants pre-inoculated with P.s. phaseolicola NPS3121/avrRpt2 compared to the mock treated samples (300-fold); however, no difference in P.s. maculicola ES4326 growth was detected in npr1-1 plants.

Disease Symptoms and BGL2-GUS Expression Induced by P.s. maculicola ES4326 Infection in Wild-Type and npr1-1

P.s. maculicola ES4326 was able to establish infection in SA-, INA-, and avirulent pathogen-treated npr1-1 plants as well as in the untreated plants. The lesions formed on the untreated mutant plants and the untreated wild-type were further compared. For this purpose, the P.s. maculicola ES4326 suspension was infiltrated into four-week-old wild-type and npr1-1 leaves. The injection was controlled so that only half of the leaf was infiltrated with the bacteria. This could be monitored by the soaking appearance of the half-leaf. Forty-eight hours following infiltration, chlorotic lesions were visible on the wild-type leaves. These lesions were normally confined to the infiltrated halves of the leaves as defined by the midrib vein. Different lesions were observed on the npr1-1 leaves, where the lesions were more diffuse and often spread into the uninfected halves of the leaves. Sampling of twelve leaves from both wild-type and npr1-1 plants revealed significant growth of the bacteria in the uninoculated half of eleven npr1-1 leaves compared to none of the wild-type leaves.

For the leaves infected with P.s. maculicola ES4326, the pattern of BGL2-GUS expression was examined by X-Gluc staining. In a wild-type leaf, a high level of GUS staining was detected in the peripheral region of the lesion. In contrast, no significant GUS activity was detected on the npr1-1 leaf, where the lesion was more extensive than on the wild-type.

Conclusions About npr1-1

The data described above indicates that npr1-1 harbors a trans-acting mutation(s) affecting the response to SA and INA. The possibility of npr1-1 being a mutant affecting the uptake of exogenously applied SA or INA is ruled out by the observation that the expression of PR1 induced by P.s. maculicola ES4326, instead of by exogenously applied SA or INA, is also reduced in the npr1-1 mutant. The failure of SA or INA to protect the npr1-1 mutant from infection by P.s. maculicola strain ES4326 (in contrast to the protection observed in wild-type plants) indicated that the npr1-1 mutation blocks SA or INA induction of resistance. Even though the HR elicited in the npr1-1 mutant by bacteria carrying the avirulence gene avrRpt2 was similar to that described previously in wild-type plants (Dong et al., Plant Cell 3:61-72, 1991; Whalen et al., Plant Cell 3:49-59, 1991), the HR-induced SAR protection against infection by the virulent pathogen P.s. maculicola ES4326 was absent in the npr1-1 plants. This indicated that npr1-1 is a mutation that prevents the onset of SAR. These phenotypes of the npr1-1 mutation indicated that the function of the wild-type NPR1 gene is to qualitatively and quantitatively regulate the expression of SA- and INA-responsive PR genes.

Genetic analysis of the progeny of an npr1-1/npr1-1 X NPR1/NPR1 backcross indicated that a single recessive nuclear mutation determines the “nonexpresser of PR genes” phenotype of the npr1-1 mutant. This also indicated that the NPR1 gene acts as a positive regulator of SAR responsive gene induction. While the gene could be a negative regulator which is inactivated by SAR induction, a mutation abolishing such regulation would likely be dominant. Furthermore, the fact that a single mutation (that is, npr1-1) affects the responsiveness of this mutant to SA-, INA-, and pathogen induction indicated that SA, INA, and pathogens activate a common pathway that leads to the expression of PR genes.

Identification of the Arabidopsis npr1-2 and npr1-3 Mutants

To identify novel Arabidopsis mutants that negatively affect the induction of SAR, an alternative mutant screening strategy was employed.

We have observed that the final density to which the virulent pathogen P.s. maculicola ES4326 will grow in an Arabidopsis leaf is directly related to the dose at which P.s. maculicola ES4326 was infiltrated. The observed phenotypes of two additional types of Arabidopsis mutants also supported this conclusion. Specifically, a series of Arabidopsis mutants were identified that accumulated reduced levels of the phytoalexin called camalexin, a phytoalexin that has been found in significant quantities in Arabidopsis (Glazebrook and Ausubel, Proc. Natl. Acad. Sci. USA 91:8955-8959, 1994; Tsuji et al., Plant Physiol. 98:1304-1309, 1992). Importantly, P.s. maculicola ES4326 formed disease lesions and grew to higher titers on some of these pad (phytoalexin deficient) mutants when inoculated at doses below the threshold dose required to give disease symptoms in wild-type plants. Similarly, npr1-1 mutants exhibited a similar enhanced susceptibility phenotype as pad mutants (Cao et al., Plant Cell 6:1583-1592, 1994).

Based on these findings that pad and npr mutants were more susceptible to low dose P.s. maculicola ES4326 infection than wild-type plants, a screen was performed to isolate additional eds (enhanced disease susceptibility) mutants (Glazebrook et al., Genetics 143:973-982, 1996). Two leaves of M2 generation mutagenized Arabidopsis plants were infected at a dose of strain P.s. maculicola ES4326 at which wild-type plants showed very weak symptoms manifested as small chlorotic spots three days after infection, whereas pad and npr1 mutants showed large areas of chlorosis. A total of fifteen eds mutants that reproducibly allowed at least one half log more growth of P.s. maculicola ES4326 as compared to wild-type were identified among 12,500 plants screened. Because some pad mutants as well as npr1-1 mutants have the same enhanced susceptibility phenotype with respect to P.s. maculicola ES4326 as the eds mutants (Glazebrook et al., Genetics 143:973-982, 1996), the fifteen eds mutants were tested to determine whether they synthesized wild-type levels of camalexin in response to infection by P.s. maculicola ES4326 (pad phenotype) and whether PR1 gene expression can be induced by salicylic acid (npr1-1 phenotype). The results of these analyses showed that two of the eds mutants exhibited an npr1-like phenotype. Genetic complementation analysis showed that these two mutations are allelic to npr1-1. These two mutants were re-named npr1-2 and npr1-3.

Map-Based Positional Cloning of the Arabidopsis NPR1 Gene

To map the NPR1 gene, a genetic cross was made between the npr1-1 mutant (present in the Columbia ecotype (Col-O) which carried the BGL2-GUS reporter gene) and the wild-type (present in Landsberg erecta ecotype (La-er) which carried the BGL2-GUS reporter gene). F3 families from this cross that are homozygous for this mutation at the NPR1 locus were identified by their lack of expression of BGL2-GUS when grown on plates containing 0.1 mM INA. Expression of the GUS reporter gene was detected by a chromographic assay of GUS activity using the substrate 5-bromo—4-chloro-3-indolyl glucuronide according to standard techniques (Cao et al., Plant Cell 6:1583-1592, 1994 and Jefferson Plant Mol. Biol. Reporter 5:387-405, 1987). The leaf tissues of these F3 npr1-1 progeny pools (from thirty to forty two-week-old seedlings) were collected and frozen in liquid nitrogen. From the frozen tissues, genomic DNA preparations were made as described by Dellaporta et al. (Plant Mol. Biol. Reporter 1:19-21, 1983) and used to determine the genotypes of various restriction fragment length polymorphism (RFLP) and codominant amplified polymorphic sequence (CAPS) (Konieczny and Ausubel, Plant J. 4:403-410, 1993) markers. The frequencies of recombination between the NPR1 locus and the RFLP and CAPS markers were used to determine the position of the NPR1 gene according to conventional methods.

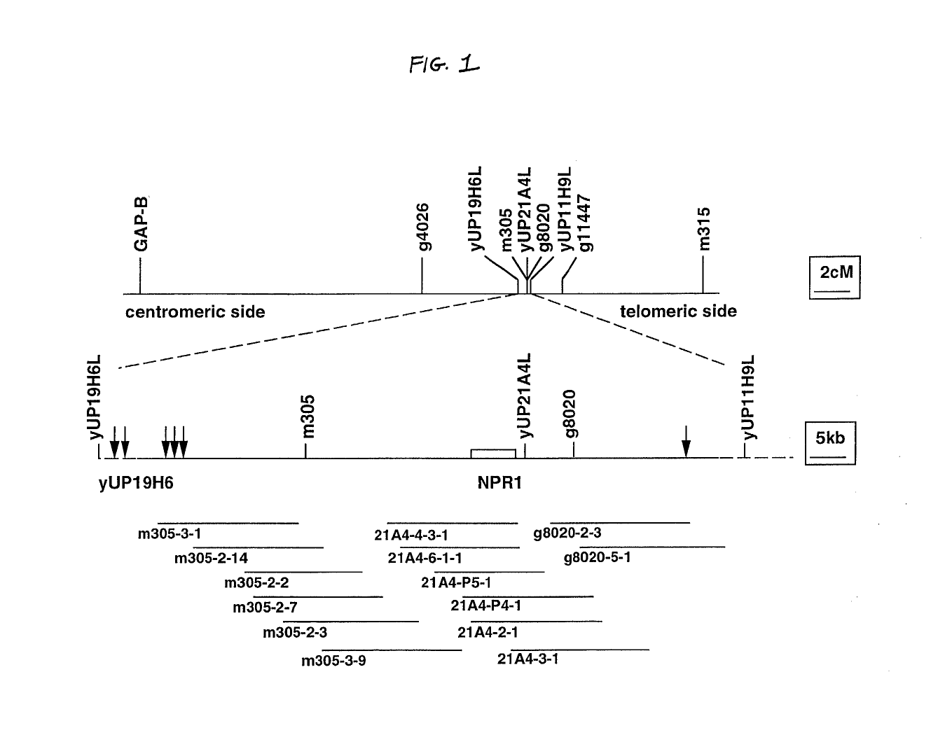

As shown in FIG. 1, the NPR1 gene was mapped to Arabidopsis chromosome I, and found to reside between the CAPS marker GAP-B (˜22.70 cM on the centromeric side of the NPR1 gene) and the RFLP marker m315 (˜7.58 cM on the telomeric side of the NPR1 gene).

To carry out fine mapping of the NPR1 gene, new CAPS and RFLP markers were generated from clones that the genetic maps in the AtDB database (http://genome-www.stanford.edu/Arabidopsis/) showed were located between GAP-B and m315. Cosmid g4026 (CD2-28, Arabidopsis Biological Resource Center, The Ohio State University, Columbus, Ohio) was cut with the restriction enzyme EcoRI and a 4-kb fragment was used to identify a polymorphism between Col-0 and La-er after the genomic DNA was digested with HindIII. Using this RFLP marker, six heterozygotes were detected among the twenty-three F3 families that were heterozygous at GAP-B. None were found among the seven F3 families that were heterozygous at m315. Therefore, g4026 is ˜5.92 cm on the centromeric side of the NPR1 gene. Cosmid g11447 (obtained from the collection of Dr. Howard Goodman at the Massachusetts General Hospital (Nam et al., Plant Cell 1:699-705, 1989)) was used to generate a CAPS marker. End-sequences of an 0.8-kb EcoRI fragment were used to design PCR primers (primer 1:5′ GTGACAGACTTGCTCCTACTG 3′ (SEQ ID NO:15); primer 2:5′ CAGTGTGTATCAAAGCACCA 3′ (SEQ ID NO:16) which amplified a fragment displaying a polymorphism when digested with the EcoRV restriction enzyme. Among the 436 npr1-1 F3 progeny tested using this newly generated CAPS marker, seventeen heterozygotes were discovered. Since these heterozygotes were all homozygous Col-0 for the GAP-B locus, the g11447 marker was placed ˜1.95 cM on the telomeric side of the NPR1 gene.

There are a number of RFLP markers mapped between g11447 and g4026. The first marker tested was m305 (designated CD1-11, Arabidopsis Biological Resource Center, the Ohio State University, Columbus, Ohio (Chang et al., Proc. Natl. Acad. Sci., USA 85:6856-6860, 1988)). A 5-kb EcoRI fragment isolated from the m305 lambda clone was further subcloned using SalI/XbaI and the end-sequences of a 1.6-kb fragment were used to design PCR primers (primer 1:5′ TTCTCCAGACCACATGATTAT 3′(SEQ ID NO:17); primer 2:5′ TGAAGCTAATATGCACAGGAG 3′ (SEQ ID NO:18)). The resulting PCR fragment amplified using these primers was digested with HaeIII to detect a polymorphism. Among the 305 npr1-1 progeny examined using this m305 CAPS marker, no heterozygotes were found, indicating that the m305 marker lies extremely close to NPR1.

A partial physical map of chromosome I (http://cbil.humgen.upenn.edu/˜atgc/ATGCUP.html) showed a YAC contig that includes m305. The YACs in this contig, as well as left-end-fragments of YAC clones yUP19H6, yUP21A4, and yUP11H9 were obtained from Dr. Joseph Ecker at the University of Pennsylvania. The yUP19H6L end-probe was found to detect an RsaI polymorphism, and five recombinants were identified among the GAP-B recombinants on the centromeric side of the NPR1 gene (as shown by the vertical arrows in FIG. 1). The yUP11H9L end-probe was found to detect a HindIII polymorphism, and one heterozygote was found among the seventeen recombinants for gll447 on the telomeric side of the NPR1 gene (as shown by a vertical arrow in FIG. 1). Since yUP11H9L hybridized with the yUP19H6 YAC clone, these results showed that the NPR1 gene is located on yUP19H6. In addition to m305, yUP21A4L (detects an EcoRI polymorphism) and g8020 (a 1.3-kb EcoRI fragment that detects a HindIII polymorphism) were found to be very closely linked to the NPR1 gene with no recombinants identified. m305, yUP21A4L, and g8020 all hybridized to the yUP19H6 YAC clone, further supporting the conclusion that yUP19H6 contains the NPR1 gene.

Construction of a Cosmid Library from the YAC Clone yUP19H6

A genomic DNA preparation was made from the yeast strain containing the YAC clone yUP19H6. This DNA was partially digested with the restriction enzyme TaqI, size selected on a 10-40% sucrose gradient, and cloned into the ClaI site of the binary vector, pCLD04541 (obtained from Dr. Jonathan Jones (Bent et al., Science 265:1856-1860, 1994)). The pCLDO4541 vector is a standard transformation vector used for preparing cosmid libraries. This plasmid carries a T-DNA polylinker region, and tetracycline and kanamycin resistance markers.

The cosmid clones were packaged into bacteriophage lambda particles using a commercial packaging extract (Gigapack XL, Stratagene, LaJolla, Calif.) and introduced into E. coli strain DH5α according to the instructions of the supplier. The resulting library was found to contain approximately 40,000 independent clones.

Generation of a Cosmid Contig Containing the NPR1 Gene

The cosmid library generated from the yeast strain containing yUP19H6 was plated (1,500 cfu/plate) on LB medium agar (containing 5 μg/mL of tetracycline to select for the presence of pCLD04541) and incubated at 37° C. overnight. Colonies were lifted onto membranes (GeneScreen, Du Pont, New England Nuclear) and hybridization was carried out according to the protocol described by the manufacturer. The library was probed with 5-kb EcoRI, 6.5-kb EcoRI/XhoI, and a 1.3-kb EcoRI fragments prepared from m305, yUP21A4L, and g8020, respectively. The colonies that hybridized with these probes were identified and purified according to conventional methods. Cosmid DNA preparations were made from these positive clones using the alkaline lysis method described by Sambrook et al. (Molecular Cloning: A Laboratory Manual, Cold Spring Harbor Laboratory Press, New York, 1989), and the inserts were analyzed by HindIII restriction digestion and Southern hybridization using the probes stated above. The cosmids were found to form a single cosmid contig spanning approximately 80-kb of Arabidopsis DNA. Three of the five recombinants for yUP19HL were shown to be heterozygous at an RFLP marker detected by cosmid clone m305-3-1 (a 5-kb HindIII fragment) at the centromeric side of the contig, while the single heterozygote detected by g8020 marker was also detected by the cosmid clone g8020-6-3 (a 1.25-kb HindlII fragment) at the telomeric side of the contig. This showed that the cosmid contig contained the NPR1 gene (FIG. 1). From this contig, fourteen cosmids which each have a minimum of 10-kb overlap with the neighboring clones (FIG. 1) were chosen to transform npr1 mutant plants in complementation experiments.

Complementation of the npr1 Mutations

The cosmid clones contained in the E. coli strain DH5α were transferred into the Agrobacterium tumefaciens strain GV3101 (pMP90) (Koncz and Schell, Mol. Gen. Genet. 204:383-396, 1986) by conjugation using the helper strain MM294A (pRK2013) (Finan et al., J. Bacteriol. 167:66-72, 1986). The resulting A. tumefaciens conjugants were selected using 50 μg/mL kanamycin and 50 μg/mL gentamycin. The A. tumefaciens strains carrying those fourteen cosmid clones were transformed into npr1-1 (Cao et al., Plant Cell 6:1583-1592, 1994) and npr1-2 (Glazebrook et al., Genetics 143:973-982, 1996) using a vacuum infiltration method described by Bechtold et al. (C.R. Acad. Sci. Paris, Life Sciences 316:1194-1199, 1993). The integrity of the cosmid clones in the A. tumefaciens cultures used for transformation were examined by Southern analysis.

Transformants of npr1-2 were grown (22° C. in fourteen hours of light) and selected on MS medium agar (Murashige and Skoog, Physiol. Plant. 15:473-497, 1962) containing 2% sucrose, 50 μg/mL kanamycin, and 100 μg/mL ampicillin. Kanamycin-resistant transformants which developed true leaves and healthy roots were transplanted to soil. After two weeks of growth in soil at 22° C. in fourteen hours of light per day, leaves were collected from three transformants of each cosmid clone and soaked in 0.5 mM INA solution for twenty-four hours at 22° C. in fourteen hours of light per day. Leaf tissues were then collected and frozen in liquid nitrogen. Total RNA was extracted from these leaf tissues, and an RNA blot was prepared as described by Cao et al. (Plant Cell 6:1583-1592, 1994). The blot was probed with a PR1-specific probe (a PCR product obtained by amplifying genomic Arabidopsis DNA with PR1-specific primers (sense primer 5′ GTAGGTGCTCTTGTTCTTCCC3′ (SEQ ID NO:19); anti-sense primer 5′CACATAATTCCCACGAGGATC3′ (SEQ ID NO:20)).

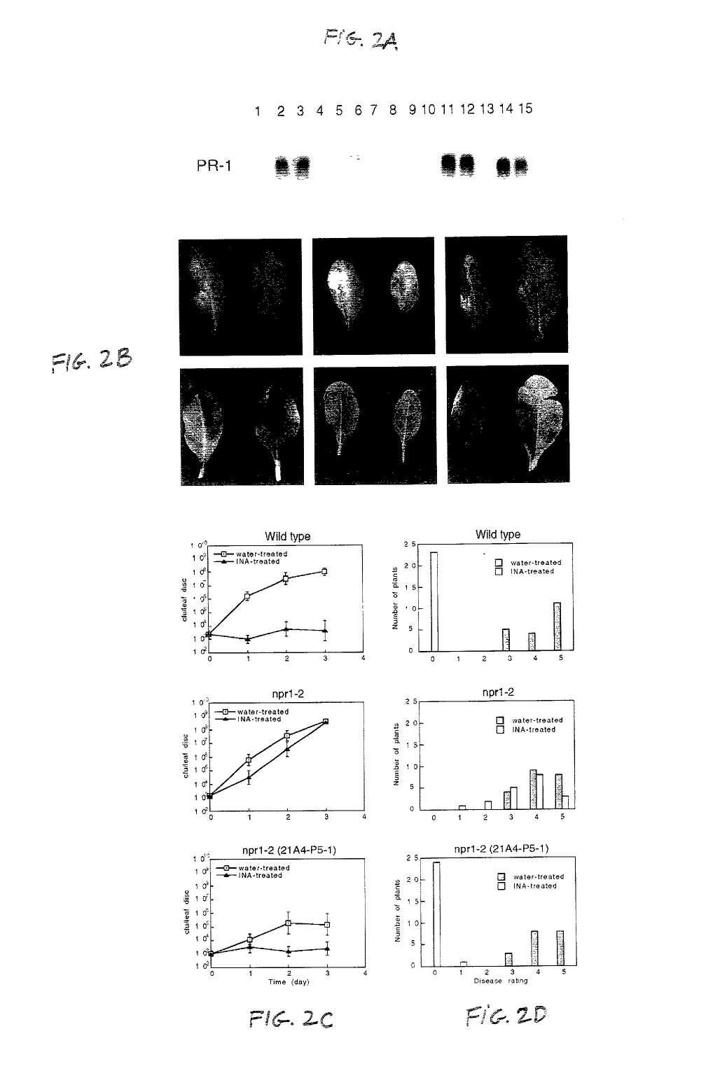

In control experiments, the wild-type parental line showed the induction of the PR1 gene by INA, while the npr1-2 mutant exhibited no induction of PR-1 gene expression. Npr1-2 transformants containing cosmids (three for each cosmid) 21A4-6-1-1, 21A4-P5-1, 21A4-4-3-1, and 21A4-2-1 showed strong induction of PR1 by INA, while npr1-2 transformants containing other clones (for example, M305-2-3, M305-3-9, and 21A4-3-1) displayed no induction. Variations were observed in the intensity of RNA bands among three individual transformants sampled for each cosmid clone. These variations were likely to be the result of “position-effects,” the effect of the insertion site in the chromosome on the expression of the transgene. Cosmid clones 21A4-4-3-1, 21A4-6-1-1, 21A4-P5-1, and 21A4-2-1 restored the ability of the npr1-2 mutant to respond to INA induction and, therefore, complemented the npr1-2 mutation. Examples of INA induced PR1 are shown in FIG. 2A.

Transformants carrying each cosmid were also tested for SA induction of PR1 expression by RNA blot analysis Examples of SA induction are shown in FIG. 2A. The wild-type parental line exhibited a high level of PR1 gene induction by SA, whereas the npr1-2 mutant exhibited only a minor induction (FIG. 2A). Transformants of the npr1-2 mutant containing cosmids 21A4-6-1-1, 21A4-P5-1, 21A4-4-3-1, and 21A4-2-1 showed induction of PR1 by SA, while those containing the other clones displayed little induction.

As shown in FIG. 1, these four clones share a common region of 7.5-kb. Transformants of cosmid 21A4-P4-1 were not available when the experiment described above was conducted. However, according to its relative position, it is expected that this clone can also complement the npr1-2 mutation.

The same fourteen cosmid clones were also transformed into the npr1-1 mutant. Since the npr1-1 mutant carries the BGL2-GUS reporter and the kanamycin resistance gene (NPTII), transformants of the cosmid clones could not be selected using kanamycin. Instead, transformants that complemented the npr1-1 mutation were selected directly by growing the seeds collected from the npr1-1 plants infiltrated with A. tumefaciens on a high concentration of SA (0.5 mM). Those plants that developed green leaves were transplanted to another plate containing 0.1 mM INA, and GUS activity was measured one week after transplanting.

To measure GUS activity, seedlings were numbered, and a single leaf was removed from each plant and placed in a microtiter well containing 100 μL of GUS substrate (4-methylumbelliferyl β-glucuronide) in a solution as described previously (Cao et al., Plant Cell 6:1583-1592, 1994; Jefferson, Plant Mol. Biol. Reporter 5:387-405, 1987). After an overnight incubation at 37° C., the fluorescent product of GUS activity was examined under a long wavelength UV light. As controls, twelve seedlings of the wild-type parental line (BGL2-GUS) were tested, and all showed intense fluorescence after growth on SA and INA. Twelve seedlings of the npr1-1 mutant (BGL2-GUS) were also included in the experiment, and none displayed any increase in fluorescence. From this experiment, nine seedlings carrying cosmid 21A4-P4-1, five carrying 21A4-P5-1, and six carrying 21A4-2-1 were found to have high levels of fluorescence, i.e., GUS activity, and none of the seedlings from other cosmid clones were identified through this selection. Direct identification of putative complementing transformants in the npr1-1 mutant plants by the cosmid clones 21A4-P4-1, 21A4-P5-1, and 21A4-2-1 as in the transformation experiment using the allelic npr1-2 mutant (where all transformants were first selected by kanamycin resistance before identification of the transformants that could complement the npr1-2 mutation using RNA blot analysis) further supported the conclusion from complementation experiments with npr1-2 that the 7.5 kb region shared by cosmids 21A4-4-3-1, 21A4-6-1-1, 21A4-P5-1, 21A4-P4-1, and 21A4-2-1 complemented npr1 mutations, and that this 7.5-kb region contained the NPR1 gene.

In addition to reduced PR gene expression, plants with npr1 mutations display susceptibility to virulent pathogens even after SAR induction. These mutant phenotypes were also complemented by the cosmids described above. For example, as shown in FIG. 2B, infection by the bacterial pathogen Psm ES4326 caused visible disease symptoms three days after infection. While the disease symptoms in the wild-type plants and the complemented npr1-1 transformants were well-confined to the site of pathogen infiltration (the left side of the leaf), the lesions in the npr1-1 plants were found to spread beyond the site of infiltration. In addition, when the dosage of infecting bacteria was reduced 10-fold, severe disease symptoms were only observed in the npr1-1 mutant (leaves on the right). This experiment showed that 21A4-4-3-1 complemented the enhanced susceptibility to Psm ES4326 displayed by npr1-1.

The expression of the BGL2-GUS gene was also analyzed in the same leaves after examination of the disease symptoms (FIG. 2B). Strong GUS expression (blue staining) was detected in the marginal regions of the well-confined lesions in the wild-type plants, but was absent from the diffuse lesions in the npr1-1 plants. Reporter gene expression was restored in complemented transformants.

In addition to these visual observations, as shown in FIG. 2C, bacterial growth of Psm ES4326 was measured quantitatively in wild-type, npr1-2, and an npr1-2 transformant with a complementing cosmid (21A4-P5-1). Plants were treated with 0.65 mM INA seventy-two hours prior to Psm ES4326 infection (0D₆₀₀=0.001). Infection of Arabidopsis with Psm ES4326 was performed according to standard methods (Bowling et al., 1994; supra, Cao et al., supra, 1994; Glazebrook et al., supra, 1996). Samples were taken before infection and one, two, and three days after infection. Six to eight samples were taken for each time point analyzed and colony-forming units of Psm ES4326 were determined per leaf disc. Complete inhibition of Psm ES4326 growth was observed in the wild-type plants following INA treatment three days prior to infection, whereas an approximate 10-fold decrease in Psm ES4326 growth was observed in the npr1-2 mutant subjected to the same treatment. The growth of Psm ES4326 was also halted in the complemented transformants after INA treatment. Lower bacterial growth (as great at 10³-fold) was observed even in the water-treated transformants compared to the water-treated wild-type (FIG. 2C) and the water-treated transformants carrying noncomplementing cosmids. This enhanced resistance may result from the increased NPR1 mRNA levels in these complemented transformants.

A test of resistance to a fungal pathogen, P. parasitica NOCO, was also performed to verify complementation of the npr1-1 mutation. Infection of Arabidopsis with P. parasitica NOCO was performed according to standard methods (Bowling et al., supra, 1994; Cao et al., supra, 1994; Glazebrook et al., supra, 1996). INA treatment (0.65 mM) was carried out seventy-two hours prior to infection with a spore suspension (3×10⁴ spores/1 mL). Seven days post-infection, the disease symptoms were scored with respect to the number of conidiophores observed on each plant. A total of twenty to twenty-five plants were examined for each genotype with each treatment. Data were analyzed using the Mann-Whitney U-Tests (Sokal and Rohlf, supra). As shown in FIG. 2D, the results of these experiments indicated that INA-induced resistance to P. parasitica NOCO was restored in the transformants with the complementing cosmids.

Analyses of the 7.5-kb Region Containing the NPR1 Gene

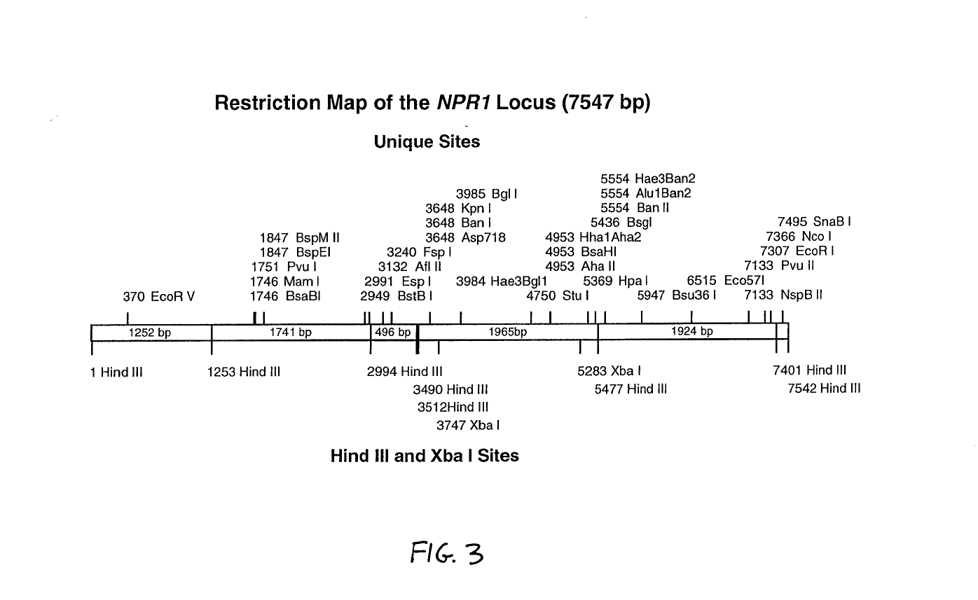

The 7.5-kb region identified by the cosmid complementation experiment was further analyzed using restriction enzymes. The resulting restriction map from this analysis is shown in FIG. 3. Three sets of subclones were made using HindIII, XbaI, and ClaI/XhoI digestions of the cosmid 21A4-P5-1, which has the 7.5-kb region located in the center of the insert, and ligated into the vector pBluescript II SK⁺ (Stratagene, La Jolla, Calif.). The 7.5-kb region of interest was represented by five HindIII subclones with the approximate insert sizes 1.96-kb, 1.91-kb, 1.74-kb, 1.25-kb, and 0.50-kb. Subclones with larger inserts (XbaI: ˜8.5-kb, ˜8.5-kb, ˜1.45-kb; ClaI/XhoI: ˜10.0-kb, and ˜5.1-kb) were also made to orient and connect these HindIII fragments.

A Southern blot containing the HindIII-digested genomic DNA samples from the wild-type parental line (BGL2-GUS) and the three npr1 mutants was examined with probes generated from HindIII fragments made from the cosmid clone 21A4-P5-1. No significant difference in the restriction patterns was observed between the wild-type and all three npr1 allelic mutants. Therefore, it is unlikely that these mutants carried a substantial deletion in the NPR1 gene.

DNA fragments covering the 7.5-kb region were used to detect transcripts on a blot containing the polyA mRNAs made from four-week-old plants of the wild-type parental line and of the three npr1 allelic mutants seventy-two hours after treatment of the plants with H₂O or 0.65 mM INA and 2 mM SA. The polyA mRNA samples were prepared using Dynabeads (Dynal, Inc., Lake Success, N.Y.) from seventy-five micrograms of total RNA according to the protocol provided by Dynal. From this analysis, only one ˜2.0-kb mRNA was detected in the 7.5-kb region using probes made from the 0.5-kb and the adjacent 1.96-kb HindIII fragments. This mRNA represented a putative transcript of the NPR1 gene. In addition, the intensity of this transcript was about two-fold higher in the INA/SA-induced samples compared to the H₂O-treated controls as measured by a PhosphorImager and ImageQuant (Molecular Dynamics, Sunnyvale, Calif.). Thus, the expression of this transcript believed to represent mRNA of the NPR1 gene was induced by INA/SA treatment. No significant difference in the pattern of expression was discovered between the wild-type and three npr1 mutant alleles on this polyA RNA blot.

Sequence Analysis of the NPR1 Gene

The initial sequencing analysis was carried out using pBluescript SK⁺ clones of the five HindIII fragments as templates. The template DNA samples were prepared using Qiagen Plasmid Mini Kits (Qiagen Inc., Chatsworth, Calif.), and 0.6 μg of the template was used for each sequencing reaction and analyzed by an ABI automated sequencer.

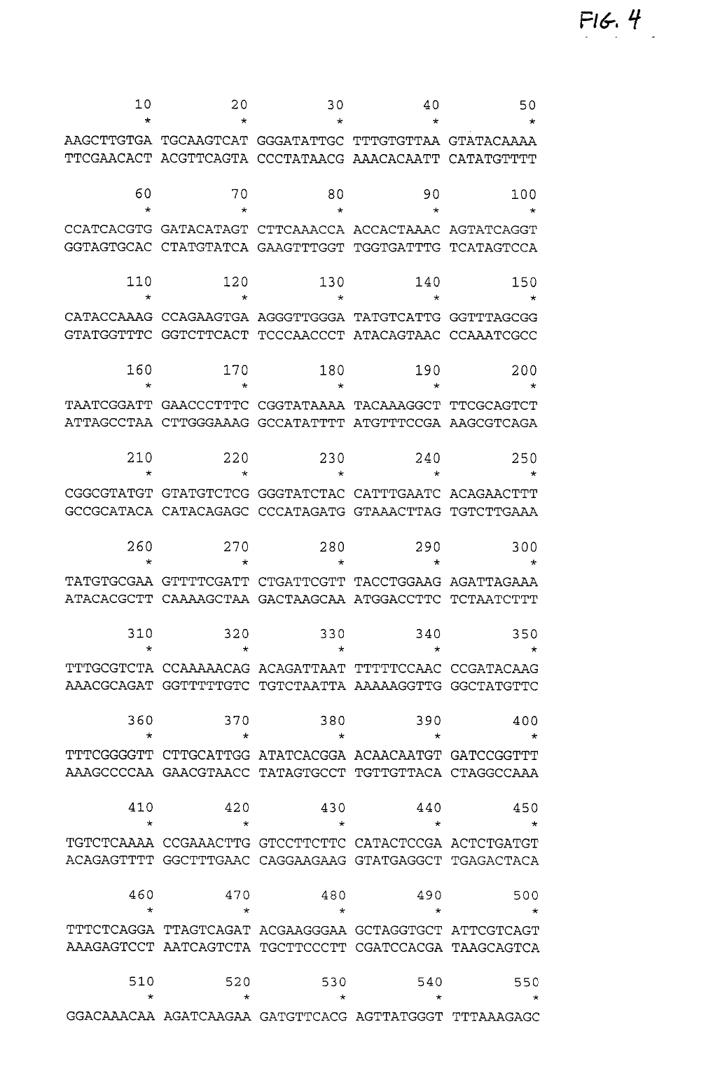

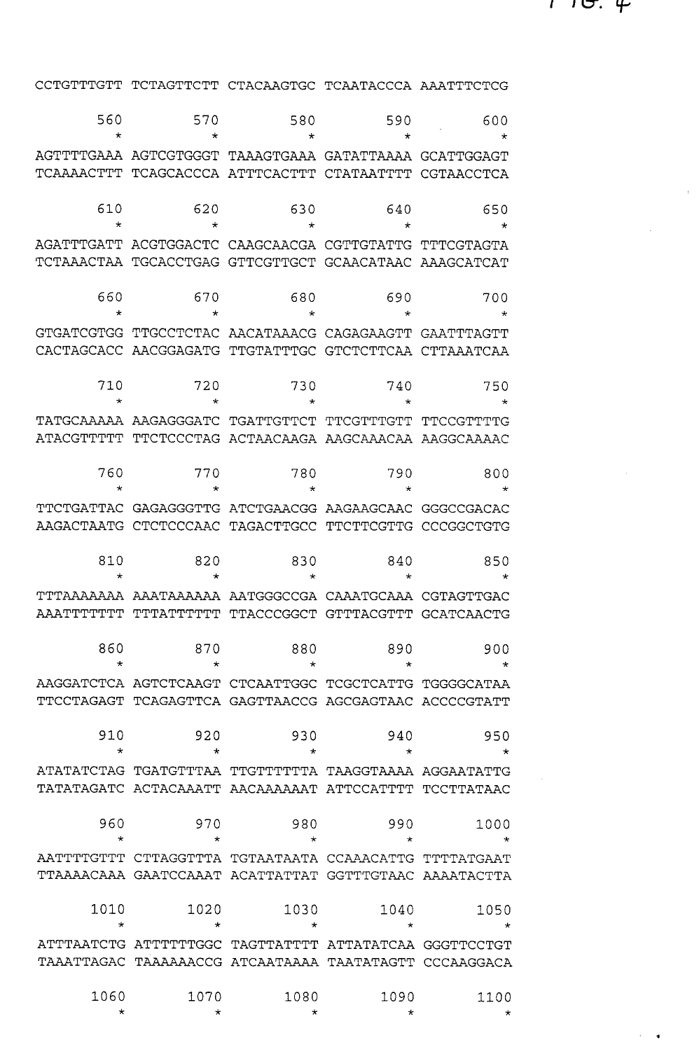

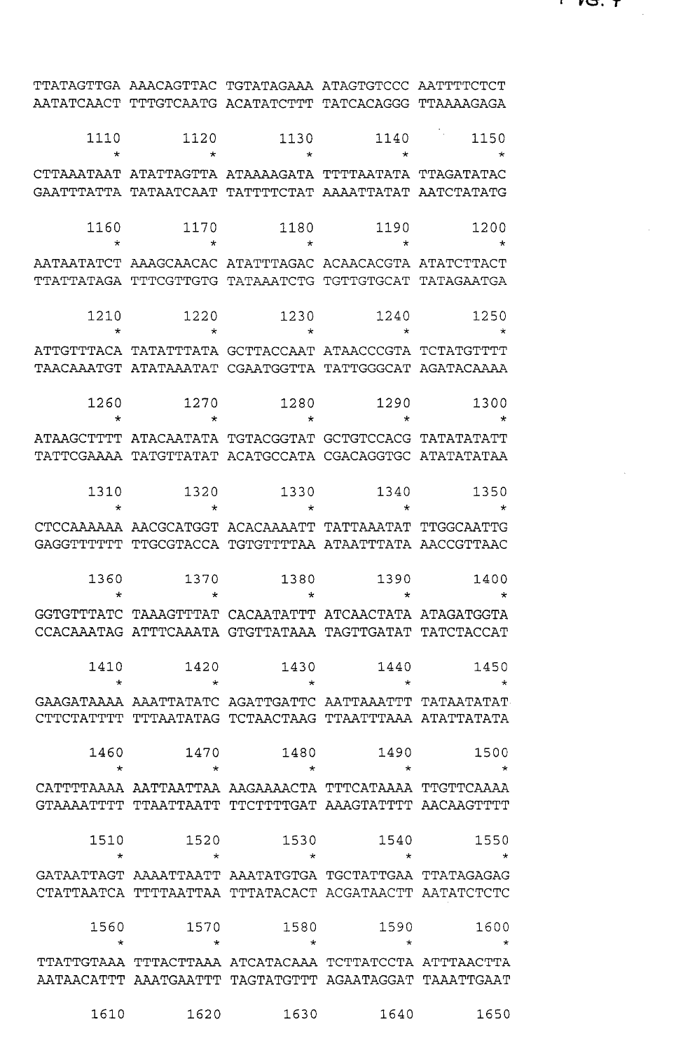

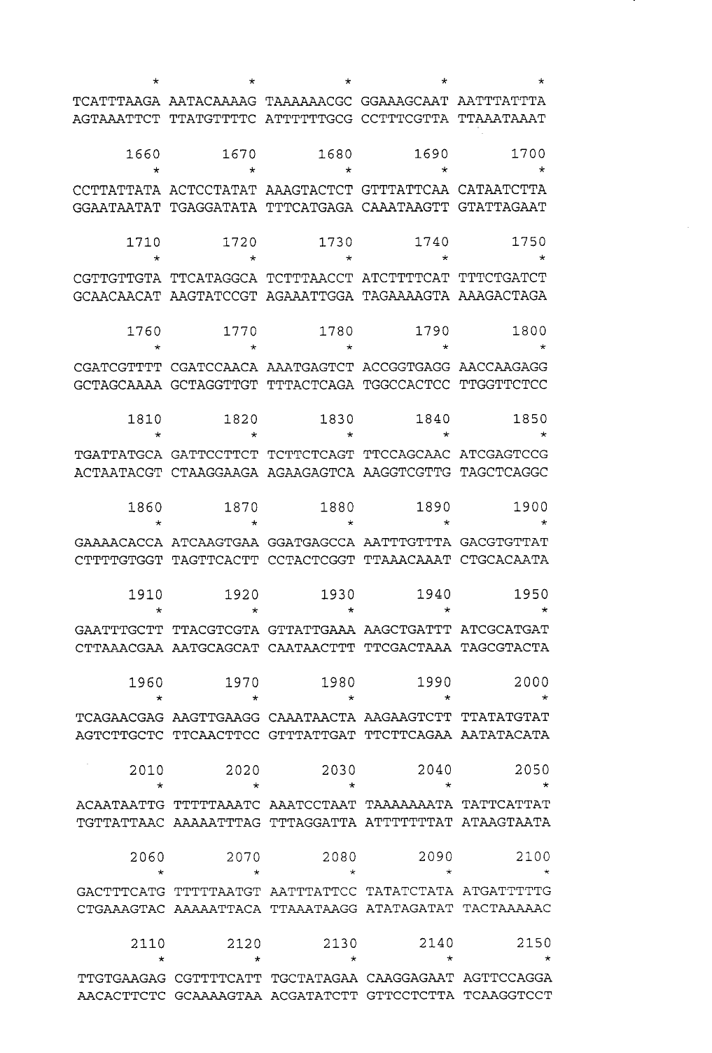

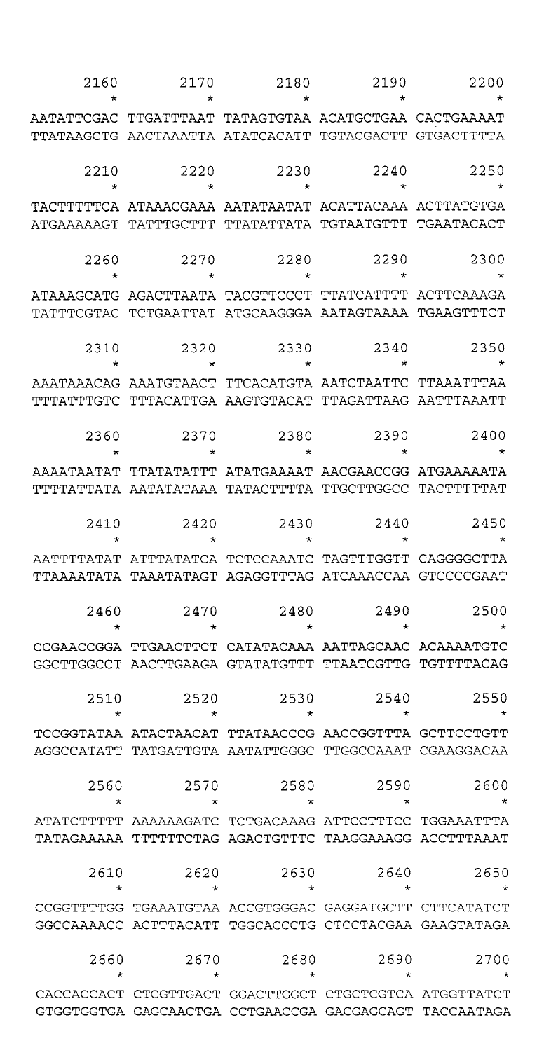

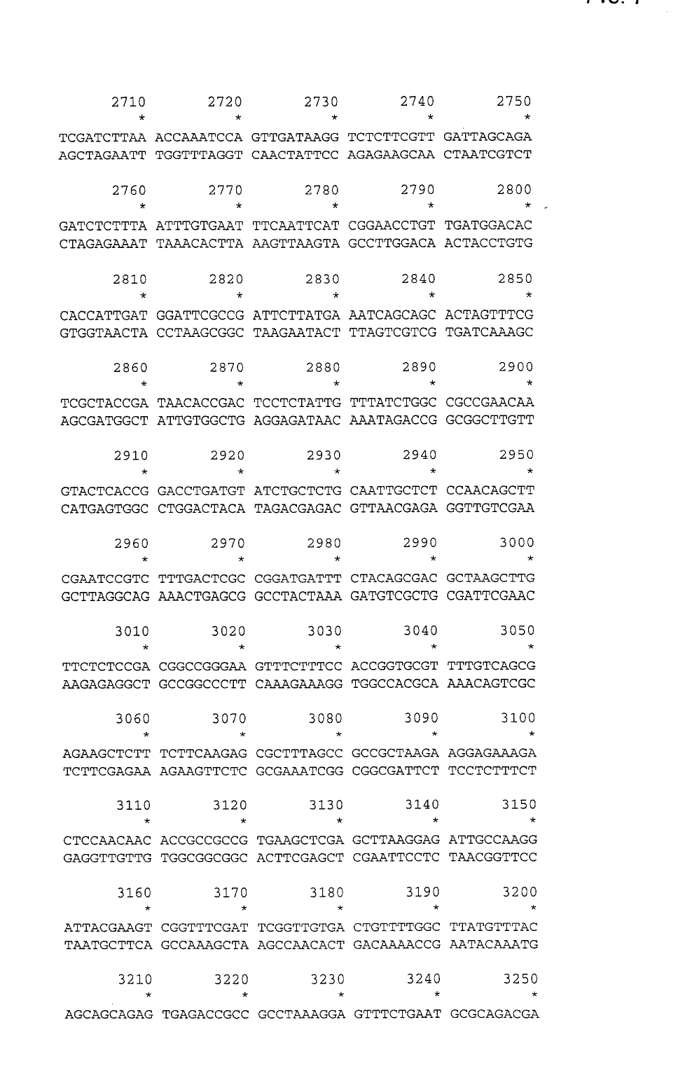

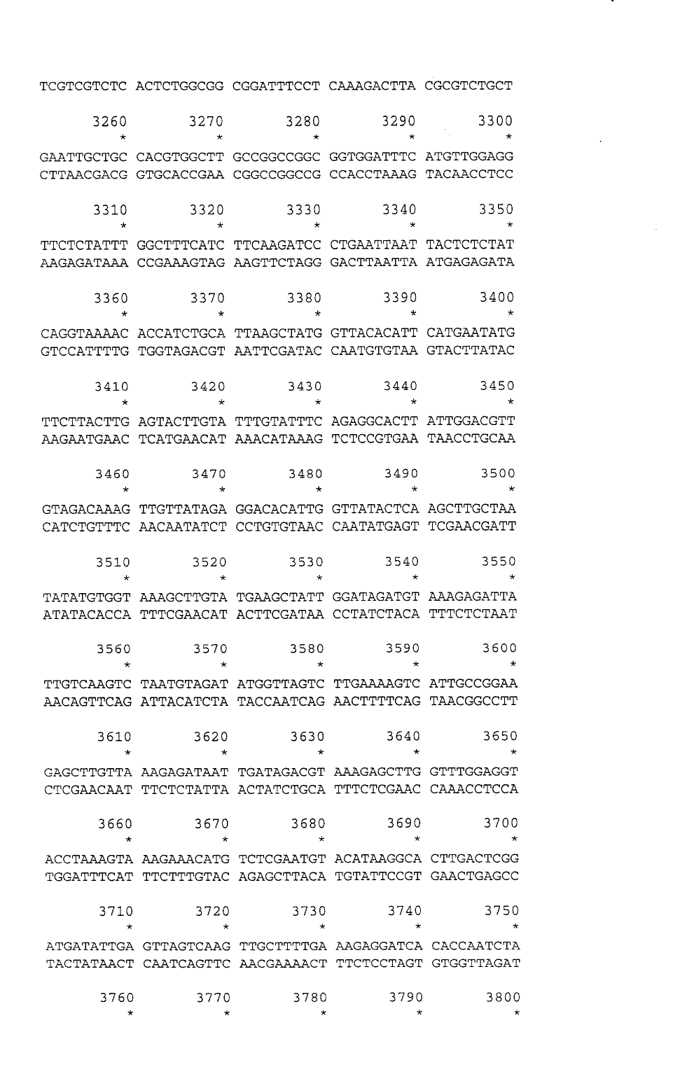

M13-20 and M13 reverse primers were used to initiate the sequencing reactions of the HindIII fragments. Various restriction enzymes were then used to generate deletions in these HindIII subclones to analyze sequences more distal to the ends of the fragments. In addition, primers were designed to perform primer walking. The relative positions of these HindIII fragments were determined and gaps between these fragments were filled by sequencing analyses using XbaI-subclones of cosmid 21A4-P5-1 as templates. The sequence data were analyzed to identify restriction enzyme sites, to perform sequence alignment and to search for open reading frames using standard DNA analysis software (DNA Strider 1.1, MacVector 4.0.1, and GeneFinder). Using this software only one putative gene was found. Sequence data were also compared to the TIGR Arabidopsis thaliana DataBase (http://www.tigr.org/tdb/at/at.html). The results of this study identified an expression sequence tagged (EST) clone that showed homology with a portion of the 1.96-kb fragment. This portion of the 1.96-kb fragment was also identified as part of the gene recognized using GeneFinder software. The nucleotide sequence of the 7.5-kb genomic region encoding the NPR1 gene product is shown in FIG. 4.

Isolation of NPR1 cDNA Clones

A cDNA library that was constructed by Dr. Katagiri (and described in detail in Mindrinos et al., Cell 78:1089-1099, 1994) was screened using the 1.96-kb HindIII fragment as a probe. Bacterial cells (E coli DH10B; GIBCO BRL, Gaithersburg, Md.) containing cDNAs made from the aerial parts of one-month old wild-type Arabidopsis plants in vector pKEx4tr were plated (60,000 cfu/plate) on LB medium containing 100 μg/mL ampicillin, and the plates were incubated at 37° C. for four and one-half hours. Colonies were lifted onto Colony/Plaque Screen membranes (NEN Research Product; Boston, Mass.), and then the membranes were placed onto an LB plate, with the colony side up. Both plates were incubated at 30° C. for twelve hours. The membranes were autoclaved for one minute to lyse the cells and fix the DNA to the membrane. Hybridization was performed at 42° C. in a solution containing 10% dextran sulfate, 50% formamide, 6X SSC, 5X Denhardt's, and 1% SDS; and the membranes were washed twice at 65° C. in 2X SSC and 1% SDS. The positive colonies were purified through secondary and tertiary screens using identical conditions. One positive cloned was subsequently identified and designated pKExNPR1.

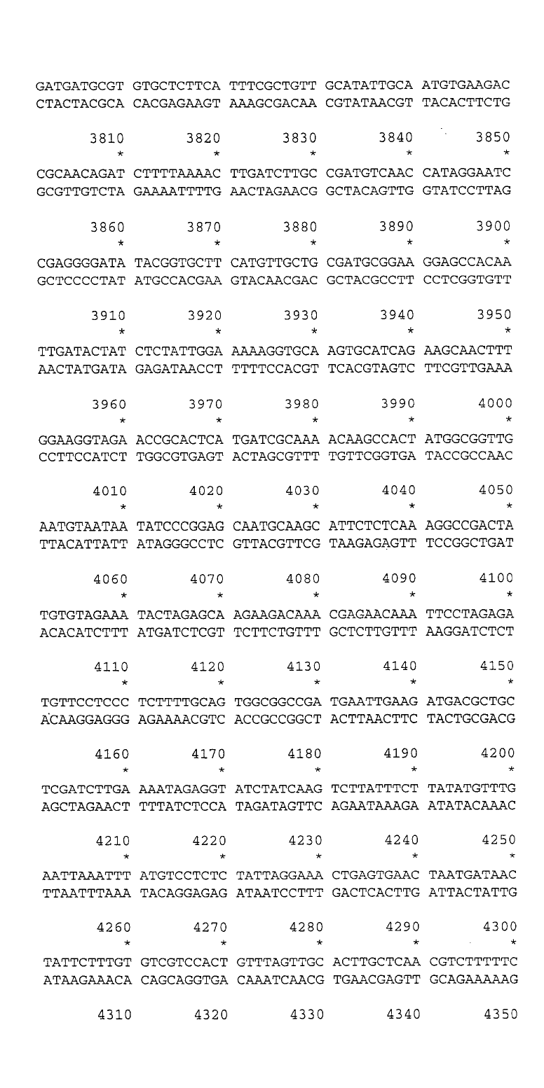

The cDNA inserts were excised from the vector using restriction enzymes EcoRI and SacI. Southern analysis was performed using probes made from the 1.96-kb (the 3′-end of the open reading frame) and the 0.5-kb (the 5′-end of the open reading frame) HindIII fragments to confirm homology of the cDNA clones. The nucleic acid sequence (SEQ ID NO:2) and deduced amino acid sequence (SEQ ID NO:3) of the acquired resistance protein termed NPR1 from Arabidopsis thaliana encoded by the 2.1-kb cDNA is shown in FIG. 5. Sequence analysis revealed that this cDNA contained sequences corresponding to those identified in the EST clone and deduced using the Gene Finder software.

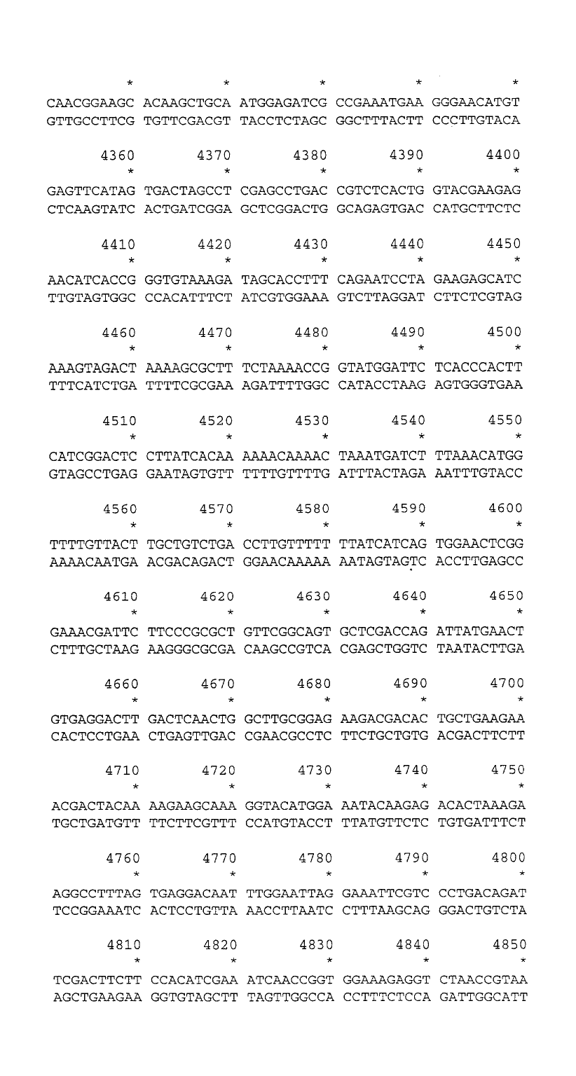

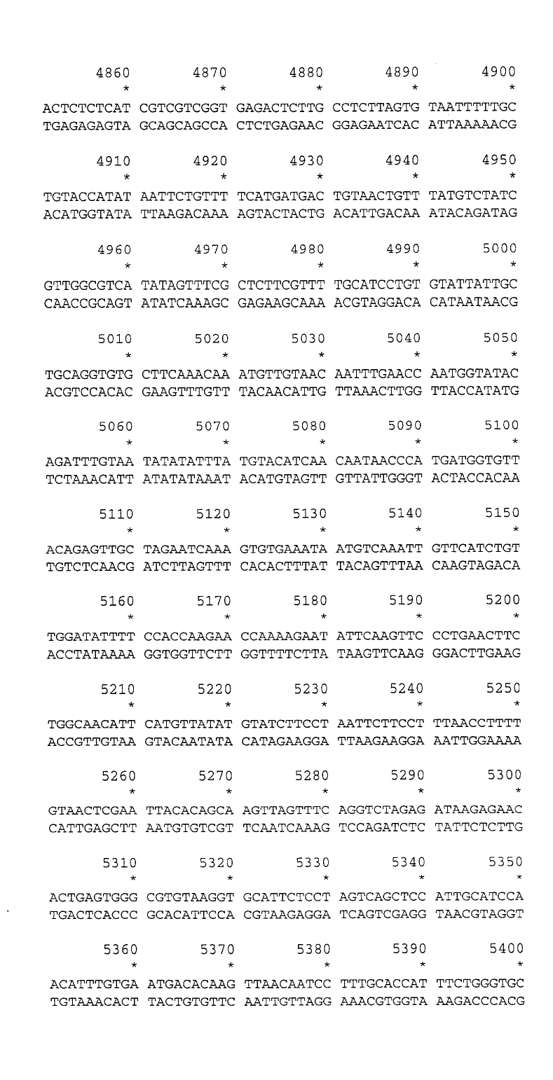

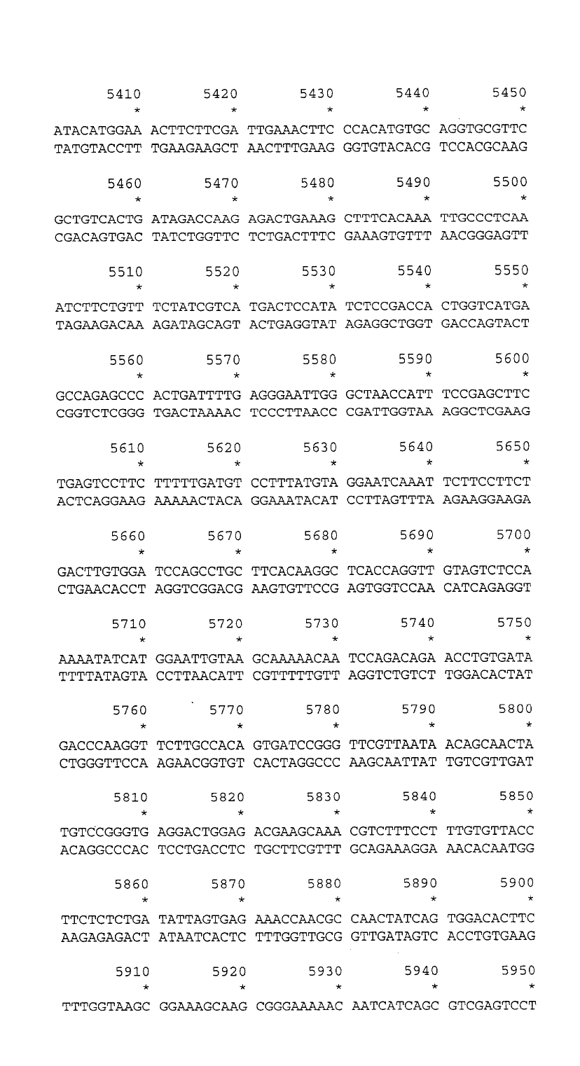

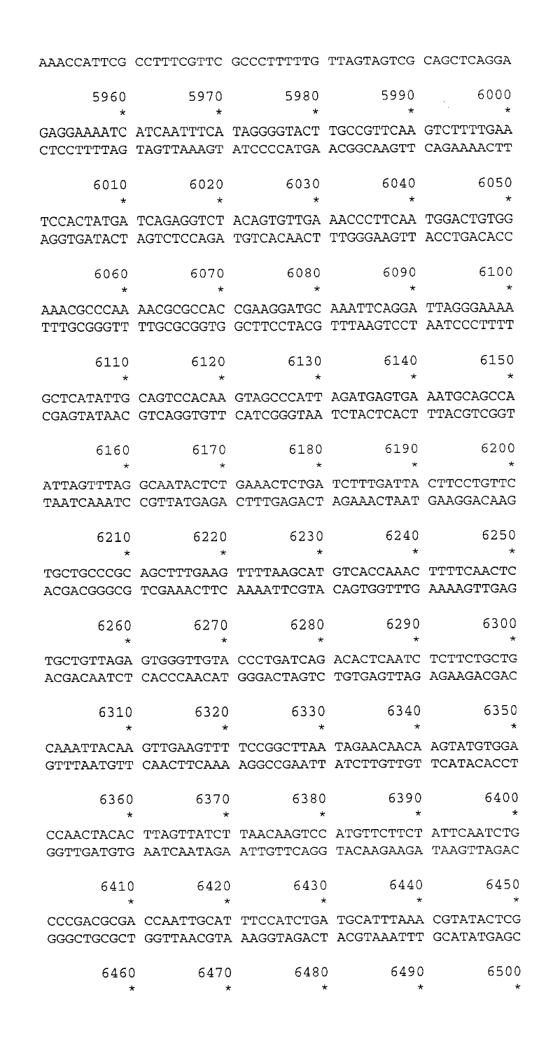

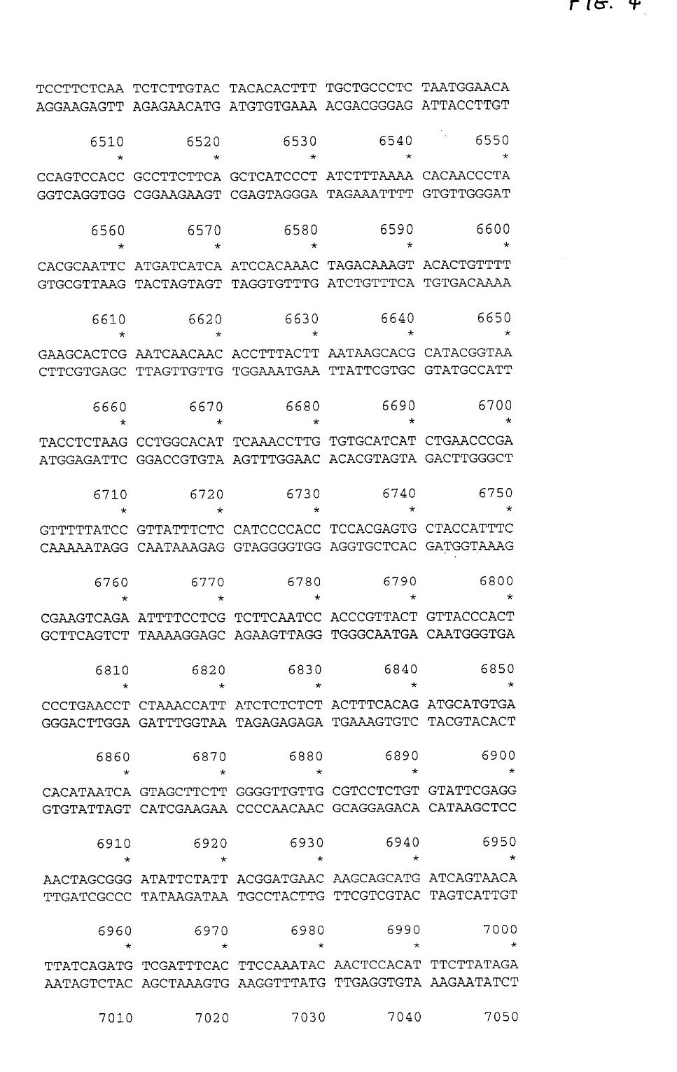

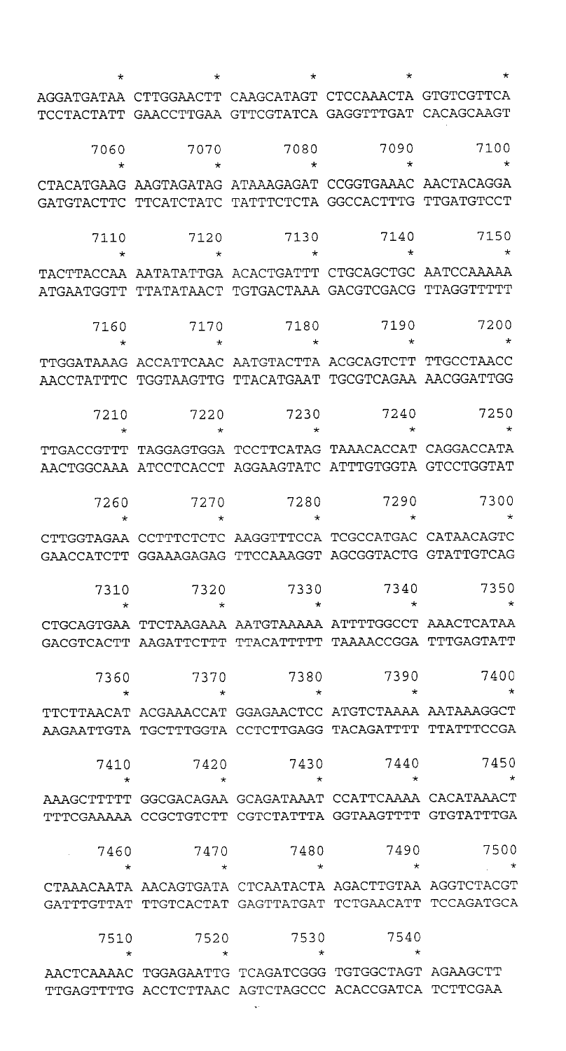

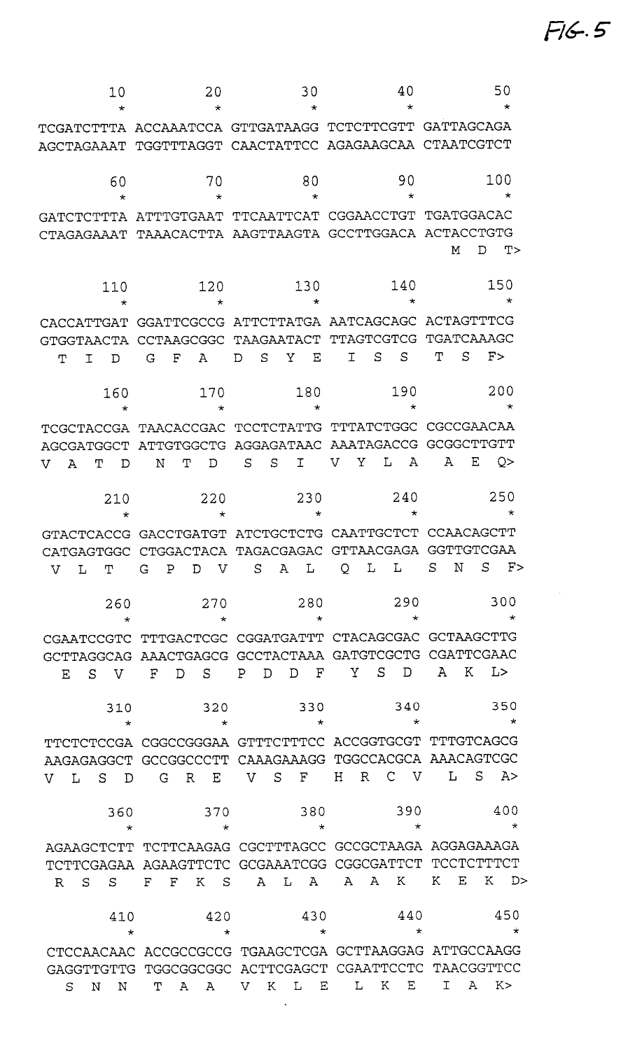

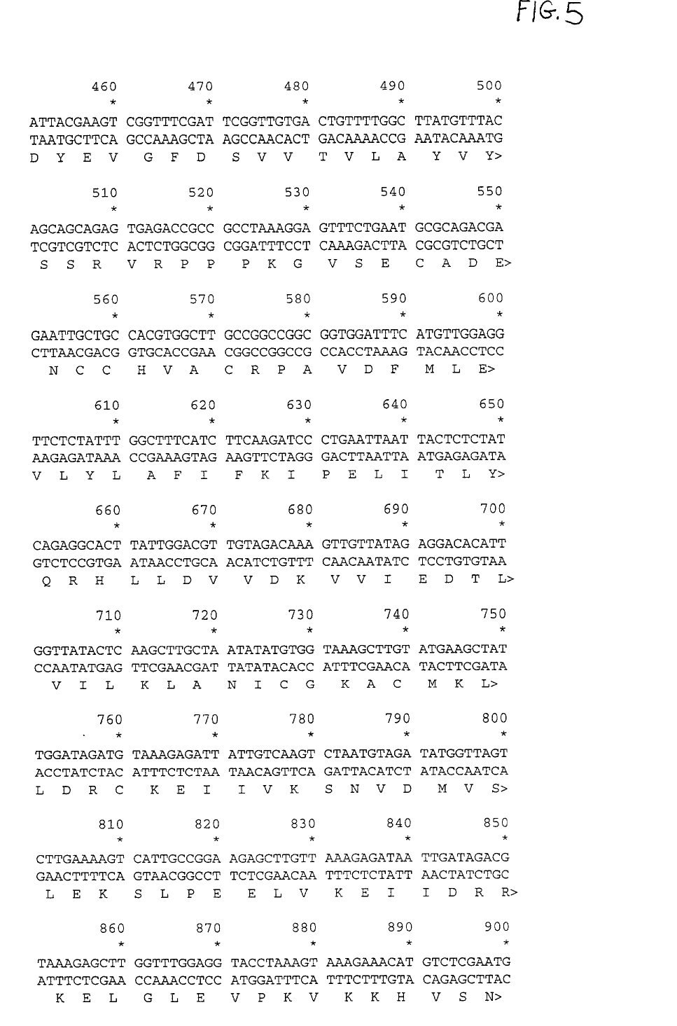

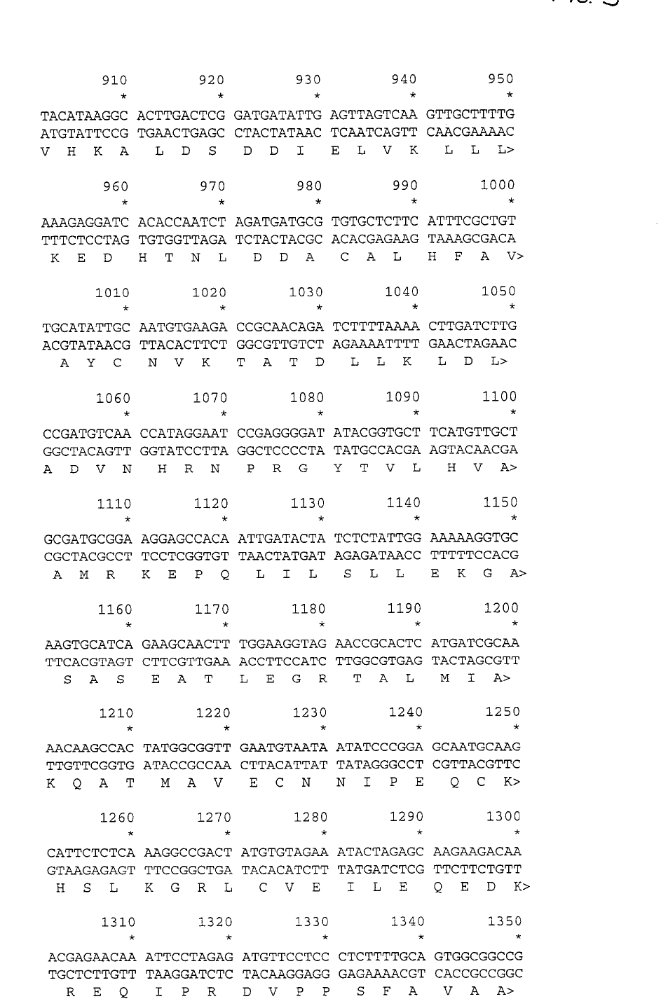

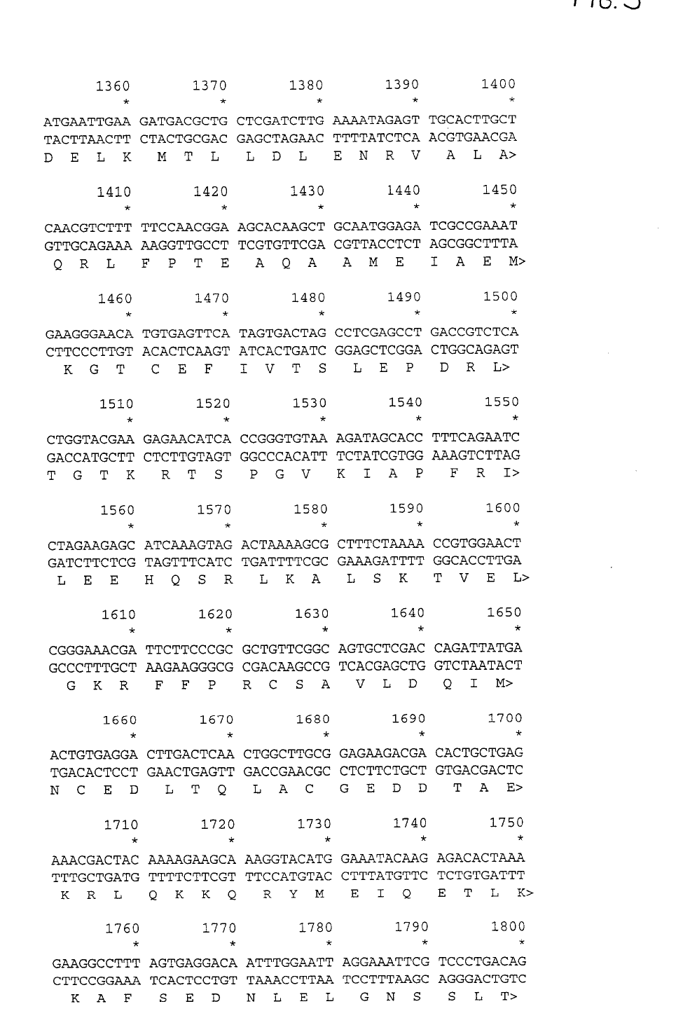

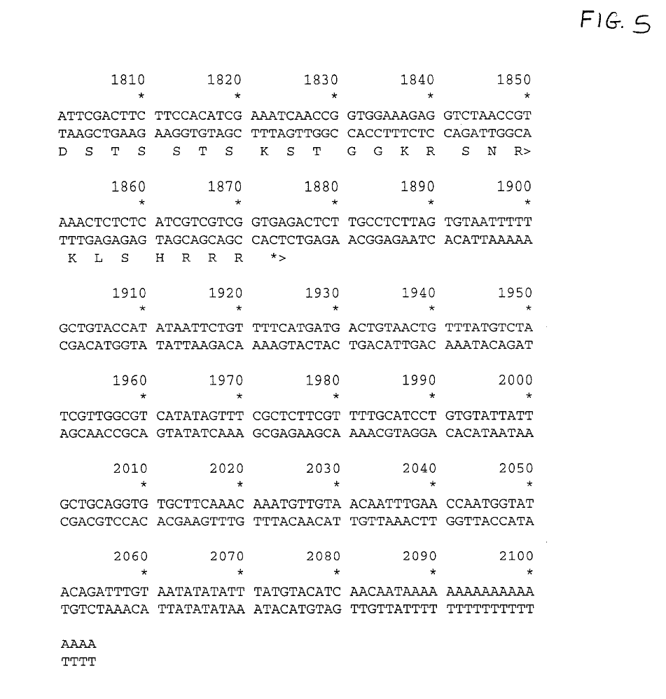

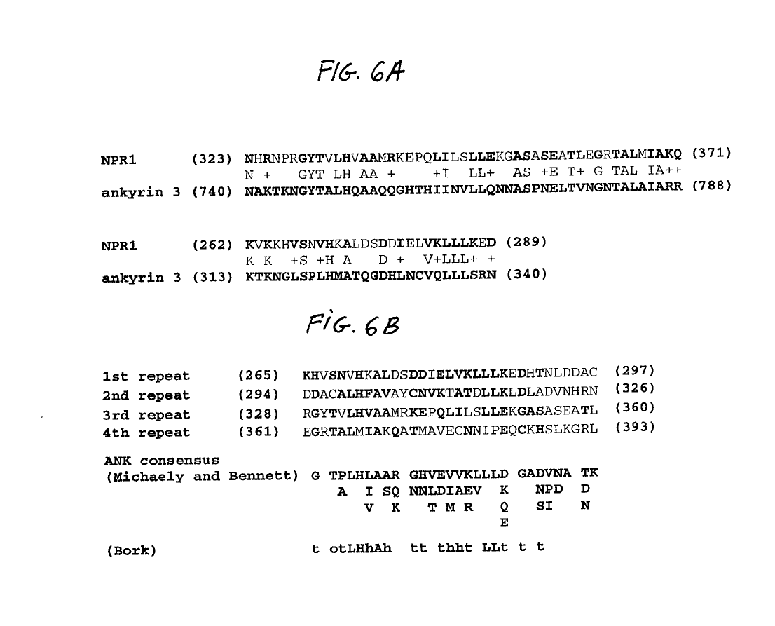

The cDNA sequence was analyzed using the BLAST sequence analysis program. This analysis revealed that the NPR1 protein shared significant homology with ankyrin, including the region identified as the ankyrin-repeat consensus. In particular, as shown in FIG. 6A, the NPR1 sequence contains two regions with significant homology to the mammalian ankyrin 3 gene. The sequence identities between NPR1 (amino acids 323-371 and 262-289) and ANK3 (amino acids 740-788 and 313-340) are 42% and 35%, respectively, and the sequence similarities are 59% and 57%, respectively. This ankyrin-repeat consensus has been identified in a diverse array of proteins including transcription factors, cell differentiation molecules, structural proteins, and proteins with enzymatic and toxic activities. This motif has been shown to function by mediating protein interactions.

Using the consensus sequence defined by Michaely and Bennett (Trends in Cell Biology 2:127-129, 1992) and Bork (Proteins: Structure, Function, and Genetics 17:363-374, 1993), two additional ankyrin repeats were identified in NPR1; these are shown in FIG. 6B.

In addition, using the MacVector program, a 17 amino acid motif of G-protein coupled receptors (MKGTCEFIVTSLEPDRL, FIG. 5, SEQ ID NO:21) has been found in the NPR1 protein (Science 244:569-572, 1989).

The NPR1-determined Resistance is Dosage Dependent

The ability of NPR-1 to confer disease resistance was evaluated in transgenic plants as follows. The NPR1 cDNA sequence (FIG. 5; SEQ ID NO:2) driven by the constitutive CaMV 35S promoter was transformed into Arabidopsis ecotype Columbia according to standard methods. In the resulting T₃ lines homozygous for the 35S-NPR1 transgene, the expression of the NPR1-regulated PR-1 gene, NPR1 mRNA, and NPR1 protein were measured to identify those lines exhibiting high (Co1NPR1H), medium (Co1NPR1M), and low (Co1NPR1L) levels of NPR1 expression. Table 1 shows the results of evaluating the relative levels of PR-1, NPR1 mRNA, and NPR1 protein concentrations.

TABLE 1
Characterization of 35S-NPR1 Transgenic Lines

| | PR-1 | NPR1 | NPR1 |
|---|---|---|---|
| Genotype | (INA)^{a} | (mRNA)^{b} | (Protein)^{c} |
| Col | 1.00 | 1.00 | 1.00 |
| Col-L1 | 0.41 | 6.92 | 0.04 |
| Col-L2 | 0.54 | 6.90 | <0.04 |
| Col-M1 | 1.73 | 9.20 | 1.40 |
| Col-M2 | 1.80 | 9.50 | 1.40 |
| Col-H1 | 2.60 | 17.80 | 1.60 |
| Col-H2 | 2.74 | 27.90 | 3.00 |

^{a}The relative levels of PR-1 were measured by an RNA blot analysis in the 35S-NPR1 transgenic lines grown on plates containing 0.1 mM INA.
^{b}The relative levels of NPR1 mRNA were measured by a polyA + RNA blot.
^{c}The relative NPR1 protein concentrations were measured by ELISA using NPR1 polyclonal antibodies.

From these experiments, two lines of transformants were identified that had significantly lower NPR1 protein levels (but not mRNA levels) than the wild-type parent. This, however, was not unexpected because overexpression of a transgene in plants often leads to co-suppression of the transgene as well as the corresponding endogenous gene (Baulcombe, The Plant Cell, 8:1833, 1996).

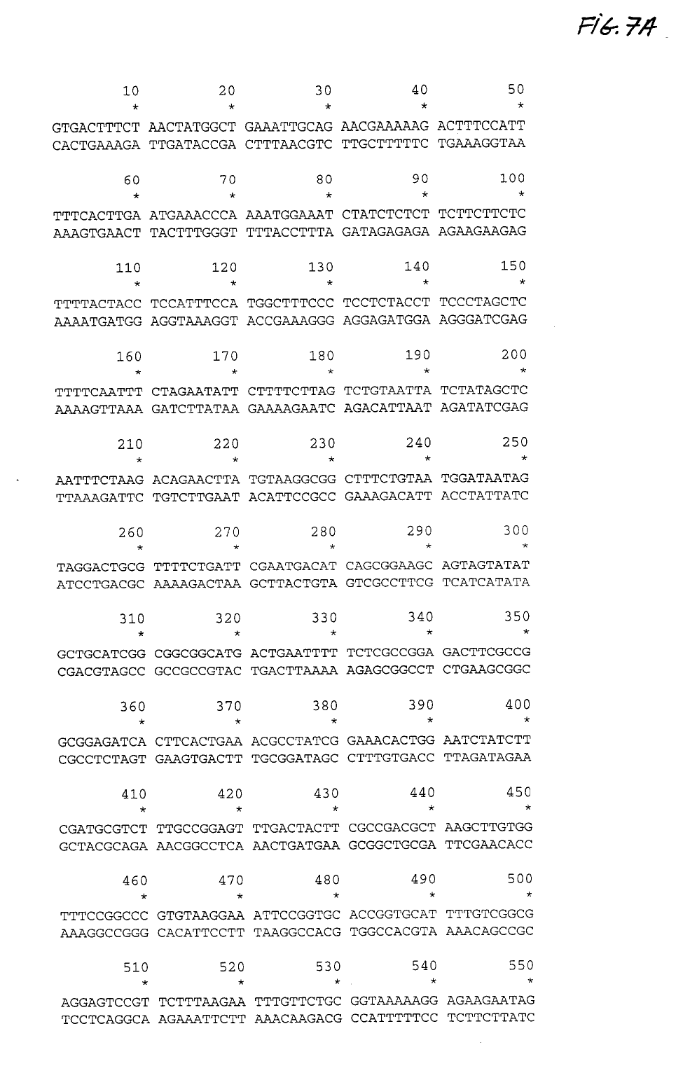

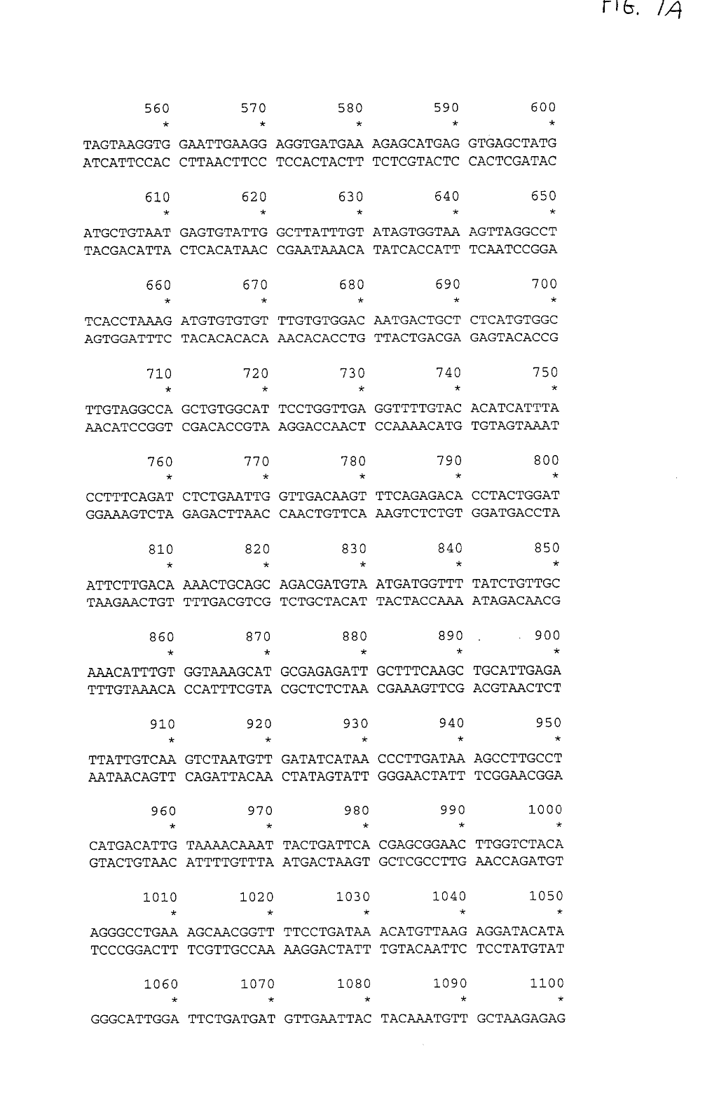

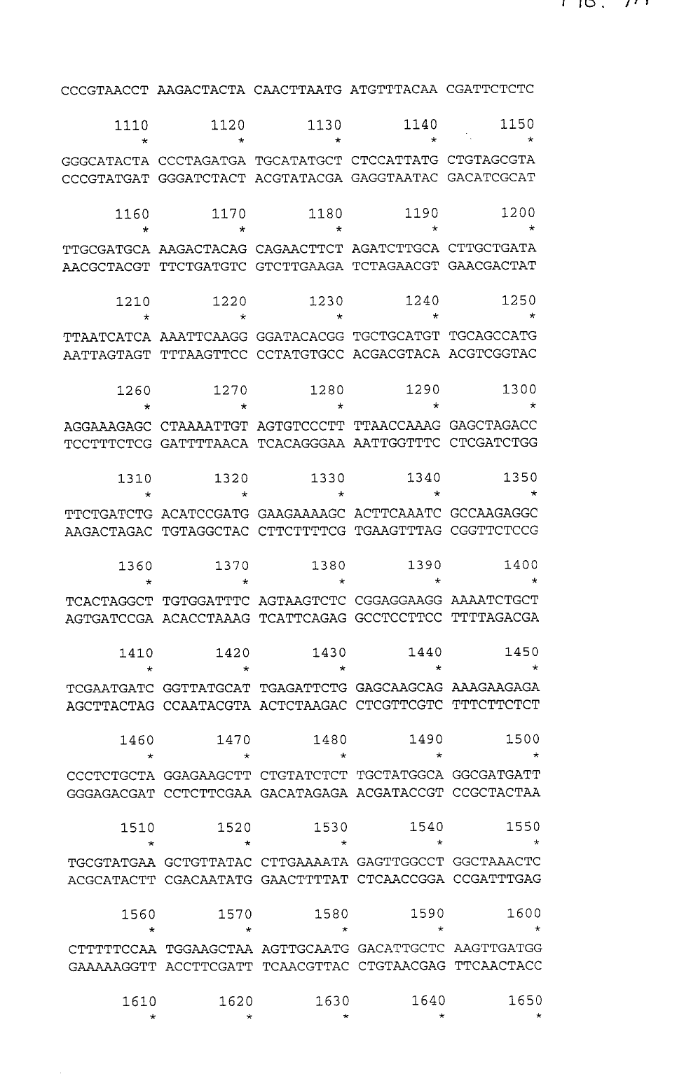

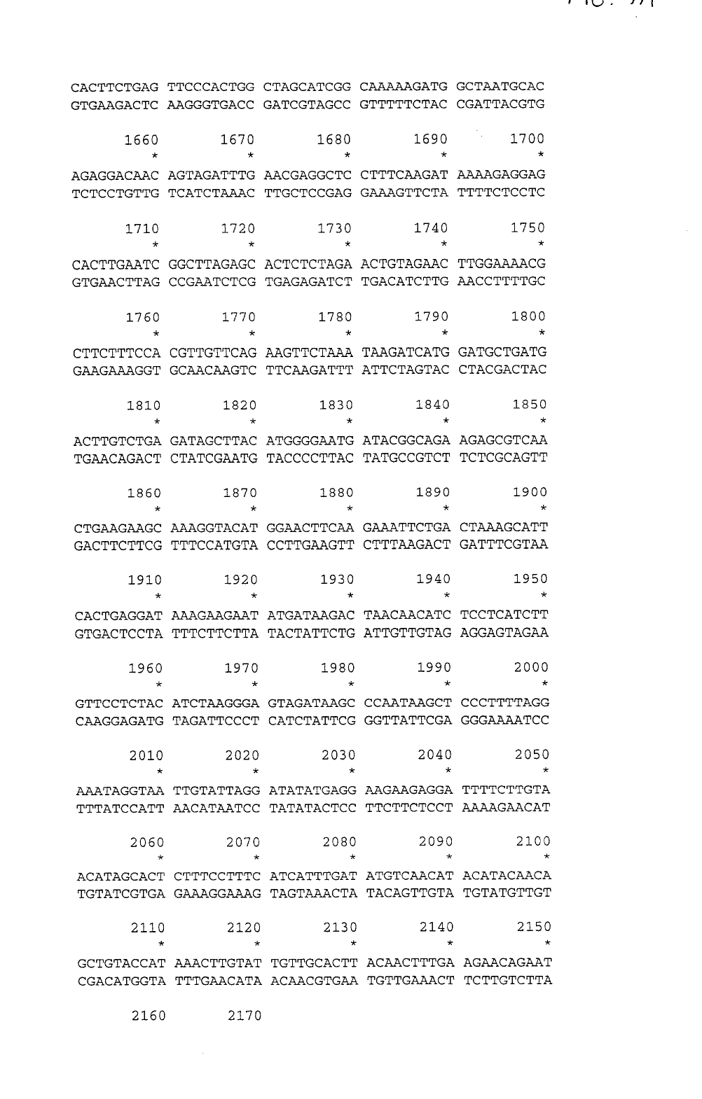

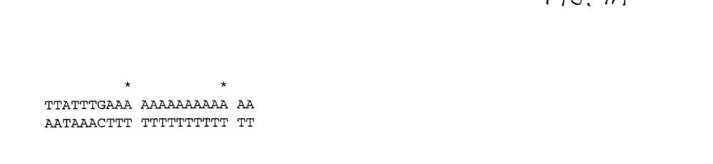

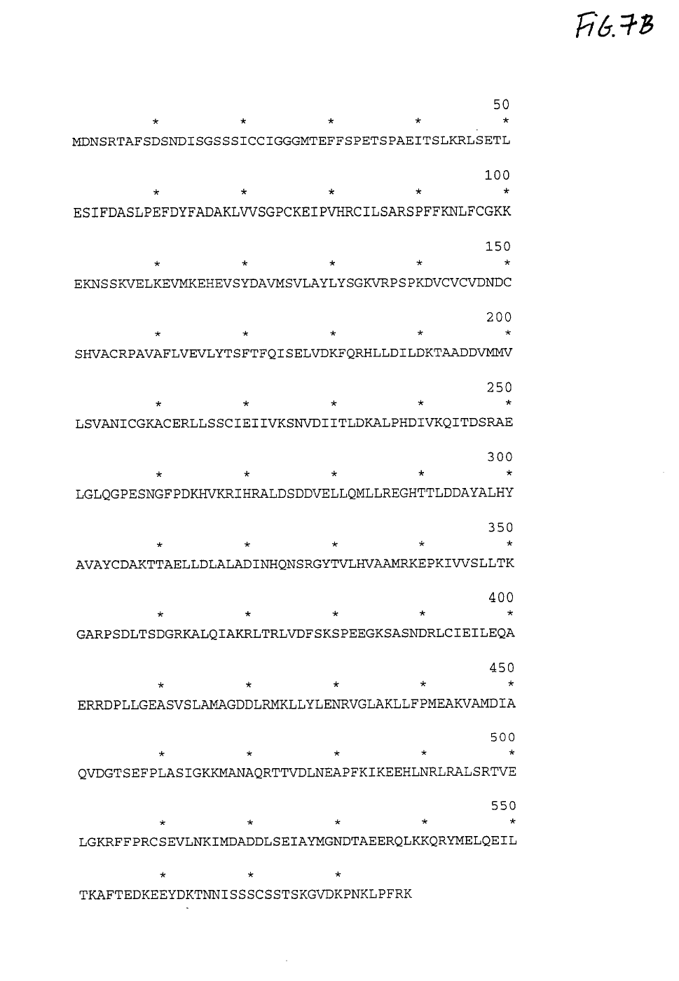

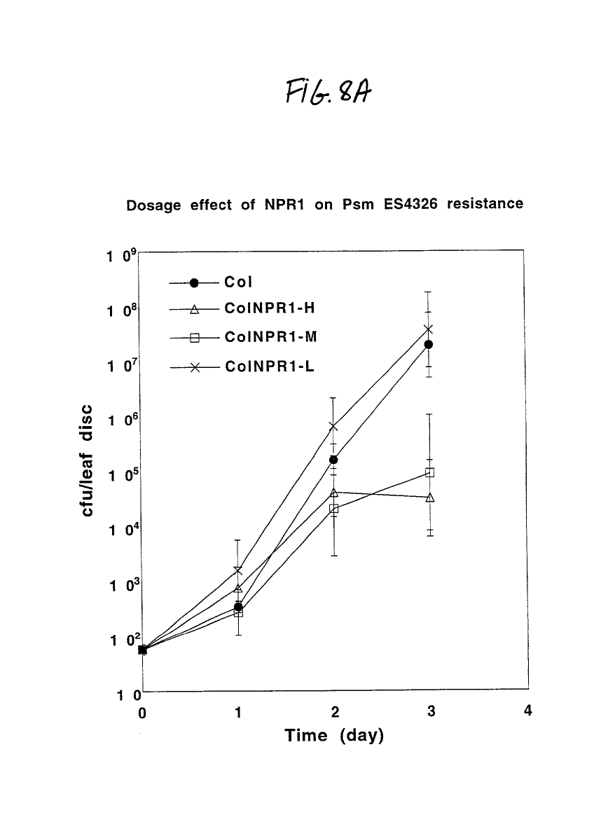

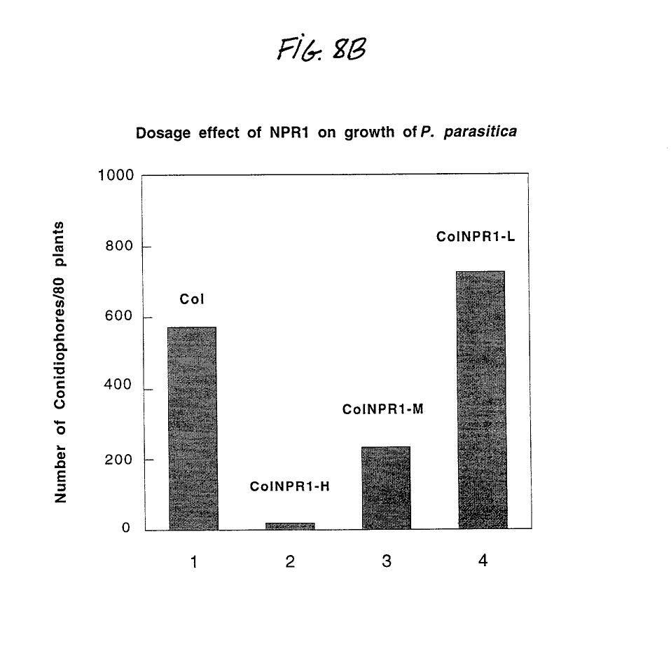

The high-, medium-, and low-expressing 35S-NPR1 transgenic lines were next subjected to infection by the bacterial pathogen Pseudomonas syrinigae pv maculicola ES4326 and the fungal pathogen Peronospora parasitica NOCO2 according to standard methods. The results of these experiments are shown in FIGS. 8A and 8B, respectively. In the absence of SAR induction, the high- and the medium-expressing 35S-NPR1 transgenic lines showed significantly increased resistance to both bacterial and fungal pathogens while the low-expressing transgenic lines displayed reduced tolerance to the pathogens as compared to the wild-type. Together, these results showed that NPR1 was a positive regulator of SAR, and that the NPR1-determined resistance was dosage dependent; overexpression of the NPR1 protein enhanced resistance whereas underexpression led to reduced tolerance to infection.

NPR1 is Translocated to the Nucleus Upon SA Induction

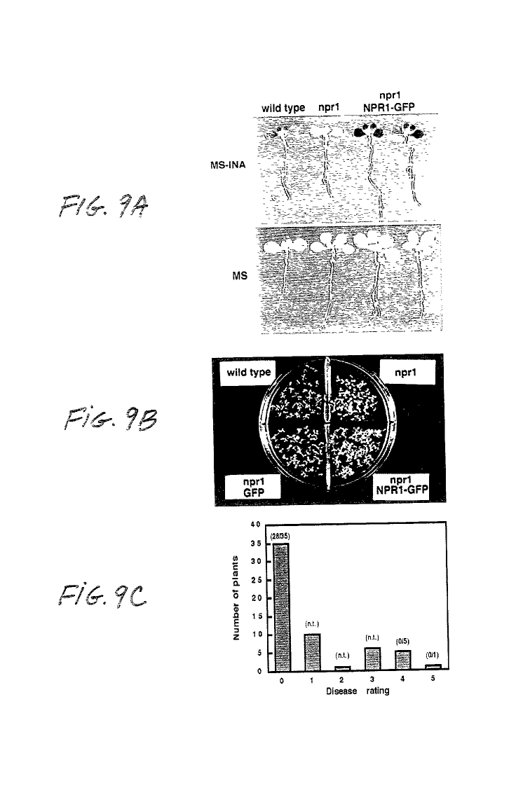

To elucidate the induction mechanism and the molecular function of the protein, the subcellular localization of NPR1 was determined by using standard reporter gene fusion construct analysis. The green fluorescent protein (GFP) gene was fused to the carboxyl end of the NPR1 cDNA driven by the constitutive CaMV 35S promoter, and the 35S-NPR1-GFP construct was used to transform npr1 mutants, npr1-1 and nprl-2, according to standard methods. In the resulting transgenic lines, the NPR1-GFP transgene was found to complement all the npr1 mutant phenotypes; namely, the lack of SA- or INA-induced PR gene expression, the reduced tolerance to exogenous SA, and the lack of SA- or INA-induced resistance to pathogens (FIGS. 9A-9C). Transgenic lines expressing the GFP alone (designated 35S-mGFP), exhibited no complementing activity (FIG. 9B). In addition, the presence of the NPR-GFP transgene was found to restore both inducible BGL-GUS expression and resistance to P. parasitica as shown in FIGS. 9A and 9C, respectively. These experiments therefore showed that the NPR1-GFP was biologically active and that the subcellular localization of NPR1-GFP should reflect that of the endogenous NPR1 protein.

To examine the subcellular localization of the NPR1 protein, the 35S-NPR1-GFP and 35S-mGFP transgenic lines were grown in MS medium in the presence or absence of the SAR-inducing chemicals SA or INA. Eleven-day-old seedlings were subsequently examined using confocal microscopy to detect localization of NPR1-GFP and mGFP. As shown in FIG. 10, the 35S-NPR1-GFP seedlings grown on MS showed low levels of GFP throughout the mesophyll cells and strong GFP fluorescence in the nuclei of the guard cells. Upon induction by SA or INA, NPR1-GFP was detected exclusively in the nuclei of both the mesophyll cells and the guard cells. In the 35S-mGFP transformants, green fluorescence was detected in the cytoplasm as well as in the nuclei, and SA and INA treatments had no effect on the localization of the protein. These results indicated that NPR1 was localized in the cytoplasm in the mesophyll cells, and that upon induction the NPR1 protein was transported into the nucleus resulting in PR1 gene expression and resistance. In the guard cells, the NPR1 protein was localized in the nuclei even without an SAR induction, an intriguing observation because constitutive activation of defense mechanisms in these cells may be necessary to fend off microbial pathogens from gaining entry into the plant through stomata. Since mGFP alone showed no induced nuclear translocation, the nuclear transport of the NPR1-GFP fusion must be directed by a signal in NPR1. Consistent with this, the following two potential nuclear localization sequences (NLS's) were found in NPR1:

252 RRKELGLEVPKVKK 265 (SEQ ID NO:22); and

541 KKQRYMEIQETLKK 554 (SEQ ID NO:23).

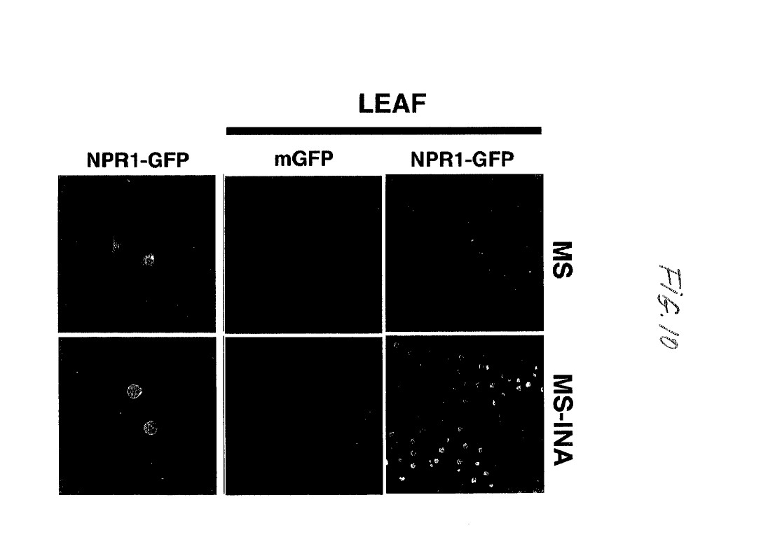

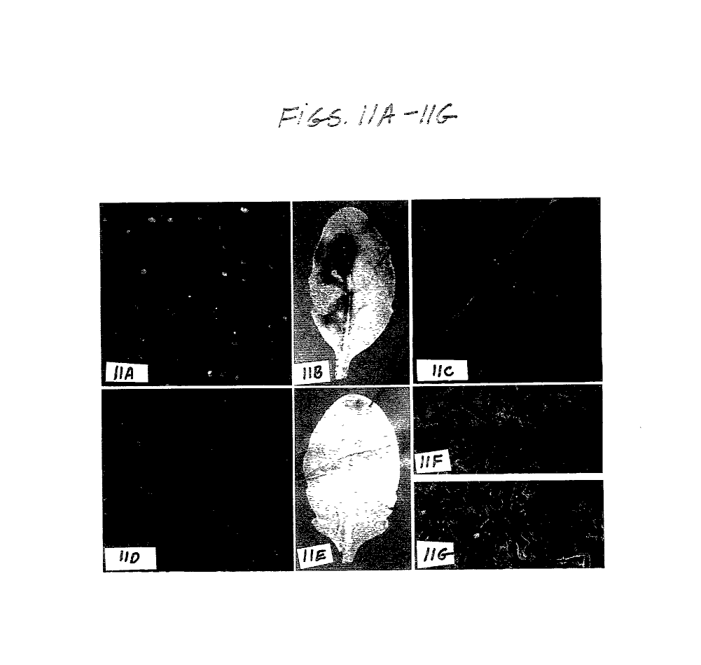

Significantly, nuclear translocation in tissues infected by the virulent pathogen Psm ES4326 was also observed (FIG. 11A). This pattern of induction was also observed to coincide with the pattern of PR gene expression observed in plants after infection (FIG. 11B).

Characterization of npr Mutations

To further characterize the NPR1 gene, the mutations in npr1-1, npr1-2, npr1-3, and npr1-4 were identified by DNA sequencing. The mutant npr1-4 is a new npr1 allele that was identified in the Col-0 (BGL2-GUS) background based on its enhanced susceptibility to Psm ES4326. Each mutant allele was found to contain a single base-pair change. The npr1-1, npr1-2, npr1-3, and npr1-4 alleles respectively altered the highly conserved histidine (residue 334) in the third ankyrin-repeat consensus to a tyrosine, changed a cysteine (residue 150) to a tyrosine, introduced a nonsense codon (residue 400) that should result in a truncated protein lacking 194 amino acids of the C-terminal end of the protein, and destroyed the acceptor site of the third intron junction. All of these point mutations are GC to AT transitions, consistent with the mode of action of the mutagen, ethyl-methanesulfonate (EMS), used for the generation of these mutations.

Genetic Analysis of the Plant Defense Response Using Arabidopsis thaliana

Although biochemical studies have played an important role in elucidating the general features of the plant defense response, the complexity of the defense response limits the utility of biochemical analysis in determining the importance of particular defense responses or enzymes in conferring resistance to pathogens. Isolation of plant defense-response mutants not only helps elucidate the roles of known pathogen-induced responses in combating particular pathogens, but also facilitates the identification of plant defense mechanisms not already correlated with a known biochemical or molecular genetic response. With the development of well-characterized hostpathogen systems involving the model plant Arabidopsis thaliana as the host as described herein, comprehensive genetic analysis of acquired resistance responses is made possible.

All of the major features of the plant defense response that have been observed in crop plants have also been observed in Arabidopsis-pathogen interactions. For example, several resistance gene-avr gene interactions have been identified for both bacterial and fungal pathogens of Arabidopsis (Bisgrove et al., Plant Cell 6:927-933, 1994; Holub et al., Mol. Plant-Microbe Interact. 7:223-239, 1994; Kunkel et al., Plant Cell 5:865-875, 1993; Yu et al., Mol. Plant-Microbe Interact. 6:434-443, 1993). Moreover, all of the important features of SAR have been observed in Arabidopsis (Uknes et al., Plant Cell 4:645-656, 1992; Uknes et al., Mol. Plant-Microbe Interact. 6:692-698, 1993). Importantly, the power of Arabidopsis genetic analysis has recently been used to help identify a variety of components of the Arabidopsis defense response to pathogen attack (Bent et al., Science 265:1856-1860, 1994; Bowling et al., Plant Cell 6:1845-1857, 1994; Cao et al., Plant Cell 6:1583-1592, 1994; Century et al., Proc. Natl. Acad. Sci. USA 92:6597-6601, 1995; Delaney et al., Proc. Natl. Acad. Sci. USA 92:6602-6606, 1995; Dietrich et al., Cell 77:565-577, 1994; Glazebrook and Ausubel, Proc. Natl. Acad. Sci. USA 91:8955-8959, 1994; Glazebrook et al., Genetics 143:973-982, 1996; Grant et al., Science 269:843-846, 1995; Greenberg and Ausubel, Plant J. 4:327-341, 1993; Greenberg et al., Plant J. 4:327-341, 1994; Mindrinos et al., Cell 78:1089-1099, 1994). Thus, the results described herein provide the basis for identifying genes that are involved in acquired disease resistance throughout the plant kingdom and are not limited to Arabidopsis.

Isolation of Solanaceous AR Genes

Using the Arabidopsis NPR1 cDNA sequence shown in FIG. 5 (SEQ ID NO:2), the isolation of AR homologs that are found in solanaceous plants (e.g., potato, eggplant, tomato, tobacco, petunia, and pepper) is readily accomplished using standard techniques.

For example, a Nicotiana glutinosa cDNA library was screened for the presence of an NPR1 homolog. The library was constructed in the lambda ZAP II vector from poly (A+)RNA isolated from Nicotiana glutinosa plants infected with tobacco mosaic virus (TMV) (Whitham et al., Cell 78: 1101-1115, 1994). Bacteriophage were plated on NZY media using XL-1 Blue host cells. Approximately 10⁶ plaques were screened by transferring the phage DNA onto positively charged nylon membrane (GeneScreen; DuPont-New England Nuclear) and probing with a random primed ³²P labeled probe that was prepared using the full-length Arabidopsis NPR1 cDNA as the template. Hybridization was performed at 37° C. in 40% formamide, 5X SSC, 5X Denhardt, 1% SDS, and 10% dextran sulfate. The filters were washed in 2X SSC for fifteen minutes at room temperature and 2X SSC, 1% SDS for thirty minutes at 37° C.

Two hybridizing clones were identified and purified. The pBluescript plasmids were excised using XL-1 Blue host cells and R408 helper phage. Restriction enzyme analysis indicated that the two positive clones contained inserts of approximately 3600 bp and 2100 bp. Restriction digests and sequence analysis indicated that the 3600 bp insert represented two independent cDNAs of 2100 bp and 1500 bp and that the two independently isolated 2100 bp cDNAs were identical. Both strands of the 2100 bp cDNA were sequenced using ³⁵S-dATP and the Sequenase sequencing kit (U.S. Biochemicals, Cleveland, Ohio). The nucleotide and amino acid sequences encoding the Nicotiana glutinosa NPR1 homolog are shown in FIG. 7A (SEQ ID NO:13) and FIG. 7B (SEQ ID NO:14), respectively.

Isolation of Other Acquired Resistance Genes

Any plant cell can serve as the nucleic acid source for the molecular cloning of an AR gene. Isolation of an AR gene involves the isolation of those DNA sequences which encode a protein exhibiting AR-associated structures, properties, or activities, for example, an ankyrin-repeat motif and the ability to induce gene expression of PR proteins that limit pathogen infection. Based on the AR genes and polypeptides described herein, the isolation of additional plant AR coding sequences is made possible using standard strategies and techniques that are well known in the art.

In one particular example, the AR sequences described herein may be used, together with conventional screening methods of nucleic acid hybridization screening. Such hybridization techniques and screening procedures are well known to those skilled in the art and are described, for example, in Benton and Davis, Science 196:180, 1977; Grunstein and Hogness, Proc. Natl. Acad. Sci., USA 72:3961, 1975; Ausubel et al. (supra); Berger and Kimmel (supra); and Sambrook et al., Molecular Cloning: A Laboratory Manual, Cold Spring Harbor Laboratory Press, N.Y. In one particular example, all or part of the NPR1 cDNA (described herein) may be used as a probe to screen a recombinant plant DNA library for genes having sequence identity to the AR gene. Hybridizing sequences are detected by plaque or colony hybridization according to the methods described below.

Alternatively, using all or a portion of the amino acid sequence of the AR polypeptide, one may readily design AR-specific oligonucleotide probes, including AR degenerate oligonucleotide probes (i.e., a mixture of all possible coding sequences for a given amino acid sequence). These oligonucleotides may be based upon the sequence of either DNA strand and any appropriate portion of the AR sequence (FIGS. 4 and 5, 7A, and 7B SEQ ID NOS: 1, 2, 3, 13, and 14, respectively). General methods for designing and preparing such probes are provided, for example, in Ausubel et al., 1996, Current Protocols in Molecular Biology, Wiley Interscience, New York, and Berger and Kimmel, Guide to Molecular Cloning Techniques, 1987, Academic Press, New York. These oligonucleotides are useful for AR gene isolation, either through their use as probes capable of hybridizing to AR complementary sequences or as primers for various amplification techniques, for example, polymerase chain reaction (PCR) cloning strategies. If desired, a combination of different oligonucleotide probes may be used for the screening of a recombinant DNA library. The oligonucleotides may be detectably-labeled using methods known in the art and used to probe filter replicas from a recombinant DNA library. Recombinant DNA libraries are prepared according to methods well known in the art, for example, as described in Ausubel et al. (supra), or they may be obtained from commercial sources.

In one particular example of this approach, related AR sequences having greater than 80% identity are detected or isolated using high stringency conditions. High stringency conditions may include hybridization at about 42° C. and about 50% formamide, 0.1 mg/mL sheared salmon sperm DNA, 1% SDS, 2X SSC, 10% Dextran sulfate, a first wash at about 65° C., about 2X SSC, and 1% SDS, followed by a second wash at about 65° C. and about 0.1X SSC. Alternatively, high stringency conditions may include hybridization at about 42° C. and about 50% formamide, 0.1 mg/mL sheared salmon sperm DNA, 0.5% SDS, 5X SSPE, 1X Denhardt's, followed by two washes at room temperature and 2X SSC, 0.1% SDS, and two washes at between 55-60° C. and 0.2X SSC, 0.1% SDS.

In another approach, low stringency hybridization conditions for detecting AR genes having about 40% or greater sequence identity to the AR genes described herein include, for example, hybridization at about 42° C. and 0.1 mg/mL sheared salmon sperm DNA, 1% SDS, 2X SSC, and 10% Dextran sulfate (in the absence of formamide), and a wash at about 37° C. and 6X SSC, about 1% SDS. Alternatively, the low stringency hybridization may be carried out at about 42° C. and 40% formamide, 0.1 mg/mL sheared salmon sperm DNA, 0.5% SDS, 5X SSPE, 1X Denhardt's, followed by two washes at room temperature and 2X SSC, 0.1% SDS and two washes at room temperature and 0.5X SSC, 0.1% SDS. These stringency conditions are exemplary; other appropriate conditions may be determined by those skilled in the art.

If desired, RNA gel blot analysis of total or poly(A+) RNAs isolated from any plant (e.g., those crop plants described herein) may be used to determine the presence or absence of an AR transcript using conventional methods. As an example, a Northern blot of potato RNA was prepared according to standard methods and probed with a 1.96-kb NPR1 HindIII fragment in a hybridization solution containing 50% formamide, 5X SSC, 2.5X Denhardt's solution, and 300 μg/mL salmon sperm DNA at 37° C. Following overnight hybridization, the blot was washed two times for ten minutes each in a solution containing 1X SSC, 0.2% SDS at 37° C. An autoradiogram of the blot demonstrated the presence an NPR1-hybridizing RNA in the potato RNA sample, indicating that this solanaceous crop plant encoded an acquired resistance gene. These results further indicate that AR genes are not restricted to the crucifer Arabidopsis. Isolation of this hybridizing transcript is performed using standard cDNA cloning techniques.

As discussed above, AR oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, AR sequences may be isolated using the PCR “RACE” technique, or Rapid Amplification of cDNA Ends (see, e.g., Innis et al. (supra)). By this method, oligonucleotide primers based on an AR sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., Proc. Natl. Acad. Sci. USA 85:8998, 1988. Exemplary oligonucleotide primers useful for amplifying AR gene sequences include, without limitation:

A. AA(A/G)GA(A/G)GA(T/C)CA(T/C)ACNAA (SEQ ID NO:24);

B. TA(T/C)TG(T/C)AA(T/C)GTNAA(A/G)AC (SEQ ID NO:25);

C. GCCATNGTNGC(T/C)TG(T/C)TT (SEQ ID NO:26);

D. AA(A/G)GTNAA(A/G)AA(A/G)CA(C/T)GT (SEQ ID NO:27);

E. (A/G)AA(C/T)TC(A/G)CANGTNCC(C/T)TTCAT (SEQ ID NO:28).

For each of the above sequences, N is A, T, G or C.

Alternatively, any plant cDNA or cDNA expression library may be screened by functional complementation of an npr mutant (for example, the npr1 mutant described herein) according to standard methods described herein.

Confirmation of a sequence's relatedness to the AR polypeptide family may be accomplished by a variety of conventional methods including, but not limited to, functional complementation assays and sequence comparison of the gene and its expressed product. In addition, the activity of the gene product may be evaluated according to any of the techniques described herein, for example, the functional or immunological properties of its encoded product.

Once an AR sequence is identified, it is cloned according to standard methods and used for the construction of plant expression vectors as described below.

AR Polypeptide Expression

AR polypeptides may be expressed and produced by transformation of a suitable host cell with all or part of an AR cDNA (for example, the cDNA described above) in a suitable expression vehicle or with a plasmid construct engineered for increasing the expression of an AR polypeptide (supra) in vivo.

Those skilled in the field of molecular biology will understand that any of a wide variety of expression systems may be used to provide the recombinant protein. The precise host cell used is not critical to the invention. The AR protein may be produced in a prokaryotic host, for example, E. coli, or in a eukaryotic host, for example, Saccharomyces cerevisiae, mammalian cells (for example, COS 1 or NIH 3T3 cells), or any of a number of plant cells or whole plant including, without limitation, algae, tree species, ornamental species, temperate fruit species, tropical fruit species, vegetable species, legume species, crucifer species, monocots, dicots, or in any plant of commercial or agricultural significance. Particular examples of suitable plant hosts include, but are not limited to, conifers, petunia, tomato, potato, pepper, tobacco, Arabidopsis, lettuce, sunflower, oilseed rape, flax, cotton, sugarbeet, celery, soybean, alfalfa, Medicago, lotus, Vigna, cucumber, carrot, eggplant, cauliflower, horseradish, morning glory, poplar, walnut, apple, grape, asparagus, cassava, rice, maize, millet, onion, barley, orchard grass, oat, rye, and wheat.

Such cells are available from a wide range of sources including the American Type Culture Collection (Rockland, Md.); or from any of a number seed companies, for example, W. Atlee Burpee Seed Co. (Warminster, Pa.), Park Seed Co. (Greenwood, S.C.), Johnny Seed Co. (Albion, Me.), or Northrup King Seeds (Harstville, S.C.). Descriptions and sources of useful host cells are also found in Vasil I. K., Cell Culture and Somatic Cell Genetics of Plants, Vol I, II, III Laboratory Procedures and Their Applications Academic Press, New York, 1984; Dixon, R. A., Plant Cell Culture-A Practical Approach, IRL Press, Oxford University, 1985; Green et al., Plant Tissue and Cell Culture, Academic Press, New York, 1987; and Gasser and Fraley, Science 244:1293, 1989.

For prokaryotic expression, DNA encoding an AR polypeptide is carried on a vector operably linked to control signals capable of effecting expression in the prokaryotic host. If desired, the coding sequence may contain, at its 5′ end, a sequence encoding any of the known signal sequences capable of effecting secretion of the expressed protein into the periplasmic space of the host cell, thereby facilitating recovery of the protein and subsequent purification. Prokaryotes most frequently used are various strains of E. coli; however, other microbial strains may also be used. Plasmid vectors are used which contain replication origins, selectable markers, and control sequences derived from a species compatible with the microbial host. Examples of such vectors are found in Pouwels et al. (supra) or Ausubel et al. (supra). Commonly used prokaryotic control sequences (also referred to as “regulatory elements”) are defined herein to include promoters for transcription initiation, optionally with an operator, along with ribosome binding site sequences. Promoters commonly used to direct protein expression include the beta-lactamase (penicillinase), the lactose (lac) (Chang et al., Nature 198:1056, 1977), the tryptophan (Trp) (Goeddel et al., Nucl. Acids Res. 8:4057, 1980), and the tac promoter systems, as well as the lambda-derived P_{L} promoter and N-gene ribosome binding site (Simatake et al., Nature 292:128, 1981).

One particular bacterial expression system for AR polypeptide production is the E. coli pET expression system (Novagen, Inc., Madison, Wis.). According to this expression system, DNA encoding an AR polypeptide is inserted into a pET vector in an orientation designed to allow expression. Since the AR gene is under the control of the T7 regulatory signals, expression of AR is induced by inducing the expression of T7 RNA polymerase in the host cell. This is typically achieved using host strains which express T7 RNA polymerase in response to IPTG induction. Once produced, recombinant AR polypeptide is then isolated according to standard methods known in the art, for example, those described herein.

Another bacterial expression system for AR polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

For eukaryotic expression, the method of transformation or transfection and the choice of vehicle for expression of the AR polypeptide will depend on the host system selected. Transformation and transfection methods are described, e.g., in Ausubel et al. (supra); Weissbach and Weissbach, Methods for Plant Molecular Biology, Academic Press, 1989; Gelvin et al., Plant Molecular Biology Manual, Kluwer Academic Publishers, 1990; Kindle, K., Proc. Natl. Acad. Sci., U.S.A. 87:1228, 1990; Potrykus, I., Annu. Rev. Plant Physiol. Plant Mol. Biology 42:205, 1991; and BioRad (Hercules, Calif.) Technical Bulletin #1687 (Biolistic Particle Delivery Systems). Expression vehicles may be chosen from those provided, e.g., in Cloning Vectors: A Laboratory Manual (P. H. Pouwels et al., 1985, Supp. 1987); Gasser and Fraley (supra); Clontech Molecular Biology Catalog (Catalog 1992/93 Tools for the Molecular Biologist, Palo Alto, Calif.); and the references cited above. Other expression constructs are described by Fraley et al. (U.S. Pat. No. 5,352,605).

Construction of Plant Transgenes

Most preferably, an AR polypeptide is produced by a stably-transfected plant cell line, a transiently-transfected plant cell line, or by a transgenic plant. A number of vectors suitable for stable or extrachromosomal transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et al. (supra).

Typically, plant expression vectors include (1) a cloned plant gene under the transcriptional control of 5′ and 3′ regulatory sequences and (2) a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Once the desired AR nucleic acid sequence is obtained as described above, it may be manipulated in a variety of ways known in the art. For example, where the sequence involves non-coding flanking regions, the flanking regions may be subjected to mutagenesis.

The AR DNA sequence of the invention may, if desired, be combined with other DNA sequences in a variety of ways. The AR DNA sequence of the invention may be employed with all or part of the gene sequences normally associated with the AR protein. In its component parts, a DNA sequence encoding an AR protein is combined in a DNA construct having a transcription initiation control region capable of promoting transcription and translation in a host cell.

In general, the constructs will involve regulatory regions functional in plants which provide for modified production of AR protein as discussed herein. The open reading frame coding for the AR protein or functional fragment thereof will be joined at its 5′ end to a transcription initiation regulatory region such as the sequence naturally found in the 5′ upstream region of the AR structural gene. Numerous other transcription initiation regions are available which provide for constitutive or inducible regulation.

For applications where developmental, cell, tissue, hormonal, or environmental expression is desired, appropriate 5′ upstream non-coding regions are obtained from other genes, for example, from genes regulated during meristem development, seed development, embryo development, or leaf development.

Regulatory transcript termination regions may also be provided in DNA constructs of this invention as well. Transcript termination regions may be provided by the DNA sequence encoding the AR protein or any convenient transcription termination region derived from a different gene source. The transcript termination region will contain preferably at least 1-3 kb of sequence 3′ to the structural gene from which the termination region is derived. Plant expression constructs having AR as the DNA sequence of interest for expression (in either the sense or antisense orientation) may be employed with a wide variety of plant life, particularly plant life involved in the production of storage reserves (for example, those involving carbon and nitrogen metabolism). Such genetically-engineered plants are useful for a variety of industrial and agricultural applications as discussed infra. Importantly, this invention is applicable to dicotyledons and monocotyledons, and will be readily applicable to any new or improved transformation or regeneration method.

The expression constructs include at least one promoter operably linked to at least one AR gene. An example of a useful plant promoter according to the invention is a caulimovirus promoter, for example, a cauliflower mosaic virus (CaMV) promoter. These promoters confer high levels of expression in most plant tissues, and the activity of these promoters is not dependent on virally encoded proteins. CaMV is a source for both the 35S and 19S promoters. Examples of plant expression constructs using these promoters are found in Fraley et al., U.S. Pat. No. 5,352,605. In most tissues of transgenic plants, the CaMV 35S promoter is a strong promoter (see, e.g., Odell et al., Nature 313:810, 1985). The CaMV promoter is also highly active in monocots (see, e.g., Dekeyser et al., Plant Cell 2:591, 1990; Terada and Shimamoto, Mol. Gen. Genet. 220:389, 1990). Moreover, activity of this promoter can be further increased (i.e., between 2-10 fold) by duplication of the CaMV 35S promoter (see e.g., Kay et al., Science 236:1299, 1987; Ow et al., Proc. Natl. Acad. Sci., U.S.A. 84:4870, 1987; and Fang et al., Plant Cell 1:141, 1989, and McPherson and Kay, U.S. Pat. No. 5,378,142).

Other useful plant promoters include, without limitation, the nopaline synthase (NOS) promoter (An et al., Plant Physiol. 88:547, 1988 and Rodgers and Fraley, U.S. Pat. No. 5,034,322), the octopine synthase promoter (Fromm et al., Plant Cell 1:977, 1989), figwort mosiac virus (FMV) promoter (Rodgers, U.S. Pat. No. 5,378,619), and the rice actin promoter (Wu and McElroy, W091/09948).

Exemplary monocot promoters include, without limitation, commelina yellow mottle virus promoter, sugar cane badna virus promoter, rice tungro bacilliform virus promoter, maize streak virus element, and wheat dwarf virus promoter.

For certain applications, it may be desirable to produce the AR gene product in an appropriate tissue, at an appropriate level, or at an appropriate developmental time. For this purpose, there are an assortment of gene promoters, each with its own distinct characteristics embodied in its regulatory sequences, shown to be regulated in response to inducible signals such as the environment, hormones, and/or developmental cues. These include, without limitation, gene promoters that are responsible for heat-regulated gene expression (see, e.g., Callis et al., Plant Physiol. 88:965, 1988; Takahashi and Komeda, Mol. Gen. Genet. 219:365, 1989; and Takahashi et al. Plant J. 2:751, 1992), light-regulated gene expression (e.g., the pea rbcS-3A described by Kuhlemeier et al., Plant Cell 1:471, 1989; the maize rbcS promoter described by Schäffner and Sheen, Plant Cell 3:997, 1991; the chlorophyll a/b-binding protein gene found in pea described by Simpson et al., EMBO J. 4:2723, 1985; the Arabssu promoter; or the rice rbs promoter), hormone-regulated gene expression (for example, the abscisic acid (ABA) responsive sequences from the Em gene of wheat described by Marcotte et al., Plant Cell 1:969, 1989; the ABA-inducible HVA1 and HVA22, and rd29A promoters described for barley and Arabidopsis by Straub et al., Plant Cell 6:617, 1994 and Shen et al., Plant Cell 7:295, 1995; and wound-induced gene expression (for example, of wunI described by Siebertz et al., Plant Cell 1:961, 1989), organ-specific gene expression (for example, of the tuber-specific storage protein gene described by Roshal et al., EMBO J. 6:1155, 1987; the 23-kDa zein gene from maize described by Schernthaner et al., EMBO J. 7:1249, 1988; or the French bean β-phaseolin gene described by Bustos et al., Plant Cell 1:839, 1989), or pathogen-inducible promoters (for example, PR-1, prp-1, or β-1,3 glucanase promoters, the fungal-inducible wirla promoter of wheat, and the nematode-inducible promoters, TobRB7-5A and Hmg-1, of tobacco and parsley, respectively).

Plant expression vectors may also optionally include RNA processing signals, e.g, introns, which have been shown to be important for efficient RNA synthesis and accumulation (Callis et al., Genes and Dev. 1:1183, 1987). The location of the RNA splice sequences can dramatically influence the level of transgene expression in plants. In view of this fact, an intron may be positioned upstream or downstream of an AR polypeptide-encoding sequence in the transgene to modulate levels of gene expression.

In addition to the aforementioned 5′ regulatory control sequences, the expression vectors may also include regulatory control regions which are generally present in the 3′ regions of plant genes (Thornburg et al., Proc. Natl. Acad. Sci. U.S.A. 84:744, 1987; An et al., Plant Cell 1:115, 1989). For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include genes encoding antibiotic resistance genes, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta® (Hoechst AG, Frankfurt, Germany).

Efficient use of selectable markers is facilitated by a determination of the susceptibility of a plant cell to a particular selectable agent and a determination of the concentration of this agent which effectively kills most, if not all, of the transformed cells. Some useful concentrations of antibiotics for tobacco transformation include, e.g., 75-100 μg/mL (kanamycin), 20-50 μg/mL (hygromycin), or 5-10 μg/mL (bleomycin). A useful strategy for selection of transformants for herbicide resistance is described, e.g., by Vasil et al., supra.

In addition, if desired, the plant expression construct may contain a modified or fully-synthetic structural AR coding sequence which has been changed to enhance the performance of the gene in plants. Methods for constructing such a modified or synthetic gene are described in Fischoff and Perlak, U.S. Pat. No. 5,500,365.

It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rhizogenes) (see, e.g., Lichtenstein and Fuller In: Genetic Engineering, vol 6, PWJ Rigby, ed, London, Academic Press, 1987; and Lichtenstein, C. P., and Draper, J,. In: DNA Cloning, Vol II, D. M. Glover, ed, Oxford, IRI Press, 1985)), (2) the particle delivery system (see, e.g., Gordon-Kamm et al., Plant Cell 2:603 (1990); or BioRad Technical Bulletin 1687, supra), (3) microinjection protocols (see, e.g., Green et al., supra), (4) polyethylene glycol (PEG) procedures (see, e.g., Draper et al., Plant Cell Physiol. 23:451, 1982; or e.g., Zhang and Wu, Theor. Appl. Genet. 76:835, 1988), (5) liposome-mediated DNA uptake (see, e.g., Freeman et al., Plant Cell Physiol. 25:1353, 1984), (6) electroporation protocols (see, e.g., Gelvin et al., supra; Dekeyser et al., supra; Fromm et al., Nature 319:791, 1986; Sheen Plant Cell 2:1027, 1990; or Jang and Sheen Plant Cell 6:1665, 1994), and (7) the vortexing method (see, e.g., Kindle supra). The method of transformation is not critical to the invention. Any method which provides for efficient transformation may be employed. As newer methods are available to transform crops or other host cells, they may be directly applied. Suitable plants for use in the practice of the invention include, but are not limited to, sugar cane, wheat, rice, maize, sugar beet, potato, barley, manioc, sweet potato, soybean, sorghum, cassava, banana, grape, oats, tomato, millet, coconut, orange, rye, cabbage, apple, watermelon, canola, cotton, carrot, garlic, onion, pepper, strawberry, yam, peanut, onion, bean, pea, mango, citrus plants, walnuts, and sunflower.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast.

In general, transfer and expression of transgenes in plant cells are now routine practices to those skilled in the art, and have become major tools to carry out gene expression studies in plants and to produce improved plant varieties of agricultural or commercial interest.

Transgenic Plant Regeneration

Plant cells transformed with a plant expression vector can be regenerated, for example, from single cells, callus tissue, or leaf discs according to standard plant tissue culture techniques. It is well known in the art that various cells, tissues, and organs from almost any plant can be successfully cultured to regenerate an entire plant; such techniques are described, e.g., in Vasil supra; Green et al., supra; Weissbach and Weissbach, supra; and Gelvin et al., supra.

In one particular example, a cloned AR polypeptide construct under the control of the 35S CaMV promoter and the nopaline synthase terminator and carrying a selectable marker (for example, kanamycin resistance) is transformed into Agrobacterium. Transformation of leaf discs (for example, of tobacco or potato leaf discs), with vector-containing Agrobacterium is carried out as described by Horsch et al. (Science 227:1229, 1985). Putative transformants are selected after a few weeks (for example, 3 to 5 weeks) on plant tissue culture media containing kanamycin (e.g. 100 μg/mL). Kanamycin-resistant shoots are then placed on plant tissue culture media without hormones for root initiation. Kanamycin-resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media. Analysis for the integration of the transgene is accomplished by standard techniques (see, for example, Ausubel et al. supra; Gelvin et al. supra).

Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

Transgenic lines are evaluated for levels of transgene expression. Expression at the RNA level is determined initially to identify and quantitate expression-positive plants. Standard techniques for RNA analysis are employed and include PCR amplification assays using oligonucleotide primers designed to amplify only transgene RNA templates and solution hybridization assays using transgene-specific probes (see, e.g., Ausubel et al., supra). The RNA-positive plants are then analyzed for protein expression by Western immunoblot analysis using AR specific antibodies (see, e.g., Ausubel et al., supra). In addition, in situ hybridization and immunocytochemistry according to standard protocols can be done using transgene-specific nucleotide probes and antibodies, respectively, to localize sites of expression within transgenic tissue.

Ectopic expression of AR genes is useful for the production of transgenic plants having an increased level of resistance to disease-causing pathogens.

In addition, if desired, once the recombinant AR protein is expressed in any cell or in a transgenic plant (for example, as described above), it may be isolated, e.g., using affinity chromatography. In one example, an anti-AR polypeptide antibody (e.g., produced as described in Ausubel et al., supra, or by any standard technique) may be attached to a column and used to isolate the polypeptide. Lysis and fractionation of AR-producing cells prior to affinity chromatography may be performed by standard methods (see, e.g., Ausubel et al., supra). Once isolated, the recombinant protein can, if desired, be further purified, for example, by high performance liquid chromatography (see, e.g., Fisher, Laboratory Techniques In Biochemistry And Molecular Biology, eds., Work and Burdon, Elsevier, 1980).

Polypeptides of the invention, particularly short AR protein fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful AR fragments or analogs.

Ectopic Expression of AR Genes for Engineering Plant Defense Responses to Pathogens

As discussed above, plasmid constructs designed for the expression of AR gene products are useful, for example, for activating plant defense pathways that confer anti-pathogenic properties to a transgenic plant. AR genes that are isolated from a host plant (e.g., Arabidopsis or Nicotiana) may be engineered for expression in the same plant, a closely related species, or a distantly related plant species. For example, the cruciferous Arabidopsis NPR1 gene may be engineered for constitutive low level expression and then transformed into an Arabidopsis host plant. Alternatively, the Arabidopsis NPR1 gene may be engineered for expression in other cruciferous plants, such as the Brassicas (for example, broccoli, cabbage, and cauliflower). Similarly, the NPR1 homolog of Nicotiana glutinosa is useful for expression in related solanaceous plants, such as tomato, potato, and pepper. To achieve pathogen resistance, it is important to express an AR protein at an effective level. Evaluation of the level of pathogen protection conferred to a plant by ectopic expression of an AR gene is determined according to conventional methods and assays.

In one working example, constitutive ectopic expression of the NPR1 gene of Arabidopsis (FIG. 5; SEQ ID NO:2) or the NPR1 homolog of Nicotiana glutinosa (FIG. 7A; SEQ ID NO:13) in Russet Burbank potato is used to control Phytophthora infestans infection. In one particular example, a plant expression vector is constructed that contains an NPR1 cDNA sequence expressed under the control of the enhanced CaMV 35S promoter as described by McPherson and Kay (U.S. Pat. No. 5,359,142). This expression vector is then used to transform Russet Burbank according to the methods described in Fischhoff et al. (U.S. Pat. No. 5,500,365). To assess resistance to fungal infection, transformed Russet Burbank and appropriate controls are grown to approximately eight-weeks-old, and leaves (for example, the second or third from the top of the plant) are inoculated with a mycelial suspension of P. infestans. Plugs of P. infestans mycelia are inoculated on each side of the leaf midvein. Plants are subsequently incubated in a growth chamber at 27° C. with constant fluorescent light.

Leaves of transformed Russet Burbank and control plants are then evaluated for resistance to P. infestans infection according to conventional experimental methods. For this evaluation, the number of lesions per leaf and percentage of leaf area infected are recorded every twenty-four hours for seven days after inoculation. From these data, levels of resistance to P. infestans are determined. Transformed potato plants that express an NPR1 gene having an increased level of resistance to P. infestans relative to control plants are taken as being useful in the invention.

Alternatively, to assess resistance at the whole plant level, transformed and control plants are transplanted to potting soil containing an inoculum of P. infestans. Plants are then evaluated for symptoms of fungal infection (for example, wilting or decayed leaves) over a period of time lasting from several days to weeks. Again, transformed potato plants expressing the NPR1 gene having an increased level of resistance to the fungal pathogen, P. infestans, relative to control plants are taken as being useful in the invention.

In another working example, expression of the NPR1 homolog of Nicotiana glutinosa in tomato is used to control bacterial infection, for example, to Pseudomonas syringae. Specifically, a plant expression vector is constructed that contains the cDNA sequence of the NPR1 homolog from Nicotiana glutinosa (FIG. 7A; SEQ ID NO:13) which is expressed under the control of the enhanced CaMV 35S promoter as described by McPherson and Kay, supra. This expression vector is then used to transform tomato plants according to the methods described in Fischhoff et al., supra. To assess resistance to bacterial infection, transformed tomato plants and appropriate controls are grown, and their leaves are inoculated with a suspension of P. syringae according to standard methods, for example, those described herein. Plants are subsequently incubated in a growth chamber, and the inoculated leaves are subsequently analyzed for signs of disease resistance according to standard methods. For example, the number of chlorotic lesions per leaf and percentage of leaf area infected are recorded and evaluated after inoculation. From a statistical analysis of these data, levels of resistance to P. syringae are determined. Transformed tomato plants that express an NPR1 homolog of Nicotiana glutinosa gene having an increased level of resistance to P. syringae relative to control plants are taken as being useful in the invention.

In still another working example, expression of the NPR1 homolog of rice is used to control fungal diseases, for example, the infection of tissue by Magnaporthe grisea, the cause of rice blast. In one particular approach, a plant expression vector is constructed that contains the cDNA sequence of the rice NPR1 homolog that is constitutively expressed under the control of the rice actin promoter described by Wu et al. (WO 91/09948). This expression vector is then used to transform rice plants according to conventional methods, for example, using the methods described in Hiei et al. (Plant Journal 6:271-282, 1994). To assess resistance to fungal infection, transformed rice plants and appropriate controls are grown, and their leaves are inoculated with a mycelial suspension of M. grisea according to standard methods. Plants are subsequently incubated in a growth chamber and the inoculated leaves are subsequently analyzed for disease resistance according to standard methods. For example, the number of lesions per leaf and percentage of leaf area infected are recorded and evaluated after inoculation. From a statistical analysis of these data, levels of resistance to M. grisea are determined. Transformed rice plants that express a rice NPR1 homolog having an increased level of resistance to M. grisea relative to control plants are taken as being useful in the invention.

AR Interacting Polypeptides

The isolation of AR sequences also facilitates the identification of polypeptides which interact with the AR protein. Such polypeptide-encoding sequences are isolated by any standard two hybrid system (see, for example, Fields et al., Nature 340:245-246, 1989; Yang et al., Science 257:680-682, 1992; Zervos et al., Cell 72:223-232, 1993). For example, all or a part of the AR sequence may be fused to a DNA binding domain (such as the GAL4 or LexA DNA binding domain). After establishing that this fusion protein does not itself activate expression of a reporter gene (for example, a lacZ or LEU2 reporter gene) bearing appropriate DNA binding sites, this fusion protein is used as an interaction target. Candidate interacting proteins fused to an activation domain (for example, an acidic activation domain) are then co-expressed with the AR fusion in host cells, and interacting proteins are identified by their ability to contact the AR sequence and stimulate reporter gene expression. AR-interacting proteins identified using this screening method provide good candidates for proteins that are involved in the acquired resistance signal transduction pathway.

Antibodies

AR polypeptides described herein (or imunogenic fragments or analogs) may be used to raise antibodies useful in the invention; such polypeptides may be produced by recombinant or peptide synthetic techniques (see, e.g., Solid Phase Peptide Synthesis, 2nd ed., 1984, Pierce Chemical Co., Rockford, Ill.; Ausubel et al., supra). The peptides may be coupled to a carrier protein, such as KLH as described in Ausubel et al, supra. The KLH-peptide is mixed with Freund's adjuvant and injected into guinea pigs, rats, or preferably rabbits. Antibodies may be purified by peptide antigen affinity chromatography.

Monoclonal antibodies may be prepared using the AR polypeptides described above and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981; Ausubel et al., supra).

Once produced, polyclonal or monoclonal antibodies are tested for specific AR recognition by Western blot or immunoprecipitation analysis (by the methods described in Ausubel et al., supra). Antibodies which specifically recognize AR polypeptides are considered to be useful in the invention; such antibodies may be used, e.g., in an immunoassay to monitor the level of AR polypeptide produced by a plant.

Use

The invention described herein is useful for a variety of agricultural and commercial purposes including, but not limited to, improving acquired resistance against plant pathogens, increasing crop yields, improving crop and ornamental quality, and reducing agricultural production costs. In particular, ectopic expression of an AR gene in a plant cell provides acquired resistance to plant pathogens and can be used to protect plants from pathogen infestation that reduces plant productivity and viability.

The invention also provides for broad-spectrum pathogen resistance by facilitating the natural mechanism of host resistance. For example, AR transgenes can be expressed in plant cells at sufficiently high levels to initiate an acquired resistance plant defense response constitutively in the absence of signals from the pathogen. The level of expression associated with such a plant defense response may be determined by measuring the levels of defense response gene expression as described herein or according to any conventional method. If desired, the AR transgenes are expressed by a controllable promoter such as a tissue-specific promoter, cell-type specific promoter, or by a promoter that is induced by an external signal or agent such as a pathogen- or wound-inducible control element, thus limiting the temporal or tissue expression or both of an acquired resistance defense response. The AR genes may also be expressed in roots, leaves, or fruits, or at a site of a plant that is susceptible to pathogen penetration and infection.

The invention is also useful for controlling plant disease by enhancing a plant's SAR defense mechanisms. In particular, the invention is useful for combating diseases known to be inhibited by plant SAR defense mechanisms. These include, without limitation, viral diseases caused by TMV and TNV, bacterial diseases caused by Pseudomonas and Xanthomonas, and fungal diseases caused by Erysiphe, Peronospora, Phytophthora, Colletotrichum, and Magnaporthe grisea. In particular exemplary approaches, constitutive or inducible expression of an AR gene in a transgenic plant is useful for controlling powdery mildew of wheat caused by Erysiphe, bacterial leaf spot of pepper caused by Xanthomonas campestris, bacterial wilt and bacterial spot of tomato caused by Pseudomonas syringae and Xanthomonas campestris, and bacterial blights of citrus and walnut caused by Xanthomonas campestris.

### Other Embodiments

The invention further includes analogs of any naturally-occurring plant AR polypeptide. Analogs can differ from the naturally-occurring AR protein by amino acid sequence differences, by post-translational modifications, or by both. Analogs of the invention will generally exhibit at least 40%, more preferably 50%, and most preferably 60% or even having 70%, 80%, or 90% identity with all or part of a naturally-occurring plant AR amino acid sequence. The length of sequence comparison is at least 15 amino acid residues, preferably at least 25 amino acid residues, and more preferably more than 35 amino acid residues. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring AR polypeptide by alterations in primary sequence. These include genetic variants, both natural and induced (for example, resulting from random mutagenesis by irradiation or exposure to ethyl methylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the invention also includes AR polypeptide fragments. As used herein, the term “fragment,” means at least 20 contiguous amino acids, preferably at least 30 contiguous amino acids, more preferably at least 50 contiguous amino acids, and most preferably at least 60 to 80 or more contiguous amino acids. Fragments of AR polypeptides can be generated by methods known to those skilled in the art or may result from normal protein processing (e.g., removal of amino acids from the nascent polypeptide that are not required for biological activity or removal of amino acids by alternative mRNA splicing or alternative protein processing events). In preferred embodiments, an AR polypeptide fragment includes an ankyrin-repeat motif as described herein. In other preferred embodiments, an AR fragment is capable of interacting with a second polypeptide component of the AR signal transduction cascade.

Furthermore, the invention includes nucleotide sequences that facilitate specific detection of an AR nucleic acid. Thus, AR sequences described herein or portions thereof may be used as probes to hybridize to nucleotide sequences from other plants (e.g., dicots, monocots, gymnosperms, and algae) by standard hybridization techniques under conventional conditions. Sequences that hybridize to an AR coding sequence or its complement and that encode an AR polypeptide are considered useful in the invention. As used herein, the term “fragment,” as applied to nucleic acid sequences, means at least 5 contiguous nucleotides, preferably at least 10 contiguous nucleotides, more preferably at least 20 to 30 contiguous nucleotides, and most preferably at least 40 to 80 or more contiguous nucleotides. Fragments of AR nucleic acid sequences can be generated by methods known to those skilled in the art.

Deposit

Cosmids 21A4-2-1, 21A4-4-3-1, 21A4-P5-1 have been deposited with the American Type Culture Collection on Jul. 8, 1996, and bear the accession numbers ATCC No. 97649, 97650, and 97651. Plasmid pKExNPR1 was deposited on Jul. 31, 1996 and bears the accession number ATCC No. 97671. Applicants acknowledge their responsibility to replace these plasmids should it loose viability before the end of the term of a patent issued hereon, and their responsibility to notify the American Type Culture Collection of the issuance of such a patent, at which time the deposit will be made available to the public. Prior to that time the deposit will be made available to the Commissioner of Patents under terms of 37 CFR § 1.14 and 35 USC § 112. These deposits are available as required by foreign patent laws in countries wherein counterparts of this subject application, or progeny, are filed. It should be understood that availability of a deposit does not constitute a license to practice the subject invention.

All publications and patent applications mentioned in this specification are herein incorporated by reference to the same extent as if each independent publication or patent application was specifically and individually indicated to be incorporated by reference.

We claim:

1. An isolated nucleic acid molecule including a sequence encoding an acquired resistance polypeptide, wherein said acquired resistance polypeptide is capable of conferring, on a plant expressing said polypeptide, resistance to a plant pathogen.

2. The isolated nucleic acid molecule of claim 1, wherein said polypeptide is capable of mediating the expression of a pathogenesis-related polypeptide.

3. The isolated nucleic acid molecule of claim 1, wherein said polypeptide comprises an ankyrin-repeat motif.

4. The isolated nucleic acid molecule of claim 1, wherein said polypeptide is obtained from an angiosperm.

5. The isolated nucleic acid molecule of claim 4, wherein said angiosperm is a member of the Solanaceae.

6. The isolated nucleic acid molecule of claim 4, wherein said angiosperm is a member of the Cruciferae.

7. The isolated nucleic acid molecule of claim 1, wherein said nucleic acid molecule is genomic DNA.

8. The isolated nucleic acid molecule of claim 1, wherein said nucleic acid molecule is cDNA.

9. The isolated nucleic acid molecule of claim 1, wherein said plant pathogen is a bacterium, virus, viroid, fungus, nematode, or insect.

10. An isolated nucleic acid molecule that encodes an acquired resistance polypeptide that specifically hybridizes to a nucleic acid molecule comprising the genomic nucleic acid sequence of FIG. 4 (SEQ ID NO:1).

11. An isolated nucleic acid molecule that encodes an acquired resistance polypeptide that specifically hybridizes to a nucleic acid molecule comprising the cDNA of FIG. 5 (SEQ ID NO:2).

12. An isolated nucleic acid molecule that encodes an acquired resistance polypeptide that specifically hybridizes to a nucleic acid molecule comprising the DNA sequence of FIG. 7A (SEQ ID NO:13).

13. The isolated nucleic acid molecule of claims 10-12, wherein said nucleic acid molecule encodes a polypeptide that mediates the expression of a pathogenesis-related polypeptide.

14. The isolated nucleic acid molecule of claims 10-12, wherein said nucleic acid molecule encodes a polypeptide comprising an ankyrin-repeat motif.

15. The isolated nucleic acid molecule of claim 1 or 10-12, wherein said nucleic acid molecule is operably linked to an expression control region.

16. A vector comprising the nucleic acid molecule of claim 1 or 10-12, said vector being capable of directing expression of the polypeptide encoded by said nucleic acid molecule.

17. A cell comprising an isolated nucleic acid molecule of claim 1, 10-12, or 16.

18. The cell of claim 17, wherein said cell is a plant cell.

19. The cell of claim 17, wherein said cell is a bacterial cell.

20. The cell of claim 19, wherein said bacterial cell is Agrobacterium.

21. The cell of claim 18, wherein said plant cell has increased resistance to a plant pathogen.

22. A transgenic plant comprising a nucleic acid molecule of claim 1, 10-12, or 16, wherein said nucleic acid molecule is expressed in said transgenic plant.

23. The transgenic plant of claim 22, wherein said transgenic plant is an angiosperm.

24. The transgenic plant of claim 22, wherein said transgenic angiosperm is a dicot.

25. The transgenic plant of claim 24, wherein said dicot is a cruciferous plant.

26. The transgenic plant of claim 24, wherein said dicot is a solanaceous plant.

27. The transgenic plant of claim 23, wherein said transgenic angiosperm is a monocot.

28. A seed from a transgenic plant of claim 22.

29. A cell from a transgenic plant of claim 22.

30. A substantially pure acquired resistance polypeptide including an amino acid sequence that has at least 40% identity to the amino acid sequence of FIG. 5 (SEQ ID NO:3) or FIG. 7B (SEQ ID NO:14).

31. The of substantially pure polypeptide claim 30, wherein said polypeptide is capable of mediating the expression of a pathogenesis-related polypeptide.

32. The substantially pure polypeptide of claim 30, wherein said polypeptide includes an ankyrin-repeat motif or a G-protein coupled receptor motif.

33. The substantially pure polypeptide of claim 30, wherein said polypeptide is obtained from an angiosperm.

34. The substantially pure polypeptide of claim 33, wherein said angiosperm is a member of the Solanaceae.

35. The substantially pure polypeptide of claim 33, wherein said angiosperm is a member of the Cruciferae.

36. A method of producing an acquired resistance polypeptide, said method comprising the steps of:
(a) providing a cell transformed with a nucleic acid molecule of claim 1; 10-12, or 16 positioned for expression in the cell;
(b) culturing the transformed cell under conditions for expressing the nucleic acid molecule; and
(c) recovering the acquired resistance polypeptide.

37. A recombinant acquired resistance polypeptide produced by the method of claim 31.

38. A substantially pure antibody that specifically recognizes and binds to an acquired resistance polypeptide or a portion thereof.

39. The substantially pure antibody of claim 38, wherein said antibody recognizes and binds to a recombinant acquired resistance polypeptide or a portion thereof.

40. A method of providing an increased level of resistance against a disease caused by a plant pathogen in a transgenic plant, said method comprising the steps of:
(a) producing a transgenic plant cell including the nucleic acid molecule of claim 1, 10-12, or 16 wherein said nucleic acid is positioned for expression in the plant cell; and
(b) growing a transgenic plant from the plant cell wherein the nucleic acid molecule is expressed in the transgenic plant and the transgenic plant is thereby provided with an increased level of resistance against a disease caused by a plant pathogen.

41. The method of claim 40, wherein said plant pathogen is a bacterium, virus, viroid, fungus, nematode, or insect.

42. The method of claim 40, wherein said plant pathogen is Phytophthora, Peronospora, or Pseudomonas.

43. A method of isolating an acquired resistance gene or fragment thereof, said method comprising the steps of:
(a) contacting the nucleic acid molecule of FIG. 4 (SEQ ID NO:1), FIG. 5 (SEQ ID NO:2), or FIG. 7A (SEQ ID NO:13) or a portion thereof with a preparation of DNA from a plant cell under hybridization conditions providing detection of DNA sequences having at least 40% or greater sequence identity to the nucleic acid sequence of FIG. 4 (SEQ ID NO:1), FIG. 5 (SEQ ID NO:2), or FIG. 7A (SEQ ID NO:13); and
(b) isolating said hybridizing DNA.

44. A method of isolating an acquired resistance gene or fragment thereof, said method comprising the steps of:
(a) providing a sample of plant cell DNA;
(b) providing a pair of oligonucleotides having sequence identity to a region of the nucleic acid of FIG. 4 (SEQ ID NO:1), FIG. 5 (SEQ ID NO:2), or FIG. 7A (SEQ ID NO:13);
(c) contacting the pair of oligonucleotides with said plant cell DNA under conditions suitable for polymerase chain reaction-mediated DNA amplification; and
(d) isolating the amplified acquired resistance gene or fragment thereof.

45. The method of claim 44, wherein said amplification step is carried out using a sample of cDNA prepared from a plant cell.

46. The method of claim 44, wherein said pair of oligonucleotides are based on a sequence encoding an acquired resistance polypeptide, wherein the acquired resistance polypeptide is at least 40% identical to the amino acid sequence of FIG. 5 (SEQ ID NO:3) or FIG. 7B (SEQ ID NO:14).


SEQUENCE LISTING (28 sequences)

SEQ ID NO: 1 - DNA (Genomic DNA), 7548 bp; double-stranded, linear
aagcttgtgatgcaagtcatgggatattgctttgtgttaagtatacaaaaccatcacgtggatacatagtcttcaaaccaaccactaaacagtatcaggtcataccaaagccagaagtgaagggttgggatatgtcattgggtttagcggtaatcggattgaaccctttccggtataaaatacaaaggctttcgcagtctcggcgtatgtgtatgtctcggggtatctaccatttgaatcacagaacttttatgtgcgaagttttcgattctgattcgtttacctggaagagattagaaatttgcgtctaccaaaaacagacagattaattttttccaacccgatacaagtttcggggttcttgcattggatatcacggaacaacaatgtgatccggttttgtctcaaaaccgaaacttggtccttcttccatactccgaactctgatgttttctcaggattagtcagatacgaagggaagctaggtgctattcgtcagtggacaaacaaagatcaagaagatgttcacgagttatgggttttaaagagcagttttgaaaagtcgtgggttaaagtgaaagatattaaaagcattggagtagatttgattacgtggactccaagcaacgacgttgtattgtttcgtagtagtgatcgtggttgcctctacaacataaacgcagagaagttgaatttagtttatgcaaaaaaagagggatctgattgttctttcgtttgttttccgttttgttctgattacgagagggttgatctgaacggaagaagcaacgggccgacactttaaaaaaaaaataaaaaaaatgggccgacaaatgcaaacgtagttgacaaggatctcaagtctcaagtctcaattggctcgctcattgtggggcataaatatatctagtgatgtttaattgttttttataaggtaaaaaggaatattgaattttgtttcttaggtttatgtaataataccaaacattgttttatgaatatttaatctgattttttggctagttattttattatatcaagggttcctgtttatagttgaaaacagttactgtatagaaaatagtgtcccaattttctctcttaaataatatattagttaataaaagatattttaatatattagatatacaataatatctaaagcaacacatatttagacacaacacgtaatatcttactattgtttacatatatttatagcttaccaatataacccgtatctatgttttataagcttttatacaatatatgtacggtatgctgtccacgtatatatattctccaaaaaaaacgcatggtacacaaaatttattaaatatttggcaattgggtgtttatctaaagtttatcacaatatttatcaactataatagatggtagaagataaaaaaattatatcagattgattcaattaaattttataatatatcattttaaaaaattaattaaaagaaaactatttcataaaattgttcaaaagataattagtaaaattaattaaatatgtgatgctattgaattatagagagttattgtaaatttacttaaaatcatacaaatcttatcctaatttaacttatcatttaagaaatacaaaagtaaaaaacgcggaaagcaataatttatttaccttattataactcctatataaagtactctgtttattcaacataatcttacgttgttgtattcataggcatctttaacctatcttttcattttctgatctcgatcgttttcgatccaacaaaatgagtctaccggtgaggaaccaagaggtgattatgcagattccttcttcttctcagtttccagcaacatcgagtccggaaaacaccaatcaagtgaaggatgagccaaatttgtttagacgtgttatgaatttgcttttacgtcgtagttattgaaaaagctgatttatcgcatgattcagaacgagaagttgaaggcaaataactaaagaagtcttttatatgtatacaataattgtttttaaatcaaatcctaattaaaaaaatatattcattatgactttcatgtttttaatgtaatttattcctatatctataatgatttttgttgtgaagagcgttttcatttgctatagaacaaggagaatagttccaggaaatattcgacttgatttaattatagtgtaaacatgctgaacactgaaaattactttttcaataaacgaaaaatataatatacattacaaaacttatgtgaataaagcatgagacttaatatacgttccctttatcattttacttcaaagaaaataaacagaaatgtaactttcacatgtaaatctaattcttaaatttaaaaaataatatttatatatttatatgaaaataacgaaccggatgaaaaataaattttatatatttatatcatctccaaatctagtttggttcaggggcttaccgaaccggattgaacttctcatatacaaaaattagcaacacaaaatgtctccggtataaatactaacatttataacccgaaccggtttagcttcctgttatatctttttaaaaaagatctctgacaaagattcctttcctggaaatttaccggttttggtgaaatgtaaaccgtgggacgaggatgcttcttcatatctcaccaccactctcgttgactggacttggctctgctcgtcaatggttatcttcgatcttaaaccaaatccagttgataaggtctcttcgttgattagcagagatctctttaatttgtgaatttcaattcatcggaacctgttgatggacaccaccattgatggattcgccgattcttatgaaatcagcagcactagtttcgtcgctaccgataacaccgactcctctattgtttatctggccgccgaacaagtactcaccggacctgatgtatctgctctgcaattgctctccaacagcttcgaatccgtctttgactcgccggatgatttctacagcgacgctaagcttgttctctccgacggccgggaagtttctttccaccggtgcgttttgtcagcgagaagctctttcttcaagagcgctttagccgccgctaagaaggagaaagactccaacaacaccgccgccgtgaagctcgagcttaaggagattgccaaggattacgaagtcggtttcgattcggttgtgactgttttggcttatgtttacagcagcagagtgagaccgccgcctaaaggagtttctgaatgcgcagacgagaattgctgccacgtggcttgccggccggcggtggatttcatgttggaggttctctatttggctttcatcttcaagatccctgaattaattactctctatcaggtaaaacaccatctgcattaagctatggttacacattcatgaatatgttcttacttgagtacttgtatttgtatttcagaggcacttattggacgttgtagacaaagttgttatagaggacacattggttatactcaagcttgctaatatatgtggtaaagcttgtatgaagctattggatagatgtaaagagattattgtcaagtctaatgtagatatggttagtcttgaaaagtcattgccggaagagcttgttaaagagataattgatagacgtaaagagcttggtttggaggtacctaaagtaaagaaacatgtctcgaatgtacataaggcacttgactcggatgatattgagttagtcaagttgcttttgaaagaggatcacaccaatctagatgatgcgtgtgctcttcatttcgctgttgcatattgcaatgtgaagaccgcaacagatcttttaaaacttgatcttgccgatgtcaaccataggaatccgaggggatatacggtgcttcatgttgctgcgatgcggaaggagccacaattgatactatctctattggaaaaaggtgcaagtgcatcagaagcaactttggaaggtagaaccgcactcatgatcgcaaaacaagccactatggcggttgaatgtaataatatcccggagcaatgcaagcattctctcaaaggccgactatgtgtagaaatactagagcaagaagacaaacgagaacaaattcctagagatgttcctccctcttttgcagtggcggccgatgaattgaagatgacgctgctcgatcttgaaaatagaggtatctatcaagtcttatttcttatatgtttgaattaaatttatgtcctctctattaggaaactgagtgaactaatgataactattctttgtgtcgtccactgtttagttgcacttgctcaacgtctttttccaacggaagcacaagctgcaatggagatcgccgaaatgaagggaacatgtgagttcatagtgactagcctcgagcctgaccgtctcactggtacgaagagaacatcaccgggtgtaaagatagcacctttcagaatcctagaagagcatcaaagtagactaaaagcgctttctaaaaccggtatggattctcacccacttcatcggactccttatcacaaaaaacaaaactaaatgatctttaaacatggttttgttacttgctgtctgaccttgtttttttatcatcagtggaactcgggaaacgattcttcccgcgctgttcggcagtgctcgaccagattatgaactgtgaggacttgactcaactggcttgcggagaagacgacactgctgaagaaacgactacaaaagaagcaaaggtacatggaaatacaagagacactaaagaaggcctttagtgaggacaatttggaattaggaaattcgtccctgacagattcgacttcttccacatcgaaatcaaccggtggaaagaggtctaaccgtaaactctctcatcgtcgtcggtgagactcttgcctcttagtgtaatttttgctgtaccatataattctgttttcatgatgactgtaactgtttatgtctatcgttggcgtcatatagtttcgctcttcgttttgcatcctgtgtattattgctgcaggtgtgcttcaaacaaatgttgtaacaatttgaaccaatggtatacagatttgtaatatatatttatgtacatcaacaataacccatgatggtgttacagagttgctagaatcaaagtgtgaaataatgtcaaattgttcatctgttggatattttccaccaagaaccaaaagaatattcaagttccctgaacttctggcaacattcatgttatatgtatcttcctaattcttcctttaaccttttgtaactcgaattacacagcaagttagtttcaggtctagagataagagaacactgagtgggcgtgtaaggtgcattctcctagtcagctccattgcatccaacatttgtgaatgacacaagttaacaatcctttgcaccatttctgggtgcatacatggaaacttcttcgattgaaacttcccacatgtgcaggtgcgttcgctgtcactgatagaccaagagactgaaagctttcacaaattgccctcaaatcttctgtttctatcgtcatgactccatatctccgaccactggtcatgagccagagcccactgattttgagggaattgggctaaccatttccgagcttctgagtccttctttttgatgtcctttatgtaggaatcaaattcttccttctgacttgtggatccagcctgcttcacaaggctcaccaggttgtagtctccaaaaatatcatggaattgtaagcaaaaacaatccagacagaacctgtgatagacccaaggttcttgccacagtgatccgggttcgttaataacagcaactatgtccgggtgaggactggagacgaagcaaacgtctttcctttgtgttaccttctctctgatattagtgagaaaccaacgccaactatcagtggacacttctttggtaagcggaaagcaagcgggaaaaacaatcatcagcgtcgagtcctgaggaaaatcatcaatttcataggggtacttgccgttcaagtcttttgaatccactatgatcagaggtctacagtgttgaaacccttcaatggactgtggaaacgcccaaaacgcgccaccgaaggatgcaaattcaggattagggaaaagctcatattgcagtccacaagtagcccattagatgagtgaaatgcagccaattagtttaggcaatactctgaaactctgatctttgattacttcctgttctgctgcccgcagctttgaagttttaagcatgtcaccaaacttttcaactctgctgttagagtgggttgtaccctgatcagacactcaatctcttctgctgcaaattacaagttgaagttttccggcttaatagaacaacaagtatgtggaccaactacacttagttatcttaacaagtccatgttcttctattcaatctgcccgacgcgaccaattgcatttccatctgatgcatttaaacgtatactcgtccttctcaatctcttgtactacacacttttgctgccctctaatggaacaccagtccaccgccttcttcagctcatccctatctttaaaacacaaccctacacgcaattcatgatcatcaatccacaaactagacaaagtacactgttttgaagcactcgaatcaacaacacctttacttaataagcacgcatacggtaatacctctaagcctggcacattcaaaccttgtgtgcatcatctgaacccgagtttttatccgttatttctccatccccacctccacgagtgctaccatttccgaagtcagaattttcctcgtcttcaatccacccgttactgttacccactccctgaacctctaaaccattatctctctctactttcacagatgcatgtgacacataatcagtagcttcttggggttgttgcgtcctctgtgtattcgaggaactagcgggatattctattacggatgaacaagcagcatgatcagtaacattatcagatgtcgatttcacttccaaatacaactccacatttcttatagaaggatgataacttggaacttcaagcatagtctccaaactagtgtcgttcactacatgaagaagtagatagataaagagatccggtgaaacaactacaggatacttaccaaaatatattgaacactgatttctgcagctgcaatccaaaaattggataaagaccattcaacaatgtacttaacgcagtcttttgcctaaccttgaccgttttaggagtggatccttcatagtaaacaccatcaggaccatacttggtagaacctttctctcaaggtttccatcgccatgaccataacagtcctgcagtgaattctaagaaaaatgtaaaaaattttggcctaaactcataattcttaacatacgaaaccatggagaactccatgtctaaaaaataaaggctaaagctttttggcgacagaagcagataaatccattcaaaacacataaactctaaacaataaacagtgatactcaatactaagacttgtaaaggtctacgtaactcaaaactggagaattgtcagatcgggtgtggctagtagaagctt

SEQ ID NO: 2 - DNA (cDNA), 2104 bp; double-stranded, linear
  Coding Sequence 93...1871
tcgatctttaaccaaatccagttgataaggtctcttcgttgattagcagagatctctttaatttgtgaatttcaattcatcggaacctgttgatggacaccaccattgatggattcgccgattcttatgaaatcagcagcactagtttcgtcgctaccgataacaccgactcctctattgtttatctggccgccgaacaagtactcaccggacctgatgtatctgctctgcaattgctctccaacagcttcgaatccgtctttgactcgccggatgatttctacagcgacgctaagcttgttctctccgacggccgggaagtttctttccaccggtgcgttttgtcagcgagaagctctttcttcaagagcgctttagccgccgctaagaaggagaaagactccaacaacaccgccgccgtgaagctcgagcttaaggagattgccaaggattacgaagtcggtttcgattcggttgtgactgttttggcttatgtttacagcagcagagtgagaccgccgcctaaaggagtttctgaatgcgcagacgagaattgctgccacgtggcttgccggccggcggtggatttcatgttggaggttctctatttggctttcatcttcaagatccctgaattaattactctctatcagaggcacttattggacgttgtagacaaagttgttatagaggacacattggttatactcaagcttgctaatatatgtggtaaagcttgtatgaagctattggatagatgtaaagagattattgtcaagtctaatgtagatatggttagtcttgaaaagtcattgccggaagagcttgttaaagagataattgatagacgtaaagagcttggtttggaggtacctaaagtaaagaaacatgtctcgaatgtacataaggcacttgactcggatgatattgagttagtcaagttgcttttgaaagaggatcacaccaatctagatgatgcgtgtgctcttcatttcgctgttgcatattgcaatgtgaagaccgcaacagatcttttaaaacttgatcttgccgatgtcaaccataggaatccgaggggatatacggtgcttcatgttgctgcgatgcggaaggagccacaattgatactatctctattggaaaaaggtgcaagtgcatcagaagcaactttggaaggtagaaccgcactcatgatcgcaaaacaagccactatggcggttgaatgtaataatatcccggagcaatgcaagcattctctcaaaggccgactatgtgtagaaatactagagcaagaagacaaacgagaacaaattcctagagatgttcctccctcttttgcagtggcggccgatgaattgaagatgacgctgctcgatcttgaaaatagagttgcacttgctcaacgtctttttccaacggaagcacaagctgcaatggagatcgccgaaatgaagggaacatgtgagttcatagtgactagcctcgagcctgaccgtctcactggtacgaagagaacatcaccgggtgtaaagatagcacctttcagaatcctagaagagcatcaaagtagactaaaagcgctttctaaaaccgtggaactcgggaaacgattcttcccgcgctgttcggcagtgctcgaccagattatgaactgtgaggacttgactcaactggcttgcggagaagacgacactgctgagaaacgactacaaaagaagcaaaggtacatggaaatacaagagacactaaagaaggcctttagtgaggacaatttggaattaggaaattcgtccctgacagattcgacttcttccacatcgaaatcaaccggtggaaagaggtctaaccgtaaactctctcatcgtcgtcggtgagactcttgcctcttagtgtaatttttgctgtaccatataattctgttttcatgatgactgtaactgtttatgtctatcgttggcgtcatatagtttcgctcttcgttttgcatcctgtgtattattgctgcaggtgtgcttcaaacaaatgttgtaacaatttgaaccaatggtatacagatttgtaatatatatttatgtacatcaacaataaaaaaaaaaaaaaaaaa

SEQ ID NO: 3 - protein, 593 aa
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 4 - protein, 49 aa
NHRNPRGYTVLHVAAMRKEPQLILSLLEKGASASEATLEGRTALMIAKQ

SEQ ID NO: 5 - protein, 49 aa
NAKTKNGYTALHQAAQQGHTHIINVLLQNNASPNELTVNGNTALAIARR

SEQ ID NO: 6 - protein, 28 aa
KVKKHVSNVHKALDSDDIELVKLLLKED

SEQ ID NO: 7 - protein, 28 aa
KTKNGLSPLHMATQGDHLNCVQLLLSRN

SEQ ID NO: 8 - protein, 33 aa
KHVSNVHKALDSDDIELVKLLLKEDHTNLDDAC

SEQ ID NO: 9 - protein, 33 aa
DDACALHFAVAYCNVKTATDLLKLDLADVNHRN

SEQ ID NO: 10 - protein, 33 aa
RGYTVLHVAAMRKEPQLILSLLEKGASASEATL

SEQ ID NO: 11 - protein, 33 aa
EGRTALMIAKQATMAVECNNIPEQCKHSLKGRL

SEQ ID NO: 12 - protein, 55 aa
GTPLHLAARGHVEVVKLLLDGADVNATKAISQNNLDIAEVKNPDDVKTMRQSINE

SEQ ID NO: 13 - DNA (cDNA), 2172 bp; double-stranded, linear
gtgactttctaactatggctgaaattgcagaacgaaaaagactttccatttttcacttgaatgaaacccaaaatggaaatctatctctcttcttcttctcttttactacctccatttccatggctttccctcctctaccttccctagctcttttcaatttctagaatattcttttcttagtctgtaattatctatagctcaatttctaagacagaacttatgtaaggcggctttctgtaatggataatagtaggactgcgttttctgattcgaatgacatcagcggaagcagtagtatatgctgcatcggcggcggcatgactgaatttttctcgccggagacttcgccggcggagatcacttcactgaaacgcctatcggaaacactggaatctatcttcgatgcgtctttgccggagtttgactacttcgccgacgctaagcttgtggtttccggcccgtgtaaggaaattccggtgcaccggtgcattttgtcggcgaggagtccgttctttaagaatttgttctgcggtaaaaaggagaagaatagtagtaaggtggaattgaaggaggtgatgaaagagcatgaggtgagctatgatgctgtaatgagtgtattggcttatttgtatagtggtaaagttaggccttcacctaaagatgtgtgtgtttgtgtggacaatgactgctctcatgtggcttgtaggccagctgtggcattcctggttgaggttttgtacacatcatttacctttcagatctctgaattggttgacaagtttcagagacacctactggatattcttgacaaaactgcagcagacgatgtaatgatggttttatctgttgcaaacatttgtggtaaagcatgcgagagattgctttcaagctgcattgagattattgtcaagtctaatgttgatatcataacccttgataaagccttgcctcatgacattgtaaaacaaattactgattcacgagcggaacttggtctacaagggcctgaaagcaacggttttcctgataaacatgttaagaggatacatagggcattggattctgatgatgttgaattactacaaatgttgctaagagaggggcatactaccctagatgatgcatatgctctccattatgctgtagcgtattgcgatgcaaagactacagcagaacttctagatcttgcacttgctgatattaatcatcaaaattcaaggggatacacggtgctgcatgttgcagccatgaggaaagagcctaaaattgtagtgtcccttttaaccaaaggagctagaccttctgatctgacatccgatggaagaaaagcacttcaaatcgccaagaggctcactaggcttgtggatttcagtaagtctccggaggaaggaaaatctgcttcgaatgatcggttatgcattgagattctggagcaagcagaaagaagagaccctctgctaggagaagcttctgtatctcttgctatggcaggcgatgatttgcgtatgaagctgttataccttgaaaatagagttggcctggctaaactcctttttccaatggaagctaaagttgcaatggacattgctcaagttgatggcacttctgagttcccactggctagcatcggcaaaaagatggctaatgcacagaggacaacagtagatttgaacgaggctcctttcaagataaaagaggagcacttgaatcggcttagagcactctctagaactgtagaacttggaaaacgcttctttccacgttgttcagaagttctaaataagatcatggatgctgatgacttgtctgagatagcttacatggggaatgatacggcagaagagcgtcaactgaagaagcaaaggtacatggaacttcaagaaattctgactaaagcattcactgaggataaagaagaatatgataagactaacaacatctcctcatcttgttcctctacatctaagggagtagataagcccaataagctcccttttaggaaataggtaattgtattaggatatatgaggaagaagaggattttcttgtaacatagcactctttcctttcatcatttgatatgtcaacatacatacaacagctgtaccataaacttgtattgttgcacttacaactttgaagaacagaatttatttgaaaaaaaaaaaaaaa

SEQ ID NO: 14 - protein, 588 aa
MDNSRTAFSDSNDISGSSSICCIGGGMTEFFSPETSPAEITSLKRLSETLESIFDASLPEFDYFADAKLVVSGPCKEIPVHRCILSARSPFFKNLFCGKKEKNSSKVELKEVMKEHEVSYDAVMSVLAYLYSGKVRPSPKDVCVCVDNDCSHVACRPAVAFLVEVLYTSFTFQISELVDKFQRHLLDILDKTAADDVMMVLSVANICGKACERLLSSCIEIIVKSNVDIITLDKALPHDIVKQITDSRAELGLQGPESNGFPDKHVKRIHRALDSDDVELLQMLLREGHTTLDDAYALHYAVAYCDAKTTAELLDLALADINHQNSRGYTVLHVAAMRKEPKIVVSLLTKGARPSDLTSDGRKALQIAKRLTRLVDFSKSPEEGKSASNDRLCIEILEQAERRDPLLGEASVSLAMAGDDLRMKLLYLENRVGLAKLLFPMEAKVAMDIAQVDGTSEFPLASIGKKMANAQRTTVDLNEAPFKIKEEHLNRLRALSRTVELGKRFFPRCSEVLNKIMDADDLSEIAYMGNDTAEERQLKKQRYMELQEILTKAFTEDKEEYDKTNNISSSCSSTSKGVDKPNKLPFRK

SEQ ID NO: 15 - DNA (Genomic DNA), 21 bp; single-stranded, linear
gtgacagacttgctcctactg

SEQ ID NO: 16 - DNA, 20 bp; single-stranded, linear
cagtgtgtatcaaagcacca

SEQ ID NO: 17 - DNA, 21 bp; single-stranded, linear
ttctccagaccacatgattat

SEQ ID NO: 18 - DNA, 21 bp; single-stranded, linear
tgaagctaatatgcacaggag

SEQ ID NO: 19 - DNA, 21 bp; single-stranded, linear
gtaggtgctcttgttcttccc

SEQ ID NO: 20 - DNA, 21 bp; single-stranded, linear
cacataattcccacgaggatc

SEQ ID NO: 21 - protein, 17 aa
MKGTCEFIVTSLEPDRL

SEQ ID NO: 22 - protein, 14 aa
RRKELGLEVPKVKK

SEQ ID NO: 23 - protein, 14 aa
KKQRYMEIQETLKK

SEQ ID NO: 24 - DNA, 17 bp; single-stranded, linear
aargargaycayacnaa

SEQ ID NO: 25 - DNA, 17 bp; single-stranded, linear
taygtyaaygtnaarac

SEQ ID NO: 26 - DNA, 17 bp; single-stranded, linear
gccatngtngcytgytt

SEQ ID NO: 27 - DNA, 17 bp; single-stranded, linear
aargtnaaraarcaygt

SEQ ID NO: 28 - DNA, 21 bp; single-stranded, linear
raaytcrcangtnccyttcat